(19) United States
(12) Patent Application Publication
(10) Pub. No.: US 2024/0002422 A1
(43) Pub. Date: Jan. 4, 2024

(54) Glycan Analysis and Profiling

(71) Applicant: Seagen Inc., Bothell, WA (US)

(72) Inventors: Ana Paula Galvao da Silva, San Diego, CA (US); Mai Zhang, Carlsbad, CA (US); Darius Ghaderi, Laupheim (DE); Julie DeSander, Arlington, MA (US); Jeffrey Behrens, Newton, MA (US); Jillian M. Prendergast, Maynard, MA (US)

(73) Assignee: Seagen Inc., Bothell, WA (US)

(21) Appl. No.: 18/115,264

(22) Filed: Feb. 28, 2023

Related U.S. Application Data
(63) Continuation of application No. 16/850,353, filed on Apr. 16, 2020, now abandoned.
Application No. 16/850,353 is a continuation of application No. 15/518,179, filed on Apr. 10, 2017, now abandoned.
(60) Provisional application No. 62/062,460, filed on Oct. 10, 2014.

(51) Int. Cl. C07H 15/04; G01N 33/574; G01N 33/68; C40B 40/12

(52) U.S. Cl. CPC C07H 15/04; G01N 33/57484; G01N 2400/38; C40B 40/12; G01N 2400/12; G01N 33/6854

(57) ABSTRACT

The invention provides methods and tools, for example, glycan arrays, for the analysis of glycans and anti-glycan antibodies. Embodiments of the invention may be used to detect proteins, antibodies, diseases and/or pathogenic agents. In other embodiments, methods of the invention are used to develop or optimize arrays and antibodies.

### CROSS REFERENCE TO RELATED APPLICATIONS

This application is a continuation of U.S. application Ser. No. 16/850,353 filed Apr. 16, 2020, which is a continuation of U.S. application Ser. No. 15/518,179, filed Apr. 10, 2017, which is a national stage of International Application No. PCT/US2015/54877, filed Oct. 9, 2015, which claims priority to U.S. Provisional Application No. 62/062,460, filed Oct. 10, 2014, the contents of each of which is herein incorporated by reference in their entirety.

### FIELD OF THE INVENTION

This invention relates to methods of analyzing glycans and glycan-binding entities. The invention further provides methods for developing anti-glycan antibodies, arrays and assays for therapeutic, diagnostic and other related purposes.

### BACKGROUND OF THE INVENTION

The synthesis and association of sugar molecules with a variety of structures, including proteins and lipids, occurs throughout nature. Glycobiological studies and characterization have led to an advanced understanding of the role of glycosylation and glycation in a variety of biological processes and disease. Glycans have been shown to be involved in countless processes and pathways including cellular recognition, adhesion as well as numerous signaling pathways and processes (Blixt et al., 2004. PNAS. 101(49):17033-8).

Given the importance of glycans in health and disease, the development of methods and tools for the analysis and characterization of glycans as well as glycan-interacting proteins has been a top priority for those in the field of glycobiology. One such tool is the glycan array. Glycan arrays typically comprise multiple glycans in association with a substrate. In 2002, the use of glycan arrays for the detection and characterization of glycan-interacting agents was described by several groups (Paulson, J. C. et al., Annu Rev Biochem. 2011. 80: 797-823). The technological advances that led to glycan array technology were made possible by advances in the parallel fields of nucleotide and protein chemistry where solid-phase synthesis techniques were first developed (Mrksich, M. Chem Biol. 2004. 11, 739-40). Glycan libraries began to be synthesized using synthetic as well as enzyme-based methods by different groups. Depending on the application for which specific glycan arrays are being developed, different glycan library members or groups of members may be selected and incorporated.

Despite advances in glycan array technology, the enormous complexity of glycans and the complexity of their interactions with various agents continues to limit the scope of glycan array analysis. For instance, glycan conformations may vary greatly under different physiological conditions and/or depending on the structure and density of surrounding glycans. Further, there remains a need in the field for well-defined glycan libraries that are optimized for various applications from specific to broad. Embodiments of the present invention address these limitations with methods, arrays and/or assays described herein.

### SUMMARY OF THE INVENTION

In some embodiments, the present invention provides glycan arrays. These glycan arrays may be comprised of a substrate and at least four glycans wherein from 25% to 75% of the glycans comprise N-acetylneuraminic acid (Neu5Ac). These glycans may be selected from any known glycans, including those described herein. In some cases, glycan arrays comprise from about 30% to about 50% N-glycolylneuraminic acid (Neu5Gc). In some cases, glycan arrays comprise at least one pair of glycans differing only by the substitution of a Neu5Gc residue for a Neu5Ac residue. Some glycan arrays of the invention comprise at least 40 pairs of glycans, each pair differing by the substitution of a Neu5Gc residue for a Neu5Ac residue. Glycans may be linked to arrays by linkers, in some cases selected from —O(CH₂)₂CH₂NH₂ and —O(CH₂)₃NHCOCH₂(OCH₂CH₂)₆NH₂.

In some embodiments, the present invention provides methods of obtaining an anti-glycan antibody profile in a sample comprising contacting a glycan array with a sample, obtaining glycan array binding results and preparing an anti-glycan profile based on the glycan array binding results. Such methods may further comprise selecting at least one binding assay, contacting the sample with the binding assay(s), obtaining results and updating the anti-glycan antibody profile based on the results. In some cases, binding assays are selected from alternative glycan arrays, enzyme-linked immunosorbent assays (ELISAs), flow cytometry-based assays and surface plasmon resonance (SPR)-based assays. These binding assays may be used to assess binding to a modified epitope, such as a chemically modified epitope. Such modified epitopes may include modified saccharides. In such cases, modified saccharides may comprise one or more modified chemical groups.

In some embodiments, the present invention provides a method of obtaining a glycan profile for a sample comprising contacting an array with a sample, obtaining array binding results, and preparing a glycan profile based on the array binding results. Such methods may further comprise selecting at least one other binding assay, analyzing the sample with the binding assay(s), obtaining results, and updating the glycan profile based on those results from the other binding assay(s) (e.g. an alternative array, an ELISA, a flow cytometry-based assay and a SPR-based assay.) In some cases, such binding assays may include anti-glycan antibody arrays. Some binding assays may assess binding to a modified epitope, such as a chemically modified epitope (e.g. a saccharide with one or more chemical groups).

Samples being analyzed may be from in vitro or in vivo sources. In vivo sources may include human subjects and non-human animal subjects. Non-human animal subjects may include mice, rats, rabbits, cats, dogs, pigs, cows, sheep, chicken and monkeys. Samples may be blood, plasma, serum, cells, tissues, organs, mucus, cerebrospinal fluid, saliva and urine.

In some embodiments, the present invention provides methods of diagnosing a disease, disorder and/or condition comprising the use of an anti-glycan antibody profile or a glycan profile according to the present invention. Such diseases, disorders and/or conditions may be cancer or cancer-related indications, immune-related indications, viral indications, cardiovascular indications and/or gastrointestinal indications. Methods of diagnosing cancer or cancer-related indications may comprise the use of anti-glycan antibody profiles comprises anti-tumor associated carbohydrate antigen (TACA) antibody profiles.

In some embodiments, the present invention provides a diagnostic kit comprising one or more glycan arrays of the invention and instructions for use. In some cases, such kits may be used to detect one or more anti-glycan antibodies in a sample.

According to some embodiments, the present invention provides a method of preparing a diagnostic array comprising: (1) obtaining a glycan profile of a cancerous tissue; (2) selecting at least one glycan based on the glycan profile; (3) preparing a pH-optimized printing buffer, wherein the pH of the pH-optimized printing buffer stabilizes at least one chemical group on the selected glycan(s); and (4) preparing a diagnostic array with the glycan(s) and the pH-optimized printing buffer. In some cases, the chemical group is a 9-O acetyl group.

In some embodiments, the present invention provides a method of preparing a diagnostic array comprising: (1) obtaining a glycan profile of a cancerous tissue, wherein the glycan density of the cancerous tissue glycans is determined; (2) selecting at least one cancerous tissue glycan based on the glycan profile; (3) preparing a glycan density-optimized printing buffer; and (4) preparing a diagnostic array with the glycan density-optimized printing buffer. In some cases, the cancerous tissue glycan includes STn.

In other embodiments, the present invention provides a method of diagnosing cancer in a subject comprising: (1) obtaining a subject sample; (2) applying the subject sample to a diagnostic array; and (3) detecting at least one anti-glycan antibody using the diagnostic array, thereby diagnosing cancer. In some cases, the detected anti-glycan antibody is an anti-STn antibody.

### BRIEF DESCRIPTION OF THE FIGURES

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIGS. 1A-1D are diagrams depicting α2,6-sialylated N-acetylgalactosamine (STn) and indicating putative epitopes involved in anti-STn antibody binding. The largest ellipse in each diagram indicates the specific region of STn targeted by each of 4 antibody groups. These groups include Group 1 antibodies (binding to the large elliptical region indicated in FIG. 1A), Group 2 antibodies (binding to the large elliptical region indicated in FIG. 1B), Group 3 antibodies (binding to the large elliptical region indicated in FIG. 1C) and Group 4 antibodies (binding to the large elliptical region indicated in FIG. 1D).

### DETAILED DESCRIPTION

### Introduction

50% or more of proteins are glycosylated. Different organisms, species and even individuals of the same species may comprise different sugars, glycans, glycoproteins, glycolipids and/or other glycosylated structures. Additionally, cellular glycosylation and/or glycosylation patterns may be altered in disease. Such alterations may provide excellent diagnostic and/or therapeutic targets.

In some embodiments, the present invention provides tools for the characterization, detection and/or quantification of biological agents comprising glycans or modified by glycosylation.

### Glycans

As used herein, the terms “glycan”, “oligosaccharide” and “polysaccharide” are used interchangeably and refer to polymers made up of sugar monomers, typically joined by glycosidic bonds also referred to herein as linkages. Within a glycan, monosaccharide monomers may all be the same or they may differ. Common monomers include, but are not limited to trioses, tetroses, pentoses, glucose, fructose, galactose, xylose, arabinose, lyxose, allose, altrose, mannose, gulose, iodose, ribose, mannoheptulose, sedoheptulose and talose. Amino sugars may also be monomers within a glycan. Glycans comprising such sugars are herein referred to as aminoglycans. Amino sugars, as used herein, are sugar molecules that comprise an amine group in place of a hydroxyl group, or in some embodiments, a sugar derived from such a sugar. Examples of amino sugars include, but are not limited to glucosamine, galactosamine, N-acetylglucosamine, N-acetylgalactosamine, sialic acids (including, but not limited to, N-acetylneuraminic acid and N-glycolylneuraminic acid) and L-daunosamine.

Glycans of the present invention may include any of those known in the art. Such glycans may include any of those disclosed by U.S. Pat. Nos. 5,700,916, 5,780,603, 6,972,172, 6,994,966, 7,838,634, 8,119,357 and 8,507,660 as well as by US Publication Nos. US2008/0220988, US2007/0059769, US2004/0259142, US2011/0085981, US2009/0275484 and US2013/0288928, the contents of each of which are herein incorporated by reference in their entirety. Further glycans may include any of those from databases known to those in the field. Such databases may include, but are not limited to the Consortium for Functional Genomics (CFG) mammalian glycan array reagent bank, the CarbBank database and the Glycominds Ltd. seed database.

Glycans may be categorized according to a number of different criteria. Categories may include, but are not limited to sub-structure categories, molecular weight categories, composition categories (e.g. by specific number and type of monosaccharide residues) and linear nomenclature categories.

In some cases, glycans may be modified with one or more non-glycan components including, but not limited to labels, spacers and linkers.

“Sialoglycans,” as referred to herein, are any glycans comprising one or more sialic acid residue. Some proteins are known to be rich in sialic acid. Mucins are one such family of proteins with heavy glycosylation typically comprising high levels of sialoglycans, depending on the where they are synthesized.

### Tumor-Associated Glycans

Aberrant glycosylation is a hallmark of cancer cell transformation. Multiple aberrant glycosylation forms have been described in human cancers and cancerous tissues, identifying specific glycans as a class of cell surface molecules suitable for specific tumor targeting (Cheever, M. A. et al., Clin Cancer Res. 2009 Sep. 1; 15(17):5323-37). Such glycans are referred to herein as “tumor-associated carbohydrate antigens” or “TACAs.” TACA antigen expression has been found in epithelial cancers including, but not limited to, breast, colon, lung, bladder, cervical, ovarian, stomach, prostate, and liver. TACA antigen expression has been found in embryonal cancers including, but not limited to, yolk sac tumors and seminomas. In addition, TACA antigen expression has been found in many melanomas, carcinomas, and leukemias of various tissues (Heimburg-Molinaro et al., Vaccine. 2011 Nov. 8: 29(48):8802-8826).

MUC1 is a key cell surface glycoprotein that is normally extensively glycosylated but is underglycosylated in tumor cells. Sparse glycosylation of MUC1 leads to exposure of immunogenic antigens. These may be along the MUC1 core peptide sequence or along core carbohydrate residues. These TACAs include, but are not limited to N-acetylgalactosamine (Tn), sialyl(α2,6)N-acetylgalactosamine (STn) and galactose(β1-3)N-acetylgalactosamine (also known as Thomsen-Friedenreich antigen or TF). It has been estimated that about 80% of all carcinomas express Tn among the core carbohydrates of MUC1 with STn being strongly expressed on human carcinoma cells and linked to cancer progression and metastasis. With few exceptions, Tn and STn are not expressed in normal healthy tissues. Sialic acid forms a prominent epitope on STn. Interstingly, aberrant Neu5Gc-STn (GcSTn) glycan expression appears to be highly specific to various carcinomas. In the case of MUC1, Neu5Gc incorporation into STn yields a tumor-specific target, a site that is an attractive target for antibody-based therapies to treat tumor tissue. To date, Neu5Gc has been detected in glycoconjugates from a number of human cancer tissues including, but not limited to colon cancer, retinoblastoma tissue, melanoma, breast cancer and yolk sac tumor tissue.

Additional antigens comprising glycans have been identified that are expressed in correlation with cancerous tissue (Heimburg-Molinaro, J. et al., Cancer vaccines and carbohydrate epitopes. Vaccine. 2011 Nov. 8; 29(48):8802-26). These tumor-associated carbohydrate antigens include, but are not limited to blood group Lewis related antigens [including, but not limited to Lewis_{Y} (Le_{Y}), Lewis^{X} (Le^{X}), Sialyl Lewis^{X} (SLe^{X}) and Sialyl Lewis^{A} (SLe^{A})], glycosphingolipid-related antigens [including, but not limited to Globo H, stage-specific embryonic antigen-3 (SSEA-3) and glycosphingolipids comprising sialic acid], ganglioside-related antigens [including, but not limited to gangliosides GD2, GD3, GM2, fucosyl GM1 and Neu5GcGM3] and polysialic acid-related antigens.

### Pathogen-Associated Glycans

Pathogens include a wide class of harmful agents including, but not limited to bacteria, viruses and fungi. Many pathogens express glycans that are secreted and/or displayed on their surface. Pathogen associated glycans may in some cases facilitate immune detection of such pathogens. In some cases, pathogen-associated glycans may inhibit or prevent immune detection.

Due to the fact that many pathogens express pathogen-specific glycans, pathogen-associated glycans may be used to detect and/or quantify such pathogens. The diverse glycans on the surface of pathogens are involved in pathogen and host interactions such as attachment of pathogens to host cells and/or modulation of host immune responses.

Glycans in pathogenic bacteria may be used to detect bacterial infection. Bacterial surface glycans may act as virulence factors for pathogenic infection and disease manifestation. Such bacterial surface glycans, for example, may include, polysaccharide capsules that cover the bacterial surface (e.g., hyaluronan capsule in group A Streptococcus; homopolymeric sialic acid capsule in Neisseria Meningitidis; 1,2,9-linked sialic acid in group C Meningococcal capsules; and 1,2,8-linked sialic acid polymers in group B Meningococcal capsules). In other bacteria, such as Gram negative bacteria (e.g. Yersinia pestis, Pseudomonas aeruginosa and Salmonella), the virulence factors include lipopolysaccharides (LPSs), which are major components of the outer membrane and contain a pathogen-associated molecular pattern (PAMP) that can be recognized by the innate immune system. This may stimulate inflammatory responses to clear bacteria. LPSs can interact with the opsonic receptor CD14 and the membrane protein Toll like receptor 4 (TLR4) to initiate the immune signaling process. Many mucosal pathogens such as H. influenza and Neisseria gonorrhoeae produce lipooligosaccharides (LOSs) that contain a recognizable core structure from which one or more monosaccharides or short oligosaccharide chains extend.

Some bacteria contain proteins such as adhesins in their surface that bind to “receptors” present on the surface of host cells. The interaction of adhesins with receptors mediates bacterial attachment. In such interactions, glycans may form hair-like (e.g. Pili from E. coli and Salmonella); proteinaceous fiber like (e.g., Fimbriae from Bordetella pertussis) or surface anchored protein (e.g., Afimbrial adhesin) glycan structures, interacting with glycoconjugates on the surface of host cells.

In addition, some glycans expressed by pathogens can medicate glycan-lectin interactions which play a pivotal role in pathogen invasion, for example, through epithelial barriers. Other glycans associated with pathogenic bacteria include extracellular polysaccharide (EPS), which promote attachment to host surfaces such as the surfaces of ponds and the surfaces of teeth.

Most viruses use glycan components of cell surfaces for viral infection, as is the case in species and tissue tropism. The well-known influenza virus subtypes are defined based on their surface glycoprotein hemagglutinin (H) and neuraminidase (N). Hemagglutinin on human influenza viruses contain terminal sialic acids with 1,2,6 linkage, while hemagglutinin on bird influenza viruses contain terminal sialic acids with 1,2,3 linkages. Other glycans that can mediate viral infection include, but are not limited to, a family of ten viral envelope glycoproteins (e.g., gB, gD, gC) on Herpes Simplex Virus (HSV); an outer envelope glycoprotein gp120 and a transmembrane glycoprotein gp41 on Human Immunodeficiency Virus (HIV); Sialylated glycans in the capsid protein VP1 of human JC polyomavirus (JCV); the glycoprotein capsule of Molluscipoxvirus (MCV); VP1 of Simian Virus (SV40); N-glycans on Ebola virus GP1; N-linked glycans on the E glycoprotein of Dengue Virus; and glycoproteins on Merkel cell polyoma virus.

The glycan components of the fungal cell wall in pathogenic fungi mediate the interaction between pathogenic fungi (e.g., Cryptococcus neofornans, Aspergillus fumigatus, Kluyveromyces lactis, Candida albicans, Paracocidioides brasilienis and Staphylococcus aureus) and host cells. It has been reported that three types of monosaccharides: D-glucose (Glc), N-acetyl-D-glucosamine (GlcNAc) and D-mannose (Man), within the Candida and Saccharomyces are main components of the glycan chains. Other cell envelope glycans from some pathologic fungi may include, but are not limited to, nigeran, chitin, Galactomannan (glycoprotein), Mannan (glycoprotein/glycolipid), gluomannan, glucuronoxylomannan (capsule), galactoxylomannan (glycoprotein), mannoprotein, glucan and sialic acids (e.g., Masuoka, Clin. Microbiol. Rev., 2004, 17, 281-310).

It is known in the art that pathogenic parasites also produce glycan antigens in surface and secreted glycoproteins and glycolipids. Toxoplasma gondii and other apicomplexan parasites (e.g. Plasmodium for malaria; Toxoplasma for Toxoplasmosis; Neospora cattle and Emeria) can produce MIC proteins (e.g., MIC1, MIC4 and MIC13) which contain a microneme adhesive repeat (MAR) domain which contains tandem sialyl LacNAc glycans and can recognize a wide range of sialyl oligosaccharide sequences on host cells. Many parasitic worms (helminth) such as Schistosoma mansoni and other Schistosoma sp. can produce unusual parasite-synthesized glycans which have immunomodulatory effects. Helminth glycans commonly terminate with beta-linked GalNAc, often in the sequence of GalNAcβ1-4GlcNAc (termed the LacdiNAc motif, LDN). Some have unusual sugars such as tyvelose and/or generate unusual modification of sugars, such as the phosphorylcholine (PC) modification of glycans, 2-O-methylation of fucose and 4-O-methylation of galactose (Prasanphanich et al., Front Immuno., 2013, 4, 240). Such parasite glycans could be exploited in the development of vaccines and for the diagnosis of parasitic infection.

### Glycoconjugates

In some embodiments, glycans may comprise glycoconjugates. Glycoconjugates may include, but are not limited to glycoproteins, glycolipids or proteoglycans. The glycans of glycoproteins, glycolipids and proteoglycans are enormously diverse and involved in many physiological processes such as immuno reaction, pathogen-host interactions and inflammation.

Glycoproteins include any proteins that contain covalently attached oligosaccharide chains (glycans). Glycans are attached to glycoproteins in a cotranslational or posttranslational modification, known as glycosylation. Glycoproteins are present in the extracellular space as secreted molecules, cell surface as integral membrane proteins, or inside the cell. During glycosylation, carbohydrates are often linked to polypeptides through N-linked protein glycosylation (N-glycosylation of N-Glycans) on the amide nitrogen on the side-chain of asparagine (Asn) residues; or through O-linked protein glycosylation (O-glycosylation of O-Glycans) on the hydroxyl oxygen on the side-chain of hydroxylysine, hydroxyproline, serine or threonine residues. The sequences and sizes of oligosaccharide chains on glycoproteins are diverse.

As used herein, the term “glycolipid” refers to compounds composed of lipid that are covalently bound to one or more carbohydrate residues or glycans. Attached carbohydrate residues may include galactose, glucose, inositol, or others. Carbohydrate residues and glycans are usually bound to lipids by a glycosidic linkage to a hydrophobic moiety such as an acylglycerol, a sphingoid, a ceramide or a prenyl phosphate. Glycolipids can be categorized into several subtypes including glycoglycerolipids, which are glycolipids containing one or more glycerol residues; glycosphingolipids (GSLs) which are lipids containing at least one monosaccharide residue and either a sphingoid or a ceramide; glycophosphatidylinositol which are glycolipids which contain carbohydrate residues or glycans glycosidically linked to the inositol moiety of phosphatidylinositols (e.g. diacyl-sn-glycero-3-phosphoinositol), inclusive of lyso-species and those with various O-acyl-, O-alkyl-, O-alk-1-en-1-yl- (e.g. plasmanylinositols) or other substitutions on their glycerol or inositol residues; fucoglycosphingolipid; mannoglycosphingolipid; and xyloglycosphingolipid.

Glycolipids are primarily found in cell membranes as membrane components. Glycolipid-enriched membrane domains can be involved in different biological functions such as cell-cell adhesion, receptor mediated signal transduction and as targets for host pathogens and their toxin bindings.

As used herein, the term“proteoglycan” or “PG” refers to proteins that are heavily glycosylated in which the core protein/polypeptide is covalently bound to one or more glycosaminoglycan (GAG) chains. The GAG chains are attached through a tetrasaccharide bridge to serine residues of the core protein. Proteoglycans can be categorized depending on the nature of their glycosaminoglycan chains as chondroitin sulfate (CS)/dermatan sulfate (DS) proteoglycans (CS/DS-PGs)(e.g., decorin, biglycan, versican), heparan sulfate (HS)/chondroitin sulfate (CS) proteoglycans (e.g., testican, perlecan), chondroitin sulfate (CS) (e.g. bikunin, neurocan, aggrecan), herapan sulfate (HS) (e.g. syndecan, glypican) and keratan sulfate (e.g. fibromodulin, lumican). Proteoglycans are major components of the extracellular matrix forming large complexes.

It has been reported that human tumor cells express altered levels of glycoconjugates and/or aberrant glycoconjugates with structural changes of glycan chains. Such tumor specific glycoproteins, glycolipids and/or proteoglycans play vital roles in tumor aggression and metastasis, participating in cell-cell and cell-extracellular matrix interactions that promote tumor cell proliferation, adhesion and migration.

Many glycoforms of various glycoproteins are associated with cancers, such as a cancer-specific glycoform of periostin, preferably including a GlcNAc β(1,6) Man branched/V-linked glycan component; a cancer-specific glycoform of osteoglycin, preferably including GlcNAc β(1,6) Man branched/V-linked glycan component; lysosomal-associated membrane glycoprotein 1 (LAMP-I), and lectin galactosidase soluble binding protein 3 (GALS3BP) (as taught in U.S. Pat. No. 8,623,611, the contents of which are herein incorporated by reference in their entirety).

Altered levels of PGs and structural changes of GAG chains of PGs are also common in many cancer cells. For example, CSPG4 (Chondroitin Sulfate Proteoglycan 4) and other CS/DS-PGs are overexpressed in breast cancer cells. Glycosylphosphatidylinositol-(GPI-) anchored Heparan sulfate proteoglycan (HSPG) glypican-1 is strongly expressed in human breast and pancreatic cancer (see U.S. Pat. No. 7,108,986).

The aberrant and elevated expression of glycolipids has been demonstrated on the surface of different types of cancer cells which show a significant functional role in a number of cellular physiological pathways related to cancer progression. It is known in the art that sialic acid-containing GSLs and gangliosides are highly expressed in many human cancer cells. For instance, disialoganglioside GD2 is highly expressed on neuroblastoma, melanoma, glioma and small cell lung cancer (SCLC) cells (e.g., Mujoo et al., Cancer Res., 1987, 47, 1098-1104) and GD3 is highly expressed in melanomas, as well as neuroectodermal tumors (neuroblastoma and glioma) and carcinomas, including lung, breast, colon, prostate, and ovarian cancers (e.g., Lo et al., Clin Cancer Res., 2010, 16, 2769).

Many pathogens such as bacteria, virus, fungi and parasites interact with glycoconjugates on the surface of host cells as “receptors” for their pathogenic effect. Through host-pathogen interactions, pathogens invade, disseminate, and evade the host immune system to promote their survival in host environments. Many viruses, bacteria and parasites express adhesins that bind to cell surface heparan sulfate proteoglycans (HSPGs) to facilitate their initial attachment and subsequent cellular entry (i.e. promote the infection) (e.g., Rostank and Esko, Infect Immun., 1997, 65, 1-8; and Spillmann, Biochimie, 2001, 83, 811-817). Pathogens usually bind to precise GAG chains and sulfated domains in host glycoconjugates. For example, the sulfated domain in HS mediates Toxoplasma Gondii attachment to Vero cells; and N. caninum tachyzoites binds to sulfated domain in CS (Naguleswaran et al., Int. J Parasitol., 2002, 32, 695-704). Many pathogens subvert HSPGs on host cells during infection. As non-limiting examples, syndecan-1 can interact with pathogenic proteins AnlB, ANIO, hA and Npr599 on Bacillus anthracis, ClnA on Bacillus cereus, ActA on Listeria monocytogenes, LPS (gingipains) on Porphyromonas gingivalis; LasA on Pseudomonas aeruginosa, alpha-toxin and beta-toxin on Staphylococcus aureus, ZmpC on Staphylococcus pneumoniae, and Opa on Neisseria gonorrhoaea; syndecan-4 can interact with pathogenic proteins on Orientia tsutsugamushi and Opa on Neisseria gonorrhoaea; syndecan-2 can interact with gB,gC,gD and VP3 on HSV-1 and -2; perlecan, agrin and syndecan-3 can interact with HIV viruses; HPV interacts with syndecan-1, 4 and 3, and glypican-1; and Glypican-1 can interact with prions (reviewed by Bartlett and Park, Biology of Extracellular Matrix, 2011, 31-64).

In addition to proteoglycans, glycans linked to glycoproteins and glycolipids may mediate host-pathogen interactions. Recently, many particular carbohydrate sequences (patterns) used by different pathogens have been identified. For example, SV40 virus uses a sialoglycolipid ganglioside GM1 as a cell surface receptor for cell entry during viral infection. The receptors on host cells utilized by influenza virus contain glycan sequences that terminate in sialic acid. Sialic acid-containing glycoproteins can bind directly to rotaviruses (Yolken et al., J. Clin. Invest., 1987, 79, 148-154). The physically closer location of carbohydrate moieties of glycolipids make them favorite adhesion receptors for many microbial pathogens. Neutral glycolipids GA1 can bind to a broad spectrum of enteric viral pathogens (e.g., U.S. Pat. No. 5,192,551). Sulfatides, ganglio- and lacto-series glycolipids can be receptors for several generic pathogens, such as Mycoplasmas. As a non-limiting example, human pathogen M. Pneumoniae specifically binds to sulfatide and other sulfated glycolipids such as seminolipid and lactosylsulfatide and that the consensus binding sequence is a terminal Gal(3SO4)β1-residue (as described in U.S. Pat. No. 5,696,000, the contents of which are herein incorporated by reference in their entirety).

### Glycan Libraries

As used herein, the term “glycan library” refers to a group of two or more glycans. Large and/or diverse chemical libraries may be synthesized according to any methods available in the art. Such methods may include any of those described by U.S. Pat. Nos. 5,700,916, 5,780,603, 6,972,172, 6,994,966, 7,838,634, 8,119,357 and 8,507,660 as well as by US Publication Nos. US2008/0220988, US2007/0059769, US2004/0259142, US2011/0085981, US2009/0275484 and US2013/0288928, the contents of each of which are herein incorporated by reference in their entirety. Glycan libraries may be synthesized with enzymatic methods or chemical synthesis. The chemical synthesis may be performed in solution or on a solid support or a combination of both.

In some embodiments, multiple glycosidic linkages are formed in solution. Multiple glycosidic linkages may be formed in one step based on the discovery that the relative reactivity of glycoside residues containing anomeric sulfoxides and nucleophilic functional groups can be controlled. The activation of anomeric sulfoxides with catalytic quantities of an activating agent provides good yields of condensation product under mild conditions. The activating agent may be a strong organic acid such as trifluoromethanesulfonic or triflic acid (TfOH), p-tolunenesulfonic acid (TsOH) or methanesulfonic acis (MsOH). One or more glycosyl donors having alkyl or aryl sulfoxides at anomeric position and one or more glycosyl acceptors are combined in a reaction vessel. The reaction to form multiple glycosidic linkages in solution is initiated by the addition of an effective amount of an activating agent. Glycosyl acceptors may have chemical groups such as one or more hydroxyls and/or other nucleophilic groups such as amines, and/or silyl ether protected hydroxyls. The glycosyl acceptors and donors may be blocked with a protection group, including but not limited to, ether, ester, acetamido, or thioester at one or more positions. Polarity of the solvent used in the reaction may influence the stereochemistry of glycosylation products.

In some embodiments, large libraries of thiosaccharide derivatives are synthesized by reacting a thiosaccharide with a Michael acceptor or an α-halocarbonyl compound to generate a thiosaccharide carbonyl compound. The carbonyl group of thiosaccharide carbonyl compound can optionally be reduced to form alcohol and/or amine thiosaccharide derivatives, which may be further derivatized to generate other thiosaccharide derivatives, such as but not limited to, esters, amides, carbomates, ureas, thiourea, thioesters and thiocarbamates. The Michael acceptor may include, but is not limited to α,β-unsaturated carbonyl compounds. This synthetic method may be carried out in solution or on a solid support. The thiosaccharide may be covalently attached to a solid support by a cleavable or non-cleavable linker. The solid support having a thiosaccharide covalently attached thereto is contacted with a coupling agent selected from a Michael acceptor or an α-halocarbonyl compound to form a thiosaccharide carbonyl compound which is covalently attached to the solid support.

In some embodiments, a glycan library is synthesized on a solid support. Glycans may be immobilized on a solid support non-covalently or via a covalent bond. The immobilization may be site-specific. In one embodiment, a linking compound is bonded to at least one site on a substrate, wherein at least one end of the linking compound is attached to a solid support and at least one end is attached to a glycan. Non-limited examples of linking compounds include an alkyl, an aminoalkyl, a peptide, an amino acid, a protein or a combination thereof. The linking compound may include a plurality of surface groups that can be attached to glycans. In some embodiments, a 3-D array of glycan libraries may be prepared with dendrimer and/or dendron linking compounds, as disclosed in US 20080220988 to Zhou, the contents of which are incorporated herein by reference in their entirety. In some embodiments, each glycan molecule is covalently attached to the solid support via amide or amine groups. In some embodiments, the solid support is a glass slide. The glass slide may be coated with a hydrogel. In some embodiments, the carbohydrate molecules in the glycan library are reducing end-tagged and may be immobilized on the solid support while solubilized in a solvent comprising an aqueous/aliphatic alcohol mixture as disclosed in US 20040259142 to Chai et al., the contents of which are incorporated herein by reference in their entirety. In some embodiments, a glycan array comprises ω-aminoalkylglycan covalently attached to a functionalized substrate via a polymer or copolymer of an acrylic acid derivative. The glycan array may be fluorescently labelled. The glycan array is fabricated by first quantitatively reacting an ω-aminoalkylglycan with an activated polymer of an acrylic acid derivative to provide a glycoconjugated polymer or copolymer of an acrylic acid derivative; and then covalently attaching the glycoconjugated polymer or copolymer of an acrylic acid derivative to a functionalized substrate, as disclosed in US 20130288928 to Bovin et al., the contents of which are incorporated herein by reference in their entirety. The copolymer of an acrylic acid derivative may comprise fluorescein cadaverine as a fluorescent label or lysine or aminated PEG. The functionalized substrate may be an epoxylated and aminated glass or plastic.

In some embodiments, a combinatorial complex carbohydrate library comprising a plurality of addressable complex carbohydrate structures is synthesized by an enzymatic method. A sequence of enzymatic reactions is determined for each complex carbohydrate constituent of the library. A non-limiting list of enzymatic reactions including donors, acceptors and indexes is shown in Table 7 of U.S. Pat. Nos. 6,972,172 and 6,994,966 to Dukler et al., the contents of each of which are incorporated herein by reference in their entirety. The method may be conducted on a solid support and may comprise a) providing a solid support having a plurality of locations; b) enzymatically synthesizing a plurality of complex carbohydrate structures, each of the plurality of complex carbohydrate structures being attached to at least one addressed location of the plurality of locations, thereby producing the addressable combinatorial complex carbohydrate library. The complex carbohydrates may be attached to the solid support via a linker that can be cleaved under conditions that are harmless to the carbohydrates. The linker may react with a p-nitrophenyl, amine or squaric acid derivative of a sugar and may be selected from an amino acid, a peptide, a non-glycosylated protein, a lipid, a ceramide dolicol phosphate, a cyclodextrin, an oligosaccharide, a monosaccharide, an alkyl chain and a nucleic acid. The link may be at least 20 angstrom in length. The solid support may be selected from addressable microparticles, addressable beads, and a flat platform. The flat platform may be selected from a microtiterplate, a membrane and a chip. Any enzymes capable of synthesizing glycosidic bonds may be used in this method, including but not limited to enzymes in Tables 2, 3 and 5 of U.S. Pat. Nos. 6,972,172 and 6,994,966 to Dukler et al., the contents of each of which are incorporated herein by reference in their entirety. Undesired polymerization may be prevented by using a modified glycosyl donor and a glycosyltransferase with a modified donor specificity. The modifying group may be selectively removed by either an enzymatic or chemical reaction. Any suitable saccharide modifying group may be used, such as but not limited to modifying groups in Table 6 of U.S. Pat. Nos. 6,972,172 and 6,994,966 to Duklar et al., the contents of which are incorporated herein by reference in their entirety.

In some embodiments, the synthesis of glycan library comprises stereospecific steps. As a non-limiting example, the glycan library may comprise sialosides with α-glycosidic linkages such as Neu5Ac. Enzymatic sialylation provides stereo-specific α-linked sialosides and may be used to synthesize a glycan library of naturally occurring sialosides. Various sialic acid donors for efficient α-sialylation have been developed, using leaving groups such as but not limited to halides, phosphites, sulfides, xanthates, phenyltrifluoroacetimidates. Wu et al. teaches an N-acetyl-5-N,4-O-carbonyl-protected dibutyl sialyl phosphate donor for sialylation of both primary and sterically hindered secondary acceptors to prepare sialosides with high yield and α-selectivity, as disclosed in U.S. Pat. No. 8,507,660 to Wu et al., the contents of which are incorporated herein by reference in their entirety. The dibutyl sialyl phosphate donor may be synthesized by coupling a thiosialoside with a dibutyl phosphate in the presence of N-iodosuccinimide and catalytic trifluoromethanesulfonic (triflic) acid under suitable conditions. A library comprising a plurality of sialyl polysaccharides may be synthesized from sialyl disaccharide building blocks generated by coupling the N-acetyl-5-N,4-O-carbonyl-protected dibutyl sialyl phosphate donor with a suitable acceptor.

Glycoprofiling

As used herein, the term “glycoprofiling” includes any analysis that characterizes one or more glycan-related property of a sample or subject. In some cases, glycoprofiling may be carried out to assess the identity, presence and/or absence of one or more glycans associated with one or more proteins or peptides in a sample and/or subject. In some cases, glycoprofiling may be carried out to assess the presence, absence, type, amount and/or specificity of specific anti-glycan antibodies in a sample or subject. In other cases, glycoprofiling may be carried out to identify and/or characterize anti-glycan antibody binding partners.

### Anti-Glycan Antibody Profiling

In some embodiments, glycoprofiling, according to the present invention involves anti-glycan antibody profiling. As referred to herein, anti-glycan antibodies include any antibodies that bind to a glycan or glycoprotein epitope comprising at least one glycan. Anti-glycan antibody profiling may be used to develop an anti-glycan antibody profile for a sample and/or subject. As used herein, an “anti-glycan antibody profile” refers to a set of data, a report or other information format that provides a characterization of the presence, absence, type, amount and/or specificity of anti-glycan antibodies present in a sample and/or subject. In some cases, anti-glycan antibody profiling may be carried out in order to select one or more antibodies from a sample and/or subject to be utilized in further analysis and/or antibody development. In other cases, anti-glycan antibody profiling may be used to analyze antibodies produced by one or more hybridoma cells. Such profiling may be used to select hybridoma cells for clonal expansion and further antibody development.

Anti-glycan antibody profiling in some cases, comprises the use of one or more assays Such assays may include, but are not limited to binding assays, immunological assays, glycan arrays, ELISAs, flow cytometry-based assays and SPR-based assays. Glycan arrays, including any of those described in the current application, may comprise an array of various glycans. Samples may be applied to such arrays to identify and/or characterize antibodies capable of interacting with the distinct glycans on the arrays. In some cases, glycans included in such arrays may be chemically modified to alter one or more chemical groups to alter the profile of antibodies that may bind.

In some cases, anti-glycan antibody profiling may include three-dimensional assessment of antibody-epitope interactions. According to such methods, antibody bound to a particular glycan may be analyzed by a method of three-dimensional assessment, including, but not limited to X-ray crystallography.

In some embodiments, anti-glycan antibody profiling according to the invention may comprise profiling of one or more anti-glycan antibody subsets. Examples of such subsets may include, but are not limited to anti-sialoglycan antibody profiling, anti-TACA antibody profiling, anti-pathogen glycan antibody profiling (e.g. anti-bacterial glycan antibody profiling and anti-viral glycan antibody profiling) and autoimmune anti-glycan antibody profiling. Anti-sialoglycan antibody profiling refers to anti-glycan antibody profiling used to generate an anti-glycan antibody profile specifically characterizing the presence, absence, type, amount and/or specificity of anti-glycan antibodies capable of interacting with one or more sialoglycans. In some cases, anti-sialoglycan antibody profiling may be carried out to characterize the presence, absence, type, amount and/or specificity of anti-glycan antibodies capable of interacting with one or more sialoglycans comprising Neu5Gc.

Methods of the present invention may include anti-TACA antibody profiling. Anti-TACA antibody profiling may be carried out to characterize the presence, absence, type, amount and/or specificity of anti-glycan antibodies capable of interacting with one or more TACA. In some cases, anti-TACA antibody profiling may be used to identify one or more anti-TACA antibodies in a sample. Such samples may include one or more samples taken from a subject suffering from or suspected of having one or more forms of cancer. Anti-TACA antibody detection and/or characterization in such samples may be used to detect and/or diagnose one or more forms of cancer. In some cases, anti-TACA antibody profiling may be used to analyze antibodies present in cell culture medium from one or more hybridoma cells developed for the production of anti-TACA antibodies. Such profiling may be used to select one or more clones for continued development.

In some embodiments, methods of the invention may include anti-pathogen glycan antibody profiling. Pathogens may express characteristic glycans that allow for immune targeting and/or evasion. Anti-glycan antibody profiling may be used to identify, characterize and/or quantify one or more antibodies in a sample capable of binding a pathogen-associated glycan. Such profiling of a subject sample may be used to detect and/or diagnose one or more pathogen-associated diseases, disorders and/or conditions.

### Glycan Profiling

Glycoprofiling methods of the present invention may be used to obtain a glycan profile for a given entity or sample. As used herein the term “glycan profile” refers to a set of data, a report or other information format that provides identifying features of one or more glycans associated with a sample, glycoprotein, cell, tumor and/or tissue. Data from any assays that may be used to identify and/or characterize glycans in a sample may be included in a glycan profile. In some cases, glycan profiles may include, as non-limiting examples, binding assay data, immunological assay data, ELISA data, glycan array data, flow cytometry data, Western Blot data, surface plasmon resonance (SPR) data, enzyme activity data, mass spectrometry data, X-ray crystallographic data and genetic data. Glycosylated samples may include, but are not limited to proteins, cells, cell membranes, tissues, organs and fluids. In some cases, a glycan profile comprises data related to the quantity of one or more glycans in a sample. Some glycan profiles may comprise data related to the identity of glycans in a sample including the percentage of a particular glycan or glycan variant in relation to the total level of glycans or in relation to the level of a particular class or type of glycan. A glycan profile may include glycoprofiling data related to the characterization and/or identity and/or number of chemical groups associated with particular glycans in a sample and/or data characterizing and/or identifying any modifications associated with one or more glycans in a sample.

### Glycoprotein Profiling

As used herein the term “glycoprotein” refers to a protein associated with at least one glycan. Glycoproteins may comprise one or more sites of glycosylation, each of which may fully or partially comprise a diverse arrangement of structurally varied glycans. As a result, isolated glycoprotein samples typically comprise a set of variants with different glycosylation forms, referred to herein as “glycoforms.” In some cases, different glycoprotein glycoforms may have altered functional properties that may ultimately lead to altered health outcomes. In some cases, tumor cells or virally infected cells may express particular glycoforms distinct from glycoforms expressed by healthy cells. This makes methods of identifying and characterizing glycoforms important diagnostic and/or prognostic tools. In some cases, glycan profiles may comprise glycoprotein profiles. As used herein the term “glycoprotein profile” refers to a glycan profile comprising a set of data, a report or other information format that provides characterization, quantification and/or identification information related to one or more glycans associated with one or more proteins or protein glycoforms.

Glycoprotein characterization may include determining the identity of one or more glycans associated with a protein. In some cases, a glycoprotein profile may comprise information related to the identity and/or number of chemical groups associated with particular glycans present on a protein or set of proteins. Some glycoprotein profiles may comprise information on modifications associated with one or more glycoproteins.

In some cases, glycoprotein profiles may comprise a set of data, a report or other information format that identifies a set of glycoforms within a glycoprotein sample. Some glycan profiles may comprise data related to the percentage or ratio of a particular glycoform in relation to the total level of glycoproteins or in relation to the level of a glycoprotein class or type of glycoprotein. In some cases, glycoprotein profiles present information characterizing glycans associated with a particular glycoform and/or provides identifying features of one or more epitopes of a glycoprotein or glycoform of a glycoprotein.

In some embodiments, glycoprotein profiles may be used to evaluate a particular antigen being developed for immunization.

In some cases, a glycoprotein profile may be used to evaluate one or more tumor cells or tissues. Tumor cells may express unique glycoproteins that may be useful as therapeutic targets for antibody development. A glycoprotein profile providing analysis of glycoproteins associated with such tumors may be used to inform development of compounds (e.g. therapeutic antibodies) to combat such tumor cells.

### Glycoprofiling Methods and Uses

### Binding Assays

Glycoprofiling according to the present invention may include the use of one or more binding assays. Binding assays, as referred to herein, include any assays used to determine whether or not two or more entities are capable of forming a bond and/or for determining and/or characterizing the affinity between two or more entities. Affinity may be presented in terms of the dissociation constant between entities, K_{D}, which is a measure of the ratio of dissociated entities to associated entities. A higher K_{D} indicates a weaker bond, while a smaller K_{D} represents a stronger bond. As used herein, K_{D} values are typically presented in molar (M) units indicating the molar concentration of an entity necessary to occupy half of the binding sites available on one or more binding partners. In some cases, affinity may be determined between an antibody and a binding partner (e.g. protein, glycoprotein or glycan) or between an antibody and one or more epitopes on such binding partners. Binding assays of the invention may include, but are not limited to arrays, immunological assays [e.g. enzyme-linked immunosorbent assays (ELISAs,) immunohistochemical assays, radioimmunoassays and immunoprecipitation assays,] flow cytometry-based assays, yeast two-hybrid-based assays and surface plasmon resonance-based assays.

Entities being analyzed by binding assays may include any protein, glycan, glycoprotein, molecule, nucleic acid, antibody, antibody fragment, cell or tissue. Further, other terms that may be used for entities involved in binding assays include probes (e.g. glycan probes,) components, biomarkers, ligands and sensors.

In some binding assays, interactions between entities may be detected through the use of one or more detectable label. Detectable labels may be directly associated with an entity being examined or in some cases, detectable labels may be associated with a secondary agent (e.g. a secondary or detection antibody) capable of associating with one or more of the entities subject to analysis.

In some embodiments, binding assays comprise the anchoring or tethering of a first entity, or probe, to a surface or substrate followed by exposure of the first entity with a second entity being examined for its ability to bind and/or for its level of affinity for the first entity. With such binding assays, second entities may be directly associated with a detectable label. In other cases, a secondary agent, associated with a detectable label, is introduced subsequently to exposure of the first entity with the second entity.

In some cases, probes used in binding assays may comprise glycan probes. Such probes may comprise glycans associated directly with a surface or substrate or may comprise glycans attached to a surface or substrate with a linker.

Linkers useful for tethering entities or probes to a surface or substrate may comprise 10, 11, 12, 13, 14, 15 or more atoms. In a further embodiment, a linker may comprise a group of atoms, e.g., 10-1,000 atoms. Such atoms or chemical groups of atoms may include, but are not limited to, carbon atoms, amino groups, alkylamino groups, oxygen atoms, sulfur atoms, sulfoxide groups, sulfonyl groups, carbonyl groups and imine groups. In some embodiments, linkers may comprise an amino acid, peptide, polypeptide or protein. In some embodiments, a moiety bound by a linker may include, an atom, a chemical group, a nucleoside, a nucleotide, a nucleobase, a sugar, a nucleic acid, an amino acid, a peptide, a polypeptide, a protein, a protein complex, a payload (e.g., a therapeutic agent) or a marker (including, but not limited to a chemical, fluorescent, radioactive or bioluminescent marker). Linkers can be used in the present invention in a variety of applications, such as to form multimers or conjugates, as well as to administer a payload, as described herein. Examples of chemical groups that can be incorporated into linkers include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of selectively cleavable bonds include amido bonds which may be cleaved for example by using tris(2-carboxyethyl)phosphine (TCEP), or other reducing agents, and/or photolysis, as well as ester bonds which may be cleaved for example by acidic or basic hydrolysis. In some embodiments, linkers are carbohydrate moieties. Such carbohydrate linkers may include, but are not limited to —O(CH₂)₂CH₂HN₂ and —O(CH₂)₃NHCOCH₂ (OCH₂CH₂)₆NH₂.

According to the present invention, linkers used to attach entities or probes (e.g. glycan probes) to surfaces or substrates may include any of those known to those of skill in the art, including any of those taught in U.S. Pat. Nos. 6,972,172, 6,994,966, 8,119,357 and 8,507,660, International Publication Nos. WO2013151649 and WO2011088385, the contents of each of which are herein incorporated by reference in their entireties. Linkers may also include Linker-01, Linker-02 or Linker-03 as described in Padler-Karavani et al., 2012. JBC. 287(27): 22593-608, the contents of which are herein incorporated by reference in their entirety.

Glycan probes of the present invention may comprise any glycans. In some cases, glycan probes may include any of those known in the art, including any of those disclosed by U.S. Pat. Nos. 5,700,916, 5,780,603, 6,972,172 (e.g. any of the glycans listed in Tables 5, 9, 10, 12 and 13,) 6,994,966, 7,838,634, 8,119,357 and 8,507,660 as well as by US Publication Nos. US2008/0220988, US2007/0059769 (e.g. any of those depicted in FIG. 2 or FIG. 7 or any of those presented in Table 3 or Table 9,) US2004/0259142, US2011/0085981, US2009/0275484 and US2013/0288928, the contents of each of which are herein incorporated by reference in their entirety. Further glycan probes of the invention may include any of those listed in Tables 1 and/or 2 in Padler-Karavani et al., 2012. JBC. 287(27): 22593-608, the contents of which are herein incorporated by reference in their entirety.

In some cases, array glycans of the invention may include, but are not limited to any of those listed in Table 1.

TABLE 1
Glycan target antigens
Glycan
Araα1,2Araα-R
Araα1,2Glcβ-R
Araα1,3Glcβ-R
Araα1,4Glcβ-R
Araα1,5Araα-R
Araα1,6Glcβ-R
Fucα1,2[Galβ1,4]GlcNAcα-R
Fucα1,2[Galβ1,4]GlcNAcβ-R
Fucα1,2[Galβ1,4]GlcNAcβ-R
Fucα1,2[Galβ1,4]Glcβ-R
Fucα1,2Galβ1,3GlcNAcβ1,3Galβ-R
Fucα1,2Galβ1,3GlcNAcβ-R
Fucα1,2Galβ1,4[Fucα1,3]GlcNAcβ-R
Fucα1,2Galβ1,4GlcNAcβ1,3Galβ-R
Fucα1,2Galβ1,4GlcNAcβ-R
Fucα1,2Galβ-R
Fucα1,3[Fucα1,2Galβ1,4]GlcNAcβ-R
Fucα1,3[Galβ1,4]GlcNAcβ1,3Galβ-R
Fucα1,3[Galβ1,4]GlcNAcβ1,6Galβ-R
Fucα1,3[Galβ1,4]GlcNAcβ-R
Fucα1,3[GlcNAcβ1,3Galβ1,4]GlcNAcβ-R
Fucα1,3GlcNAcβ1,3Galβ1,4Glcβ-R
Fucα1,3GlcNAcβ1,3Galβ-R
Fucα1,3GlcNAcβ1,6[GlcNAcβ1,3]Galβ-R
Fucα1,3GlcNAcβ1,6Galβ-R
Fucα1,3GlcNAcβ1,6Galβ1,4Glcβ-R
Fucα1,3GlcNAcβ-R
Fucα1,3Glcβ-R
Fucα1,4[Galα1,3]GlcNAcβ1,3Galβ-R
Fucα1,4[Galβ1,3]GlcNAcβ1,3Galβ-R
Fucα1,4[Galβ1,3]GlcNAcβ-R
Fucα1,4GlcNAcβ1,3Galβ1,4Glcβ-R
Fucα1,4GlcNAcβ1,3Galβ-R
Fucα1,4GlcNAcβ-R
Fucα1,6[GlcNAcβ1,4]Manα-R
Fucα1,6[Manβ1,4GlcNAcβ1,4]GlcNAcβ-R
Fucα1,6GlcNAcβ-R
Fucβ1,4GlcNAcβ1,3Galβ-R
GalNAcα1,3[Fucα1,2]Galβ1,4-R
GalNAcα1,3[Fucα1,2]Galβ-R
GalNAcα-R
GalNAcβ1,3Galβ1,4Galβ1,4Glcβ-R
GalNAcβ1,4[Neu5Acα2,3]Galβ1,4GlcNAcβ-R
GalNAcβ1,4Galβ1,4Glcβ-R
Galα1,2Galα-R
Galα1,3[Fucα1,2]Galβ1,4-R
Galα1,3Galα-R
Galα1,3Galβ1,4GlcNAcβ-R
Galα1,6Galα-R
Galβ1,2Galβ-R
Galβ1,3GalNAcβ-R
Galβ1,3Galβ1,4Xylβ-R
Galβ1,3Galβ-R
Galβ1,3GlcNAcα-R
Galβ1,3GlcNAcβ1,3Galβ1,4Glcβ-R
Galβ1,3GlcNAcβ1,3Galβ-R
Galβ1,3GlcNAcβ1,6Galβ1,4Glcβ-R
Galβ1,3GlcNAcβ-R
Galβ1,4[Fucα1,3]GlcNAcβ-R
Galβ1,4GlcNAc1,4[GlcNAcβ1,2]Manα-R
Galβ1,4GlcNAc6SB-R
Galβ1,4GlcNAcβ1,3Galβ1,4GlcNAcβ-R
Galβ1,4GlcNAcβ1,3Galβ1,4Glcβ-R
Galβ1,4GlcNAcβ1,3Galβ-R
Galβ1,4GlcNAcβ1,4[GlcNAcβ1,2]Manα-R
Galβ1,4GlcNAcβ1,6Galβ-R
Galβ1,4GlcNAcβ1,6Galβ1,4Glcβ-R
Galβ1,4GlcNAcβ-R
Galβ1,4Glcβ-R
Galβ1,4Xylβ-R
Galβ1,6Galβ-R
Galβ1,6Galβ1,4Gal1,4Glcβ-R
Galβ1,6Galβ1,4Galβ1,4Glcβ-R
GlcAβ1,3Galβ1,3Gal1,4Xylβ-R
GlcAβ1,3Galβ1,3Galβ1,4Xylβ-R
GlcNAcβ1,2Manα1,3[Manα1,6]Manβ-R
GlcNAcβ1,3[Galβ1,6]GlcNAcβ-R
GlcNAcβ1,3[GlcNAcβ1,6]GalNAcβ-R
GlcNAcβ1,3[GlcNAcβ1,6]Galβ-R
GlcNAcβ1,30[GlcNAcβ1,6]Galβ-R
GlcNAcβ1,3GalNAcα-R
GlcNAcβ1,3GalNAcβ-R
GlcNAcβ1,3Galα-R
GlcNAcβ1,3Galβ1,3GalNAcβ-R
GlcNAcβ1,3Galβ1,4GlcNAcβ1,3Galβ-R
GlcNAcβ1,3Galβ1,4GlcNAcβ-R
GlcNAcβ1,3Galβ-R
GlcNAcβ1,4[Fucα2,6]GlcNAcβ-R
GlcNAcβ1,4[Galβ1,4GlcNAcβ1,2]Manα-R
GlcNAcβ1,4[GlcNAcβ1,2]Manα-R
GlcNAcβ1,4GlcNAcα-R
GlcNAcβ1,4GlcNAcβ-R
GlcNAcβ1,6[Galβ1,3]GalNAcβ-R
GlcNAcβ1,6[Galβ1,3]GlcNAcβ-R
GlcNAcp1,6[Galβ1,3GlcNAcβ1,3]Galβ-R
GlcNAcβ1,6[GlcNAcβ1,3]Galβ1,4Glcβ-R
GlcNAcβ1,6GalNAcβ1,3Galα-R
GlcNAcβ1,6Galα-R
GlcNAcβ1,6Galβ-R
GlcNAcβ1,6Galβ1,3GlcNAcβ-R
GlcNAcp1,6Galβ1,4GlcNAcβ-R
Glcα1,2Glcα-R
Glcα1,3Glcα-R
Glcα1,4Glcα-R
Glcα1,6Glcα-R
Glcβ1,2Glcβ-R
Glcβ1,3Glcβ-R
Glcβ1,6Glcβ-R
Glcβ1,6Glcβ-R
KDNα2,8Neu5Acα2,3Galβ1,4Glcβ-R
KDNα2,8Neu5Gcα2,3Galβ1,4Glcβ-R
Manα1,2Manα1,2Manα-R
Manα1,2Manα-R
Manα1,3[Manα1,6]Manβ1,4GlcNAcβ-R
Manα1,3Manα1,2Manα1,2Manα-R
Manα1,3Manα1,4GlcNAcβ1,4GlcNAcβ-R
Manα1,3Manα-R
Manα1,4GlcNAcβ1,4[Fucα1,6]GlcNAcβ-R
Manα1,4GlcNAcβ1,4GlcNAcβ-R
Manα1,6Manα-R
Manα1,6Manα1,4GlcNAcβ1,4GlcNAcβ-R
Manβ1,4GlcNAcβ1,4[Fucα1,6]GlcNAcβ-R
Manβ1,4GlcNAcβ1,4[Fucα2,6]GlcNAcβ-R
Manβ1,4GlcNAcβ1,4GlcNAcβ-R
Manβ1,4GlcNAcβ1,4GlcNAcβ-R
Manβ1,4GlcNAcβ-R
Neu5,9Ac2α2,3Galβ1,3GalNAcα-R
Neu5,9Ac2α2,3Galβ1,3GalNAcβ-R
Neu5,9Ac2α2,3Galβ1,3GlcNAcβ-R
Neu5,9Ac2α2,3Galβ1,4GlcNAcβ-R
Neu5,9Ac2α2,3Galβ1,4Glcβ-R
Neu5,9Ac2α2,3Galβ-R
Neu5,9Ac2α2,6GalNAcα-R
Neu5,9Ac2α2,6Galβ1,4GlcNAcβ-R
Neu5,9Ac2α2,6Galβ1,4Glcβ-R
Neu5,9Ac2α2,6Galβ-R
Neu5Acα2,3Galβ1,3[Neu5Acα2,6]GalNAcα-R
Neu5Acα2,3Galβ1,3GalNAcα-R
Neu5Acα2,3Galβ1,3GalNAcβ-R
Neu5Acα2,3Galβ1,3GlcNAcα-R
Neu5Acα2,3Galβ1,3GlcNAcβ1,3Galβ1,4Glcβ-R
Neu5Acα2,3Galβ1,3GlcNAcβ-R
Neu5Acα2,3Galβ1,4(Fucα1,3)GlcNAc6Sβ-R
Neu5Acα2,3Galβ1,4(Fucα1,3)GlcNAcβ-R
Neu5Acα2,3Galβ1,4[Fucα1,3]GlcNAcβ-R
Neu5Acα2,3Galβ1,4GlcNAc6Sβ-R
Neu5Acα2,3Galβ1,4GlcNAcα-R
Neu5Acα2,3Galβ1,4GlcNAcβ-R
Neu5Acα2,3Galβ1,4Glcβ-R
Neu5Acα2,3Galβ-R
Neu5Acα2,6(KDNα2,3)Galβ1,4Glcβ-R
Neu5Acα2,6(Neu5Acα2,3)Galβ1,4Glcβ-R
Neu5Acα2,6(Neu5Gcα2,3)Galβ1,4Glcβ-R
Neu5Acα2,6GalNAcα-R
Neu5Acα2,6GalNAcα-R
Neu5Acα2,6Galβ1,3GalNAcα-R
Neu5Acα2,6Galβ1,4GlcNAcα-R
Neu5Acα2,6Galβ1,4GlcNAcβ-R
Neu5Acα2,6Galβ1,4GlcNAcβ-R
Neu5Acα2,6Galβ1,4Glcβ-R
Neu5Acα2,6Galβ-R
Neu5Acα2,8KDNα2,6Galβ1,4Glcβ-R
Neu5Acα2,8Neu5Acα2,3Galβ1,4Glcβ-R
Neu5Acα2,8Neu5Acα2,3Galβ-R
Neu5Acα2,8Neu5Acα2,6Galβ1,4Glcβ-R
Neu5Acα2,8Neu5Acα2,8Neu5Acα2,3Galβ1,4Glcβ-R
Neu5Acα2,8Neu5Gcα2,3Galβ1,4Glcβ-R
Neu5Acα2,8Neu5Gcα2,6Galβ1,4Glcβ-R
Neu5Gc9Acα2,3Galβ1,3GalNAcα-R
Neu5Gc9Acα2,3Galβ1,3GalNAcβ-R
Neu5Gc9Acα2,3Galβ1,3GlcNAcβ-R
Neu5Gc9Acα2,3Galβ1,4GlcNAcβ-R
Neu5Gc9Acα2,3Galβ1,4Glcβ-R
Neu5Gc9Acα2,3Galβ-R
Neu5Gc9Acα2,6GalNAcα-R
Neu5Gc9Acα2,6Galβ1,4GlcNAcβ-R
Neu5Gc9Acα2,6Galβ1,4Glcβ-R
Neu5Gc9Acα2,6Galβ-R
Neu5GcOMeα2,8Neu5Acα2,3Galβ1,4Glcβ-R
Neu5Gcα2,3Galβ1,3GalNAcα-R
Neu5Gcα2,3Galβ1,3GalNAcβ-R
Neu5Gcα2,3Galβ1,3GlcNAcβ1,3Galβ1,4Glcβ-R
Neu5Gcα2,3Galβ1,3GlcNAcβ-R
Neu5Gcα2,3Galβ1,4(Fucα1,3)GlcNAc6Sβ-R
Neu5Gcα2,3Galβ1,4(Fucα1,3)GlcNAcβ-R
Neu5Gcα2,3Galβ1,4GlcNAc6Sβ-R
Neu5Gcα2,3Galβ1,4GlcNAcβ-R
Neu5Gcα2,3Galβ1,4Glcβ-R
Neu5Gcα2,3Galβ-R
Neu5Gcα2,6GalNAcα-R
Neu5Gcα2,6Galβ1,4GlcNAcβ-R
Neu5Gcα2,6Galβ1,4Glcβ-R
Neu5Gcα2,6Galβ-R
Neu5Gcα2,8Neu5Acα2,3Galβ1,4Glcβ-R
Neu5Gcα2,8Neu5Gcα2,3Galβ1,4Glcβ-R
NeuAcα2,3Galβ1,3[NeuAcα2,6]GalNAcα-R
Xylα1,2Manα-R
Xylα1,3Glcβ-R
Xylα1,3Xylα1,3Glcβ-R

The following abbreviations are used herein: Glc—glucose, Gal—galactose, GlcNAc—N-acetylglucosamine, GalNAc—N-acetylgalactosamine, GlcNAc6S—6-Sulfo-N-acetylglucosamine, KDN-2-keto-3-deoxy-D-glycero-D-galactonononic acid, Neu5,9Ac2—N-acetyl-9-O-acetylneuraminic acid, Fuc—fucose and Neu5GcOMe—2-O-methyl-N-glycolylneuraminic acid. 0-glycosidic bonds are present between each residue in the glycans listed with α and β indicating the relative stoichiometry between the two residues joined by the bond, wherein α indicates an axial orientation and β indicates an equatorial orientation. The numbers following α and/or β, in the format x,x, indicated the carbon number of each of the carbons from each of the adjoined residues that participate in bond formation. While the glycans listed in Table 1 represent individual glycan probes contemplated, the present invention also includes embodiments wherein the above presented glycans comprise different combinations of α and β-oriented O-glycosidic bonds than the ones presented. Also in Table 1, R represents an entity that the glycan may be coupled with. In some embodiments, R is a protein wherein the glycan is linked typically to a serine or threonine residue. In some embodiments, R is a linker molecule used to join the glycan to a surface or substrate (e.g. as in a glycan array or a carrier protein used in glycan synthesis). In some embodiments, R may be biotin, albumin, ProNH₂, —CH—, —OH, —OCH₃, —OCH₂CH₃, —H, hydrido, hydroxy, alkoxyl, oxygen, carbon, sulfur, nitrogen, polyacrylamide, phosphorus, NH₂, ProNH₂═O(CH₂)₂CH₂NH₂, (OCH₂CH₂)₆NH₂, O(CH₂)₃NHCOCH₂ (OCH₂CH₂)₆NH₂, the fluorescent labels 2-aminobenzamide (AB) and/or 2-aminobenzoid acid (AA), 2-aminobenzamide analog that contains an alkyl amine (AEAB), aminooxy-groups, methylaminooxygroups, hydrazide groups, amino lipid 1,2-dihexadecyl-sn-glycero-3-phosphoethanolamine (DHPE), aminooxy (AO) functionalized DHPE and glycosylphosphatidylinositol (GPI). Without intending to limit the source or nature of R, this may include structures that affect the physical spacing of glycan residues. In some embodiments, the R group may comprise a combination of the R groups presented here, e.g. a biotinylated polyacrylamide. In some embodiments, the R group in combination with underlying substrates effect glycan probe spacing on a surface or substrate.

Glycan probes of the present invention may be purchased commercially or synthesized. In some cases, glycan synthesis may be carried out by enzymatic synthesis.

For sialoglycan probes, synthesis may be carried out according to any methods known in the art. In some cases, the “one-pot three-enzyme chemoenzymatic approach” may be carried out according to the methods described by Yu et al (Yu, H. et al., Nat Protoc. 2006. 1(5): 2485-92, Yu, H. et al., J Am Chem Soc. 2005. 127:17618-9 and Yu, H. et al., 2006. Angew Chem Int Ed Engl. 45:3938-44, the contents of each of which are herein incorporated by reference in their entirety). According to this method, glycoconjugates comprising sialic acid (and their derivatives) are generated through condensation reactions with N-acetylmannosamine, mannose or modified derivatives, catalyzed by sialic acid aldolase. Activation of the resulting compounds is achieved with CMP-sialic acid synthetase. Activated compounds are then transferred to acceptor compounds using sialyltransferases. Sialoglycans are freed from glycoconjugates by treatment with 2 M acetic acid and 3 hours of hydrolysis at 80° C.

Surfaces or substrates useful for anchorage or tethering of an entity in a binding assay may be comprised of a variety of materials and may be used in a variety of shapes, formats and orientations. Surfaces may include the surface of a plate or dish, including, but not limited to a well of a culture dish. In some cases, surfaces may include the inside or outside of a tube or cylinder (e.g. a column). In some cases, surfaces may include the surface of a membrane [e.g. nitrocellulose membrane or polyvinyl difluoride (PVDF) membrane] or filter paper. In some cases, such surfaces include the surface of cells or tissue (including, but not limited to thin sectioned tissues from paraffin embedded or frozen tissue samples).

In some embodiments, binding assays are carried out in array format, where a panel of two or more entities are anchored or tethered to one or more surface or substrate for simultaneous analysis.

### Glycan Arrays

As used herein, the term “glycan array” refers to a binding assay in array format that is used to identify agents that interact with any of a number of different glycans linked to the array substrate (referred to herein as “glycan probes.” In some embodiments, glycan arrays comprise a number of chemically-synthesized glycan probes. In some embodiments, glycan arrays comprise at least 2, at least 5, at least 10, at least 20, at least 30, at least 40, at least 50, at least 60, at least 70, at least 80, at least 90, at least 100, at least 150, at least 350, at least 1000 or at least 1500 glycan probes. In some embodiments, glycan arrays may be customized to present a desired set of glycan probes.

Glycan probes present on arrays of the invention may comprise any glycans. In some cases, glycan probes present on arrays may include two or more of any of those known in the art, including any of those disclosed by U.S. Pat. Nos. 5,700,916, 5,780,603, 6,972,172 (e.g. any of the glycans listed in Tables 5, 9, 10, 12 and 13,) 6,994,966, 7,838,634, 8,119,357 and 8,507,660 as well as by US Publication Nos. US2008/0220988, US2007/0059769 (e.g. any of those depicted in FIG. 2 or FIG. 7 or any of those presented in Table 3 or Table 9,) US2004/0259142, US2011/0085981, US2009/0275484 and US2013/0288928, the contents of each of which are herein incorporated by reference in their entirety. Further glycan probes present on arrays may include any of those listed in Tables 1 and/or 2 in Padler-Karavani et al., 2012. JBC. 287(27): 22593-608, the contents of which are herein incorporated by reference in their entirety.

In some cases, array glycans of the invention may include, but are not limited to any of those listed in Table 1.

In some embodiments, glycan arrays comprise more than 70 chemically-synthesized glycans. In some cases, such arrays may comprise one or more Neu5Ac and Neu5Gc-containing glycan pairs.

Glycan probes used in glycan arrays of the present invention may be purchased commercially or synthesized. In some cases, glycan synthesis may be carried out by enzymatic synthesis as described previously.

### Glycan Array Fabrication

Arrays may be fabricated according to any methods known in the art. Such methods may include, but are not limited to any of those taught by International Publication Nos. WO2013151649 and WO2011088385, U.S. Pat. Nos. 5,700,916, 5,780,603, 6,972,172, 6,994,966, 7,838,634, 8,119,357 and 8,507,660 as well as by US Publication Nos. US2008/0220988, US2007/0059769, US2004/0259142, US2011/0085981, US2009/0275484 and US2013/0288928, the contents of each of which are herein incorporated by reference in their entirety. Further array fabrication may be carried out according to the methods described in Padler-Karavani et al., 2012. JBC. 287(27): 22593-608, the contents of which are herein incorporated by reference in their entirety.

Array substrates may include a variety of materials. In some cases, arrays may be printed on epoxide-derivatized slides. Further, arrays may be printed using any technologies available in the art. In some cases, printing is carried out using a microarrayer device. Such devices may include, but are not limited to microarrayers using linear servo motor technology. Microarrayers of the invention may utilize spotting pins for application of glycans to array substrates. Such spotting pins may include, but are not limited to silicon microarray spotting pins. Spotting pins may comprise pin tips that are from about 10 μm to about 200 μm in size (e.g. from about 10 to about 50, from about 25 to about 75, from about 50 to about 100 and from about 75 to about 200 μm). Spotting pins may also comprise volumes of from about 0.05 μl to about 1 μl (e.g. from about 0.05 to about 0.2, from about 0.1 to about 0.5, from about 0.25 to about 0.75, from about 0.5 to about 1.0 μl). Spotting pins of the invention may be used to generate glycan spots with diameters of from about 1 μm to about 500 μm (e.g. from about 1 to about 10, from about 5 to about 50, from about 20 to about 70, from about 50 to about 100, from about 75 to about 150, from about 100 to about 300, from about 200 to about 500 μm).

Array glycans may be associated with the array substrate via one or more linkers, including any of the linkers described herein. Linkers useful for tethering entities or probes to an array substrate may comprise 1-10, 11, 12, 13, 14, 15 or more atoms. In a further embodiment, a linker may comprise a group of atoms, e.g., 10-1,000 atoms. Such atoms or chemical groups of atoms may include, but are not limited to, carbon atoms, amino groups, alkylamino groups, oxygen atoms, sulfur atoms, sulfoxide groups, sulfonyl groups, carbonyl groups and imine groups. In some embodiments, linkers may comprise an amino acid, peptide, polypeptide or protein. In some embodiments, linkers used to link array glycans to array substrates may comprise —(CH₂)₂CH₂NH₂ or —(CH₂)₃NHCOCH₂(OCH₂CH₂)₆NH₂. In some embodiments, linkers may comprise biotin, albumin, ProNH₂, —CH—, —OH, —OCH₃, —OCH₂CH₃, —H, hydrido, hydroxy, alkoxyl, oxygen, carbon, sulfur, nitrogen, polyacrylamide, phosphorus, NH₂, ProNH₂═O(CH₂)₂CH₂NH₂, (OCH₂CH₂)₆NH₂, O(CH₂)₃NHCOCH₂ (OCH₂CH₂)₆NH₂, the fluorescent labels 2-aminobenzamide (AB) and/or 2-aminobenzoid acid (AA), 2-aminobenzamide analog that contains an alkyl amine (AEAB), aminooxy-groups, methylaminooxygroups, hydrazide groups, amino lipid 1,2-dihexadecyl-sn-glycero-3-phosphoethanolamine (DHPE), aminooxy (AO) functionalized DHPE and glycosylphosphatidylinositol (GPI). Without intending to limit their source or nature, linkers may include structures that affect the physical spacing of glycan residues. In some embodiments, linkers may comprise a combination of any of the linkers presented herein, e.g. a biotinylated polyacrylamide. In some embodiments, the linkers in combination with underlying substrates may affect glycan residue spacing.

In some embodiments, linkers tethering array glycans may comprise a 2-azidoethyl group or N-acetyl-carboxymethyl-threonine. In some embodiments, linkers tethering array glycans may comprise biotin. Such linkers may include -LC-LC-Biotin that may be incorporated via a commercially available kit [e.g. EZ-link kits available from Thermo Scientific (Waltham, MA.]

In some embodiments, linkers may comprise polyacrylamide (PAA). Such linkers may include one or more biotin residues. PAA linkers and or glycans conjugated with PAA linkers may be obtained commercially, for example from GlycoTech (Gaithersburg, Maryland).

Linkers may be varied in order to alter various array properties. In some cases, linkers may be selected to reduce the occurrence of false-negative binding to array glycans. Considerations for making such selections may include those described by Grant et al. (Grant, O. C. et al., Glycobiology. 2014. 24(1):17-25; the contents of which are herein incorporated by reference in their entirety).

### Sialoglycan Arrays

In some embodiments, glycan arrays of the invention may be sialoglycan arrays. As used herein, the term “sialoglycan array” refers to a glycan array with at least one glycan probe comprising one or more sialic acid residues (e.g. Neu5Ac, Neu5Gc or KDN). In some cases, sialoglycan arrays may be used to assess the specificity of one or more anti-glycan antibodies for glycans comprising alternative sialic acid residues. For example, a sialoglycan array with glycan pairs differing only by Neu5Ac vs. Neu5Gc content may be used to determine the importance of specific sialic acid residues for antibody binding and/or to select antibodies based on their ability to differentiate between glycans with one form of sialic acid over another.

Sialoglycan arrays may be characterized in terms of sialic acid presentation ratio. A sialic acid presentation ratio may refer to the ratio of array glycans with one or more sialic acid residue in comparison to the number of array glycans without sialic acid residues. In some cases, the sialic acid presentation ration may refer to the ratio of array glycans with one form of sialic acid residue in comparison to the number of array glycans with an alternative sialic acid residue (e.g. Neu5Ac, Neu5Gc, KDN). In one example, sialoglycan arrays may have a Neu5Ac:Neu5Gc presentation ratio of 25%, where 25% of the array glycans comprise Neu5Ac, while 75% of the array glycans comprise Neu5Gc. In some cases, Neu5Ac:Neu5Gc presentation ratios may be from about 1% to about 99% (e.g. from about 1% to about 10%, from about 5% to about 50%, from about 15% to about 45%, from about 25% to about 75%, from about 30% to about 60%, from about 40% to about 80%, from about 50% to about 75%, from about 70% to about 90%, from about 85% to about 95% or from about 90% to about 99%).

### Anti-Glycan Arrays

Anti-glycan arrays of the invention are arrays comprising one or more glycan-binding agents. As used herein, a “glycan-binding agent” refers to an entity capable of forming a bond with a glycan and/or glycoprotein. Glycan binding agents may include, but are not limited to antibodies, lectins, enzymes (e.g. glycosidases,) small molecules, aptamers and lipids.

Lectins, as referred to herein, are proteins that bind glycans. Lectins are typically plant-derived, but mammalian-derived lectins are encompassed by the term “lectin” as used herein. Lectins useful in aspect of the present invention include, but are not limited to lectins derived from Conavalia ensiformis, Anguilla anguilla, Tritium vulgaris, Datura stramonium, Galnthus nivalis, Maackia amurensis, Arachis hypogaea, Sambucus nigra, Erythtina cristagalli, Sambucis nigra, Erythrina cristagalli, Lens culinaris, Glycine max, Phaseolus vulgaris Allomyrina dichotoma, Dolichos biflorus, Lotus tetragonolobus, Ulex europaeus, and Ricinus commurcis. Other proteins capable of binding glycans may include cell receptors, growth factors, cytokines and extracellular matrix proteins, and thus, for the purposes of this invention, are encompassed by the term “lectin” as used herein.

Glycosidases useful as glycan-binding agents may include, but are not limited to α-glycosidase, 3-galactosidase, N-acetylhexosaminidase, α-mannosidase, β-mannosidase and α-fiucosidase.

In some cases, the present invention provides anti-glycan arrays comprising antibody arrays, where antibodies represent glycan-binding agents in the array. Such arrays may comprise arrays of antibodies, antibody fragments and/or fusion proteins comprising one or more antibody variable domain directed toward one or more glycans or one or more glycan epitopes. In some cases, detection of glycans bound to specific antibodies present on such arrays may be detected through the use of surface plasmon resonance. Such techniques include those described in Houngkamhang, N. et al., 2013. Sensors. 13:11913-22, the contents of which are herein incorporated by reference in their entirety.

Anti-glycan arrays of the present invention may be used to detect and/or identify one or more glycan and/or glycan epitopes present in a particular sample. In some cases, anti-glycan arrays may be used to obtain a glycoprotein profile for a glycoprotein, one or more glycoprotein glycoforms or for a set of glycoforms within a glycoprotein sample.

In some cases, anti-glycan arrays of the present invention may be formatted and utilized according to UC-FINGERPRINT™ analysis methods described in International Publications WO2000/668688, WO2001/84147, WO2002/37106 or WO2002/44714, the contents of each of which are herein incorporated by reference in their entirety. In some cases, anti-glycan arrays of the present invention may be formatted and utilized according to the modified version of the UC-FINGERPRINT™ analysis methods described in U.S. Pat. No. 8,119,357, the contents of which are herein incorporated by reference in their entirety.

In some embodiments, anti-glycan arrays may comprise any of the antibodies (or fragments of such antibodies) described in International Publication No. WO2013151649 or US Publication No. US2014/0178365, the contents of each of which are herein incorporated by reference in their entirety.

### Immunological Assays

Binding assays of the invention may include immunological assays. As used herein the term “immunological assay” refers to any assay that utilizes antibodies or antibody fragments in the detection or characterization of a given entity, including, but not limited to characterization of binding, affinity, concentration, isoform or confirmation. Such entities may include, but are not limited to glycans, proteins, glycoproteins, antibodies, lectins, small molecules, aptamers and lipids.

Immunological assays may include, but are not limited to ELISAs, immunohistochemical assays, radioimmunoassays and immunoprecipitation assays. Further immunological assays may include flow-cytometry-based assays.

ELISAs are routine to those skilled in the art and may be carried out, for example, according to the methods described in International Publication No. WO2013151649 or US Publication No. US2014/0178365, the contents of each of which are herein incorporated by reference in their entirety. ELISAs may comprise “sandwich assays”. As used herein, the term “sandwich assay” refers to an immunological assay wherein factors being detected are bound by at least two antibodies, wherein one antibody captures such factors and another antibody associates only with regions, features or epitopes of such factors with which detection is desired. Such assays typically comprise a capture antibody and a detection antibody. As used herein, the term “capture antibody” refers to an antibody component of an immunological assay, typically bound to a substrate, capable of associating with an antigen or other factor being detected in an assay. Capture antibodies may bind to one or more capture epitope. When referring to factors being detected in a sandwich assay, the term “capture epitope,” as used herein, refers to an epitope that does not comprise regions, features or epitopes of such factors that bind to detection antibodies in such sandwich assays. Association of capture antibodies with one or more capture epitopes holds factors being detected in an orientation that facilitates interaction of such factors with a detection antibody.

As used herein, the term “detection antibody” refers to an antibody component of an immunological assay that associates with one or more detection epitopes. When referring to factors being detected in a sandwich assay, the term “detection epitope” refers to an epitope that comprises regions, features or epitopes of such factors that are being detected in such sandwich assays. Detection antibodies may be associated with one or more detectable labels to facilitate detection and/or quantification of bound antigens. Such labels may include, but are not limited to fluorescent tags, biotin moieties and/or enzymes. Detectable labels comprising enzymes may comprise horseradish peroxidase (HRP).

In some embodiments, sandwich assays of the present invention may comprise secondary detection antibodies. As used herein, the term “secondary detection antibody” refers to an antibody capable of associating with detection antibodies. Secondary detection antibodies may comprise detectable labels. Such labels may include, but are not limited to fluorescent tags, biotin moieties and/or enzymes. Some detectable labels comprising enzymes may comprise HRP.

### Surface Plasmon Resonance (SPR)

In some embodiments, glycoprofiling may comprise the use of surface plasmon resonance technology. Methods of using surface plasmon resonance are well known to those of skill in the art and may be used to assess bond formation and/or affinity between two or more entities. Surface plasmon resonance may be carried out, for example, with a BIAcore 3000 instrument.

In some cases, bond formation between an antibody and a known or suspected ligand may be assessed. Such binding partners may include proteins, glycoproteins, and glycans including different epitopes present on such binding partners. In some cases, these techniques may be used to determine the affinity of an antibody for an antigen used during immunization in the development of the antibody.

### Flow Cytometry

Glycoprofiling according to the present invention may include the use of flow cytometry-based assays. Flow cytometry may be used to assess binding of a given entity to a live cell and methods are well known in the art. Flow cytometry assays according to the present invention may be carried out, for example, as described in International Publication No. WO2013151649 or US Publication No. US2014/0178365, the contents of each of which are herein incorporated by reference in their entirety. Such assays may be useful when evaluating the binding of one or more entities (e.g. antibodies, glycans, proteins or glycoproteins) to a protein, glycoprotein, glycan, glycolipid, proteoglycan or other molecule or complex present expressed on the surface of a cell. Glycans, proteins and/or glycoproteins present on the surface of a live cell may comprise a conformation or three-dimensional arrangement that more closely resembles their conformation or three-dimensional arrangement in vivo. Thus, binding data obtained from flow cytometry-based assays may more closely reflect in vivo interactions. In some cases, flow cytometry may be used to test antibodies being developed to target a glycan or glycoprotein present on the surface of one or more cell types. Some such cell types may be tumor cells with one or more unique glycan or glycoprotein targets expressed on their surface. Some cells used in flow cytometry may include immune cells with glycan or glycoprotein targets expressed on their surface.

### Antibody Development

In some embodiments, methods of the present invention, including glycoprofiling methods as described herein, may be used to develop antibodies. As used herein, the term “antibody” is used in the broadest sense and specifically covers various embodiments including, but not limited to monoclonal antibodies, polyclonal antibodies, multispecific antibodies (e.g. bispecific antibodies formed, for example, from at least two intact antibodies), and antibody fragments such as diabodies so long as they exhibit a desired biological activity. Antibodies are primarily amino-acid based molecules but may also comprise one or more modifications such as with sugar moieties, linkers, detectable labels and the like.

“Antibody fragments” comprise a portion of an intact antibody, preferably comprising an antigen binding region thereof. Examples of antibody fragments include Fab, Fab′, F(ab′)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. Some antibodies of the present invention may comprise one or more of these fragments. For the purposes herein, an “antibody” may comprise a heavy and light variable domain as well as an Fc region.

“Native antibodies” are usually heterotetrameric glycoproteins of about 150,000 Daltons, composed of two identical light (L) chains and two identical heavy (H) chains. Genes encoding antibody heavy and light chains are known and segments making up each have been well characterized and described (Matsuda, F. et al., 1998. The Journal of Experimental Medicine. 188(11); 2151-62 and Li, A. et al., 2004. Blood. 103(12: 4602-9, the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FW) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments can also be used based on comparisons with other antibodies (Strohl, W. R. Therapeutic Antibody Engineering. Woodhead Publishing, Philadelphia PA. 2012. Ch. 3, p 47-54, the contents of which are herein incorporated by reference in their entirety).

VH and VL domains have three CDRs each. VL CDRs are referred to herein as CDR-L1, CDR-L2 and CDR-L3, in order of occurrence when moving from N- to C-terminus along the variable domain polypeptide. VH CDRs are referred to herein as CDR-H1, CDR-H2 and CDR-H3, in order of occurrence when moving from N- to C-terminus along the variable domain polypeptide. Each of CDRs have favored canonical structures with the exception of the CDR-H3, which comprises amino acid sequences that may be highly variable in sequence and length between antibodies resulting in a variety of three-dimensional structures in antigen-binding domains (Nikoloudis, D. et al., 2014. PeerJ. 2:e456). In some cases, CDR-H3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia PA. 2012. Ch. 3, p 47-54, the contents of which are herein incorporated by reference in their entirety).

As used herein, the term “Fv” refers to an antibody fragment comprising the minimum fragment on an antibody needed to form a complete antigen-binding site. These regions consist of a dimer of one heavy chain and one light chain variable domain in tight, non-covalent association. Fv fragments can be generated by proteolytic cleavage, but are largely unstable. Recombinant methods are known in the art for generating stable Fv fragments, typically through insertion of a flexible linker between the light chain variable domain and the heavy chain variable domain [to form a single chain Fv (scFv)] or through the introduction of a disulfide bridge between heavy and light chain variable domains (Strohl, W. R. Therapeutic Antibody Engineering. Woodhead Publishing, Philadelphia PA. 2012. Ch. 3, p 46-47, the contents of which are herein incorporated by reference in their entirety).

Antibody “light chains” from any vertebrate species can be assigned to one of two clearly distinct types, called kappa and lambda based on amino acid sequences of their constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2a, IgG2b, IgG2c, IgG3, IgG4, IgA, and IgA2.

As used herein, the term “single-chain Fv” or “scFv” as used herein, refers to a fusion protein of V_{H} and V_{L} antibody domains, wherein these domains are linked together into a single polypeptide chain. In some embodiments, the Fv polypeptide linker enables the scFv to form the desired structure for antigen binding.

The term “diabodies” refers to small antibody fragments with two antigen-binding sites, which fragments comprise a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993), the contents of each of which are incorporated herein by reference in their entirety.

The term “intrabody” refers to a form of antibody that is not secreted from a cell in which it is produced, but instead target one or more intracellular protein. Intrabodies may be used to affect a multitude of cellular processes including, but not limited to intracellular trafficking, transcription, translation, metabolic processes, proliferative signaling and cell division. In some embodiments, methods of the present invention may include intrabody-based therapies. In some such embodiments, variable domain sequences and/or CDR sequences disclosed herein may be incorporated into one or more construct for intrabody-based therapy. In some cases, intrabodies of the invention may target one or more glycated intracellular protein or may modulate the interaction between one or more glycated intracellular protein and an alternative protein.

The term “chimeric antigen receptor” or “CAR” as used herein, refers to artificial receptors that are engineered to be expressed on the surface of immune effector cells resulting in specific targeting of such immune effector cells to cells expressing entities that bind with high affinity to the artificial receptors. CARs may be designed to include one or more segments of an antibody, antibody variable domain and/or antibody CDR, such that when such CARs are expressed on immune effector cells, the immune effector cells bind and clear any cells that are recognized by the antibody portions of the CARs. In some cases, CARs are designed to specifically bind cancer cells, leading to immune-regulated clearance of the cancer cells.

The term “monoclonal antibody” as used herein refers to an antibody obtained from a population of substantially homogeneous cells (or clones), i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

The term “bispecifc antibody” as used herein refers to an antibody or antibody fragment capable of binding to two targets of different structure, such as two different antigens or two different epitopes on the same antigen.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and capacity.

In some embodiments, antibodies of the present invention may be antibody mimetics. The term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affilins, affitins, anticalins, avimers, DARPins, Fynomers and Kunitz and domain peptides. In other embodiments, antibody mimetics may include one or more non-peptide region.

As used herein, the term “antibody variant” refers to a biomolecule resembling an antibody in structure and/or function comprising some differences in their amino acid sequence, composition or structure as compared to a native antibody.

Antibodies of the present invention may be developed through immunizing a host with a particular antigen. As used herein, an “antigen” is an entity which induces or evokes an immune response in an organism. An immune response is characterized by the reaction of the cells, tissues and/or organs of an organism to the presence of a foreign entity. Such an immune response typically leads to the production by the organism of one or more antibodies against the foreign entity, e.g., antigen or a portion of the antigen. In some cases, methods of immunization may be altered based on one or more desired immunization outcomes. As used herein, the term “immunization outcome” refers to one or more desired effects of immunization. Examples include high antibody titers and/or increased antibody specificity for a target of interest.

The affinity between an antibody and a target or ligand (such as an antigen used to generate a given antibody) may be measured in terms of K_{D} using one or more binding assays as described herein. Depending on the desired application for a given antibody, varying KD values may be desirable. High affinity antibodies typically form ligand bonds with a KD of about 10⁻⁵ M or less, e.g. about 10⁻⁶ M or less, about 10⁻⁷ M or less, about 10⁻⁸ M or less, about 10⁻⁹ M or less, about 10⁻¹⁰ M or less, about 10⁻¹¹ M or less or about 10⁻¹² M or less.

### Recombinant Antibodies

Recombinant antibodies of the invention may be generated using standard techniques known in the art. In some embodiments, recombinant antibodies may be anti-glycan antibodies. Further antibodies may be anti-STn antibodies (e.g. anti-GcSTn or anti-AcSTn antibodies). Recombinant antibodies of the invention may be produced using variable domains obtained from hybridoma cell-derived antibodies produced according to methods described herein. Heavy and light chain variable region cDNA sequences of antibodies may be determined using standard biochemical techniques. Total RNA may be extracted from antibody-producing hybridoma cells and converted to cDNA by reverse transcriptase (RT) polymerase chain reaction (PCR). PCR amplification may be carried out on resulting cDNA to amplify variable region genes. Such amplification may comprise the use of primers specific for amplification of heavy and light chain sequences. In other embodiments, recombinant antibodies may be produced using variable domains obtained from other sources. This includes the use of variable domains selected from one or more antibody fragment library, such as an scFv library used in antigen panning. Resulting PCR products may then be subcloned into plasmids for sequence analysis. Once sequenced, antibody coding sequences may be placed into expression vectors. For humanization, coding sequences for human heavy and light chain constant domains may be used to substitute for homologous murine sequences. The resulting constructs may then be transfected into mammalian cells for large scale translation.

### Anti-Tn Antibodies

In some embodiments, recombinant antibodies of the invention may be anti-Tn antibodies. Such antibodies may bind to targets comprising Tn. Anti-Tn antibodies may be specific for Tn or may bind other modified forms of Tn, such as Tn linked to other moieties, including, but not limited to additional carbohydrate residues. In some cases anti-Tn antibodies may be anti-sialyl-Tn antibodies. Such antibodies may bind to targets comprising sialylated Tn comprising Neu5Ac and/or targets comprising sialylated Tn comprising Neu5Gc. Some anti-Tn antibodies may bind specifically to clusters of Tn antigen.

### Anti-STn Antibodies

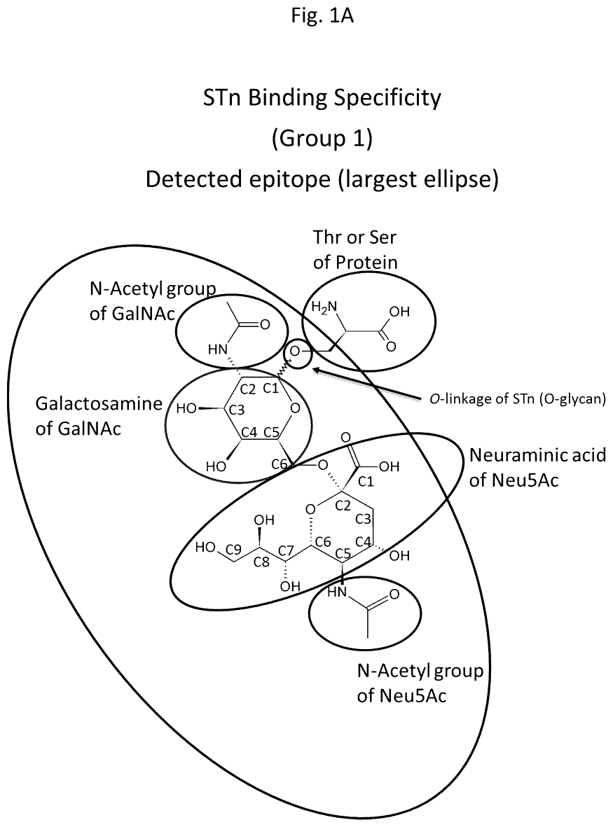

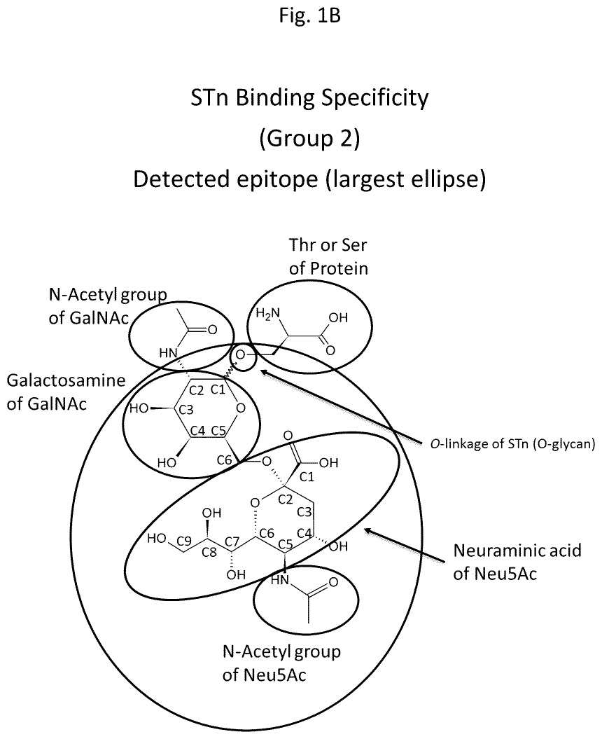

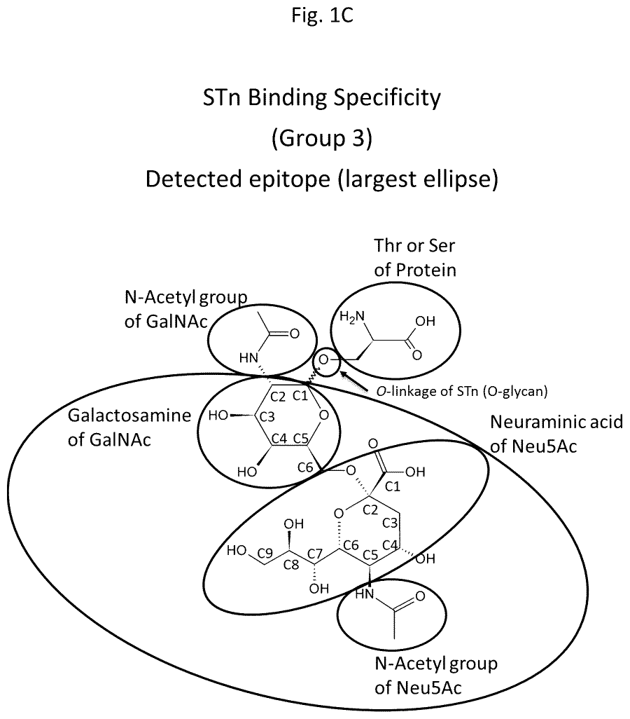

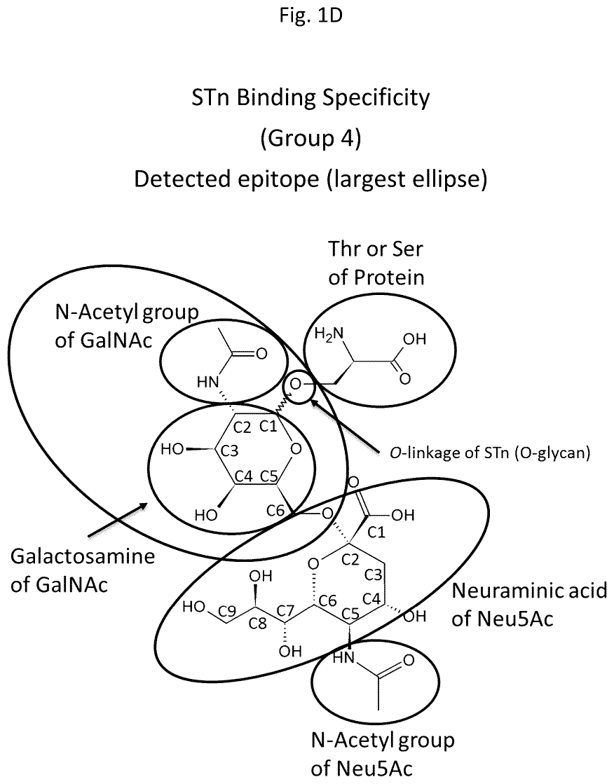

In some embodiments, antibodies of the invention may specifically bind to antigens comprising STn. Anti-STn antibodies of the invention may be categorized by their binding to specific portions of STn antigens and/or by their specificity for AcSTn versus GcSTn. In some cases, anti-STn antibodies of the invention are Group 1 antibodies. “Group 1” antibodies according to the invention are antibodies capable of binding AcSTn and GcSTn. Such antibodies may also be referred to herein as pan-STn antibodies due to their ability to associate with a wider range of STn structures. In some embodiments, Group 1 antibodies may associate with the portion of STn indicated by the large oval in FIG. 1A. In some cases, anti-STn antibodies of the invention are Group 2 antibodies. “Group 2” antibodies, according to the invention, are antibodies capable of binding STn as well as some related structures that include an O-linkage to serine or threonine. In some embodiments, Group 2 antibodies may associate with glycans comprising a sialylated galactose residue. In some cases, Group 2 antibodies may associate with the portion of STn indicated by the large oval in FIG. 1B. Some Group 2 antibodies preferably bind to structures with AcSTn over structures with GcSTn. Further anti-STn antibodies may be Group 3 antibodies. As referred to herein, “Group 3” antibodies are antibodies capable of binding STn, but may also bind a broader set of related structures. Unlike Group 2 antibodies, Group 3 antibodies do not require that such structures have an O-linkage to serine or threonine. In some embodiments, Group 3 antibodies may associate with the portion of STn indicated by the large oval in FIG. 1C. Finally, some anti-STn antibodies of the invention may be Group 4 antibodies. As referred to herein, “Group 4” antibodies are capable of binding to both AcSTn and GcSTn as well as the un-sialylated Tn antigen, and therefore have broader specificity. In some embodiments, Group 4 antibodies may associate with the portion of STn indicated by the large oval in FIG. 1D.

In some cases, anti-STn antibodies of the invention may bind specifically to clusters of STn on a particular antigen or cell surface. Some such antibodies may recognize epitopes formed by the clustering of STn, including epitopes that include areas of contact between neighboring STn structures. Such epitopes may be formed by the clustering of 2, 3, 4, 5, 6, 7, 8, 9, 10 or more STn structures.

### IgG Synthesis

IgG antibodies (e.g. IgG1, IgG2, IgG3 or IgG4) comprising one or more variable domain and/or CDR amino acid sequences presented herein (or fragment or variants thereof) may be synthesized for further testing and/or product development. Such antibodies may be produced by insertion of one or more segments of cDNA encoding desired amino acid sequences into expression vectors suited for IgG production. Expression vectors may comprise mammalian expression vectors suitable for IgG expression in mammalian cells. Mammalian expression of IgGs may be carried out to ensure that antibodies produced comprise modifications (e.g. glycosylation) characteristic of mammalian proteins and/or to ensure that antibody preparations lack endotoxin and/or other contaminants that may be present in protein preparations from bacterial expression systems.

### Antigen Selection

Methods of the present invention, including glycoprofiling methods, may be used to identify and/or select therapeutic target antigens. As used herein, the term “therapeutic target antigen” refers to an antigen for which development of one or more antibodies that specifically recognize such antigens would be desired for treatment of one or more diseases, disorders and/or conditions. According to such methods, data may be obtained and/or analyzed to determine the overall abundance of therapeutic target antigens in target tissues and/or the overall abundance in non-target tissues. Antigen potential as a therapeutic target antigen may be determined by assessing antigen abundance in target tissue and comparing to abundance in non-target tissue. In some cases, antigen potential as a target may be determined by comparing antigen abundance among target tissues alone.

Therapeutic target antigens of the invention may comprise glycans and/or glycoconjugates (including, but not limited to glycoproteins, glycolipids, glycated peptides, polypeptides, etc.). In some embodiments, antigens may comprise sialylated glycans. Mucins are a family of proteins with heavy glycosylation. Mucin-associated glycans may comprise high levels of sialoglycans, depending on the source. They are abundant in submaxillary glands and excreted in saliva and mucous. Sialoglycans found in mucins include α2,6-sialylated N-acetylgalactosamine (STn).

Animal-derived submaxillary mucins may be used as antigens to generate anti-STn antibodies in immunogenic hosts. Submaxillary mucins from different species differ in their STn content with regard to form of sialic acid [Neu5Ac-STn (AcSTn) versus Neu5GcSTn (GcSTn) versus KDN-STn forms.] Porcine submaxillary mucin (PSM) is especially rich in GcSTn, which represents about 90% of total STn. STn from bovine submaxillary mucin (BSM) has nearly equal percentages of GcSTn and AcSTn. Ovine submaxillary mucin (OSM) is especially rich in AcSTn, where it makes up about 90% of the total STn. PSM has high levels of Neu5Gc-containing mucin-type, glycoproteins. Among sources currently known to be high in Neu5Gc content is red meat. Neu5Gc content is especially high in the submaxillary glands of organisms expressing the cytidine monophosphate-N-acetylneuraminic acid hydroxylase (CMAH) gene. In such organisms, the submaxillary gland is an especially rich source of Neu5Gc due to the high expression of the CMAH enzyme, which catalyzes the reaction to produce the Neu5Gc precursor, CMP-Neu5Ac (Chandrasekharan, K. et al., 2010. Sci Transl Med. 2(42): 42ra54).

In some cases, PSM may be used to prevent a pan-anti-Neu5Gc response and induce a more specific immune response against GcSTn. OSM may be used in immunizations to generate antibodies in immunogenic hosts that are more likely to be specific for AcSTn. In some embodiments, PSM may be used to develop an antibody that is GcSTn-specific. Such antibodies may have little cross-reactivity with Neu5Ac-STn or Tn. In some cases, such antibodies may bind GcSTn, with reduced affinity for AcSTn.

In some embodiments, antigens may be subjected to enzymatic digestion prior to immunization to modulate the resulting immune response in immunogenic hosts. In one example, submaxillary mucins may be treated with trypsin or proteinase K enzymes prior to immunization. The activity of such enzymes may help to cleave off and thereby reduce the percentage and variability of non-STn epitopes. Glycan moieties may shield regions of the peptide where they are attached from enzymatic proteolysis and thereby remain intact.

Antibody titers resulting from immunizations may comprise different levels depending on the type and amount of antigen used in such immunizations. In some cases, certain antigens may be selected for use in immunizations based on the expected titer.

As used herein, an “adjuvant” is a pharmacological or immunological agent that modifies the effect of other agents. Adjuvants may include, but are not limited chemical compositions, biomolecules, therapeutics, and/or therapeutic regimens. Adjuvants may include Freund's adjuvant (complete and/or incomplete), immunostimulatory oligonucleotides [e.g. CpG oligodeoxynucleotides (ODNs,] mineral-containing compositions, bacterial ADP-ribosylating toxins, bioadhesives, mucoadhesives, microparticles, lipids, liposomes, muramyl peptides, N-oxidized polyethylene-piperazine derivatives, saponins and/or immune stimulating complexes (ISCOs). In some embodiments, adjuvants may comprise oil-in-water emulsions (e.g. sub-micron oil-in-water emulsions). Further useful adjuvants may include any of those disclosed in International Publication No. WO2013151649, US Patent Publication No. US20120027813 or US2014/0178365 and/or U.S. Pat. No. 8,506,966, the contents of each of which are herein incorporated by reference in their entirety.

### Polyclonal and Monoclonal Antibody Production

Antibodies developed according to the methods described herein may be polyclonal or monoclonal or recombinant, produced by methods known in the art or as described herein. Antibodies may be labeled for purposes of detection with a detectable label known by one of skill in the art. The label can be a radioisotope, fluorescent compound, chemiluminescent compound, enzyme, or enzyme co-factor, or any other labels known in the art. Further antibodies may be multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), intracellularly made antibodies (i.e., intrabodies), and epitope-binding fragments of any of the above. Antibodies of the present invention can be from any animal origin including birds and mammals. Such antibodies may be of (but are not limited to) human, murine (e.g., mouse and rat), donkey, sheep, rabbit, goat, guinea pig, camel, horse, or chicken origin. The antibodies of the present invention can be monospecific or multispecific (e.g., bispecific, trispecific, or of greater multispecificity). Multispecific antibodies can be specific for different epitopes of a target antigen of the present invention, or can be specific for both a target antigen of the present invention, and a heterologous epitope, such as a heterologous glycan, peptide or solid support material. (See, e.g., WO 93/17715; WO 92/08802; WO 91/00360; WO 92/05793; Tutt, A. et al., Trispecific F(ab)3 derivatives that use cooperative signaling via the TCR/CD3 complex and CD2 to activate and redirect resting cytotoxic T cells. J Immunol. 1991 Jul. 1; 147(1):60-9; U.S. Pat. Nos. 4,474,893; 4,714,681; 4,925,648; 5,573,920; 5,601,819; and Kostelny, S. A. et al., Formation of a bispecific antibody by the use of leucine zippers. J Immunol. 1992 Mar. 1; 148(5):1547-53), the contents of each of which are herein incorporated by reference in their entirety.

Anti-glycan antibodies of the present invention comprising monoclonal antibodies may be prepared using well-established methods known by those skilled in the art. In one embodiment, the monoclonal antibodies are prepared using hybridoma technology (Kohler, G. et al., Continuous cultures of fused cells secreting antibody of predefined specificity. Nature. 1975 Aug. 7; 256(5517):495-7). For hybridoma formations, first, a mouse, hamster, or other appropriate host animal, is typically immunized with an immunizing agent (e.g., a target antigen of the invention) to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell (Goding, J. W., Monoclonal Antibodies: Principles and Practice. Academic Press. 1986; 59-1031). Immortalized cell lines are usually transformed mammalian cells, particularly myeloma cells of rodent, rabbit, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif and the American Type Culture Collection, Manassas, Va. Human myeloma and mouse-human heteromyeloma cell lines also have been described for the production of human monoclonal antibodies (Kozbor, D. et al., A human hybrid myeloma for production of human monoclonal antibodies. J Immunol. 1984 December; 133(6):3001-5; Brodeur, B. et al., Monoclonal Antibody Production Techniques and Applications. Marcel Dekker, Inc., New York. 1987; 33:51-63).

In some embodiments, myeloma cells may be subjected to genetic manipulation. Such manipulation may be carried out using zinc-finger nuclease (ZFN) mutagenesis as described herein. Alternatively, transfection methods known in the art may be used. NS0 myeloma cells or other mouse myeloma cell lines may be used. For example, Sp2/0-Ag14 can be an alternative cell line for hybridoma development. Transcription Activator-Like Effector Nucleases (TALENs)-induced gene editing provides an alternative gene knock out method. TALENs are artificial restriction enzymes generated by fusing the TAL effector DNA binding domain to a DNA cleavage domain. Similar to ZFNs, TALENs induce double-strand breaks at desired loci that can be repaired by error-prone NHEJ to yield insertions/deletions at the break sites (Wood, A. J. et al., Targeted genome editing across species using ZFNs and TALENs. Science. 2011 Jul. 15; 333(6040):307). Cellectis Bioresearch (Cambridge, MA) provides the service of TALEN design and plasmid construction.

The culture medium in which the hybridoma cells are cultured can then be assayed for the presence of monoclonal antibodies. Preferably, the binding specificity (i.e., specific immunoreactivity) of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known by those skilled in the art. The binding specificity of the monoclonal antibody can, for example, be determined by Scatchard analysis (Munson, P. J. et al., Ligand: a versatile computerized approach for characterization of ligand-binding systems. Anal Biochem. 1980 Sep. 1; 107(1):220-39). In some cases, antibody specificity for regions of a given antigen may be characterized by chemically modifying the antigens prior to assaying for antibody binding. In one example, periodate treatment may be used to destroy the C6 side chain of sialic acids. Assays may be conducted with and without periodate treatment to reveal whether or not binding in untreated samples is sialic acid-specific. In some cases, antigens comprising 9-O-acetylated sialic acid may be subjected to mild base treatment (e.g. with 0.1 M NaOH) to destroy 9-O-acetyl groups. Assays may be conducted with and without mild base treatment to reveal whether or not binding in untreated samples depends on 9-O-acetylation of sialic acid.

After the desired hybridoma cells are identified, the clones may be subcloned by limiting dilution procedures and grown by standard methods. Suitable culture media for this purpose include, for example, Dulbecco's Modified Eagle's Medium or RPMI-1640 medium. Alternatively, the hybridoma cells may be grown in vivo as ascites in a mammal.

Alternative methods to clone hybridomas may include those provided by kits from STEMCELL Technologies (Vancouver, BC, Canada), e.g. CLONACELL™-HY kit, containing methylcellulose-based semi-solid medium and other media and reagents, to support the selection and growth of hybridoma clones. However, the media in this kit contain FCS, which provides an exogenous source for Neu5Gc incorporation. Though the machinery for endogenous Neu5Gc synthesis is destroyed in Cmah^{-/-} hybridoma, Neu5Gc incorporated from the culture media may also pose a problem in some cases (Bardor, M. et al., Mechanism of uptake and incorporation of the non-human sialic acid N-glycolylneuraminic acid into human cells. J Biol Chem. 2005. 280: 4228-4237). In such instances, the culture media may be supplemented with Neu5Ac to eliminate Neu5Gc incorporation by metabolic competition (Ghaderi, D. et al., Implications of the presence of N-glycolylneuraminic acid in recombinant therapeutic glycoproteins. Nat Biotechnol. 2010. 28: 863-867).

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In another embodiment, the monoclonal antibodies of the present invention can also be made by recombinant DNA methods, such as those described in U.S. Pat. No. 4,816,567, which is hereby incorporated by reference in its entirety. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

In some embodiments, antibodies of the present invention may be produced by various procedures known by those skilled in the art. For the production of polyclonal antibodies in vivo, host animals, such as rabbits, rats, mice, cows, horses, donkeys, chickens, monkeys, sheep or goats, are immunized with either free or carrier-coupled antigens, for example, by intraperitoneal and/or intradermal injection. In some embodiments, injection material may be an emulsion containing about 100 μg of antigen or carrier protein. In some embodiments, injection materials comprise a glycan-rich composition such as non-human mammalian submaxillary mucin in solution. Various adjuvants can also be used to increase the immunological response, depending on the host species. Adjuvants include, but are not limited to, Freund's (complete and incomplete), mineral gels such as aluminum hydroxide, surface active substances such as lysolecithin, pluronic polyols, polyanions, peptides, oil emulsions, TITERMAX® (CytRx Corp, Los Angeles, CA), keyhole limpet hemocyanins, dinitrophenol, and potentially useful human adjuvants such as BCG (bacille Calmette-Guerin) and Corynebacterium parvum. Such adjuvants are also well known in the art. Several booster injections may be needed, for instance, at intervals of about two weeks, to provide a useful titer of antibody which can be detected, for example, by ELISA assay using glycans and/or free peptide adsorbed to a solid surface. The titer of antibodies in serum from an immunized animal can be increased by selection of antibodies, e.g., by adsorption of antigens onto a solid support and elution of the selected antibodies according to methods well known in the art.

Anti-glycan antibodies, variants and fragments thereof may be selected and produced using high throughput methods of discovery. In one embodiment, anti-glycan antibodies comprising synthetic antibodies, variants or fragments thereof are produced through the use of display libraries. The term “display” as used herein, refers to the expression or “display” of proteins or peptides on the surface of a given host. The term “library” as used herein, refers to a collection of unique cDNA sequences and/or the proteins that are encoded by them. A library may contain from as little as two unique cDNAs to hundreds of billions of unique cDNAs. In some embodiments, anti-glycan antibodies comprising synthetic antibodies are produced using antibody display libraries or antibody fragment display libraries. The term “antibody fragment display library” as used herein, refers to a display library wherein each member encodes an antibody fragment containing at least one variable region of an antibody. Such antibody fragments are preferably Fab fragments, but other antibody fragments such as single-chain variable fragments (scFvs) are contemplated as well. In an Fab antibody fragment library, each Fab encoded may be identical except for the amino acid sequence contained within the variable loops of the complementarity determining regions (CDRs) of the Fab fragment. In an alternative or additional embodiment, amino acid sequences within the individual V_{H} and/or V_{L} regions may differ as well.

Display libraries may be expressed in a number of possible hosts including, but not limited to yeast, bacteriophage, bacteria and retroviruses. Additional display technologies that may be used include ribosome-display, microbead-display and protein-DNA linkage techniques. In a preferred embodiment, Fab display libraries are expressed in yeast or in bacteriophages (also referred to herein as “phages” or “phage particles”. When expressed, the Fabs decorate the surface of the phage or yeast where they can interact with a given antigen. An antigen comprising a glycan or other antigen from a desired target may be used to select phage particles or yeast cells expressing antibody fragments with the highest affinity for that antigen. The DNA sequence encoding the CDR of the bound antibody fragment can then be determined through sequencing using the bound particle or cell. In one embodiment, positive selection is used in the development of antibodies. In some embodiments, negative selection is utilized in the development of antibodies. In some embodiments, both positive and negative selection methods are utilized during multiple rounds of selection in the development of antibodies using display libraries.

In yeast display, cDNA encoding different antibody fragments are introduced into yeast cells where they are expressed and the antibody fragments are “displayed” on the cell surface as described by Chao et al. (Chao, G. et al., Isolating and engineering human antibodies using yeast surface display. Nat Protoc. 2006; 1(2):755-68). In yeast surface display, expressed antibody fragments contain an additional domain comprising the yeast agglutinin protein, Aga2p. This domain allows the antibody fragment fusion protein to attach to the outer surface of the yeast cell through the formation of disulphide bonds with surface-expressed Aga1p. The result is a yeast cell, coated in a particular antibody fragment. Display libraries of cDNA encoding these antibody fragments are utilized initially in which the antibody fragments each have a unique sequence. These fusion proteins are expressed on the cell surface of millions of yeast cells where they can interact with a desired antigenic target antigen, incubated with the cells. Target antigens may be covalently or otherwise modified with a chemical or magnetic group to allow for efficient cell sorting after successful binding with a suitable antibody fragment takes place. Recovery may be by way of magnetic-activated cell sorting (MACS), fluorescence-activated cell sorting (FACS) or other cell sorting methods known in the art. Once a subpopulation of yeast cells is selected, the corresponding plasmids may be analyzed to determine the CDR sequence.

Bacteriophage display technology typically utilizes filamentous phage including, but not limited to fd, F1 and M13 virions. Such strains are non-lytic, allowing for continued propagation of the host and increased viral titres. Examples of phage display methods that can be used to make the antibodies of the present invention include those disclosed in Miersch et al. (Miersch, S. et al., Synthetic antibodies: Concepts, potential and practical considerations. Methods. 2012 August; 57(4):486-98), Bradbury et al. (Bradbury, A. R. et al., Beyond natural antibodies: the power of in vitro display technologies. Nat Biotechnol. 2011 March; 29(3):245-54), Brinkman et al. (Brinkmann, U. et al., Phage display of disulfide-stabilized Fv fragments. J Immunol Methods. 1995 May 11; 182(1):41-50); Ames et al. (Ames, R. S. et al., Conversion of murine Fabs isolated from a combinatorial phage display library to full length immunoglobulins. J Immunol Methods. 1995 Aug. 18; 184(2):177-86); Kettleborough et al. (Kettleborough, C. A. et al., Isolation of tumor cell-specific single-chain Fv from immunized mice using phage-antibody libraries and the re-construction of whole antibodies from these antibody fragments. Eur J Immunol. 1994 April; 24(4):952-8); Persic et al. (Persic, L. et al., An integrated vector system for the eukaryotic expression of antibodies or their fragments after selection from phage display libraries. Gene. 1997 Mar. 10; 187(1):9-18).; PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5, 969,108, each of which is incorporated herein by reference in its entirety. Antibody fragment expression on bacteriophages may be carried out by inserting the cDNA encoding the fragment into the gene expressing a viral coat protein. The viral coat of filamentous bacteriophages is made up of five coat proteins, encoded by a single-stranded genome. Coat protein pIII is the preferred protein for antibody fragment expression, typically at the N-terminus. If antibody fragment expression compromises the function of pIII, viral function may be restored through coexpression of a wild type pIII, although such expression will reduce the number of antibody fragments expressed on the viral coat, but may enhance access to the antibody fragment by the target antigen. Expression of viral as well as antibody fragment proteins may alternatively be encoded on multiple plasmids. This method may be used to reduce the overall size of infective plasmids and enhance the transformation efficiency.

As described above, after selection of a host expressing a high affinity antibody or antibody fragment, the coding regions from the antibody or antibody fragment can be isolated and used to generate whole antibodies, including human antibodies, or any other desired antigen binding fragment, and expressed in any desired host, including mammalian cells, insect cells, plant cells, yeast, and bacteria, e.g., as described in detail below.

The DNA sequence encoding a high affinity antibody can be mutated for additional rounds of selection in a process known as affinity maturation. The term “affinity maturation”, as used herein, refers to a method whereby antibodies are produced with increasing affinity for a given antigen through successive rounds of mutation and selection of antibody- or antibody fragment-encoding cDNA sequences. In a preferred embodiment, this process is carried out in vitro. To accomplish this, amplification of CDR coding sequences may be carried out using error-prone PCR to produce millions of copies containing mutations including, but not limited to point mutations, regional mutations, insertional mutations and deletional mutations. As used herein, the term “point mutation” refers to a nucleic acid mutation in which one nucleotide within a nucleotide sequence is changed to a different nucleotide. As used herein, the term “regional mutation” refers to a nucleic acid mutation in which two or more consecutive nucleotides are changed to different nucleotides. As used herein, the term “insertional mutation” refers to a nucleic acid mutation in which one or more nucleotides are inserted into a nucleotide sequence. As used herein, the term “deletional mutation” refers to a nucleic acid mutation in which one or more nucleotides are removed from a nucleotide sequence. Insertional or deletional mutations may include the complete replacement of an entire codon or the change of one codon to another by altering one or two nucleotides of the starting codon.

Mutagenesis may be carried out on CDR-encoding cDNA sequences to create millions of mutants with singular mutations in CDR heavy and light chain regions. In another approach, random mutations are introduced only at CDR residues most likely to improve affinity. These newly generated mutagenic libraries can be used to repeat the process to screen for clones that encode antibody fragments with even higher affinity for the target antigen. Continued rounds of mutation and selection promote the synthesis of clones with greater and greater affinity (Chao, G. et al., Isolating and engineering human antibodies using yeast surface display. Nat Protoc. 2006; 1(2):755-68).

Examples of techniques that can be used to produce antibodies and antibody fragments, such as Fabs and scFvs, include those described in U.S. Pat. Nos. 4,946,778 and 5,258,498; Miersch et al. (Miersch, S. et al., Synthetic antibodies: Concepts, potential and practical considerations. Methods. 2012 August; 57(4):486-98), Chao et al. (Chao, G. et al., Isolating and engineering human antibodies using yeast surface display. Nat Protoc. 2006; 1(2):755-68), Huston et al. (Huston, J. S. et al., Protein engineering of single-chain Fv analogs and fusion proteins. Methods Enzymol. 1991; 203:46-88); Shu et al. (Shu, L. et al., Secretion of a single-gene-encoded immunoglobulinfrom myeloma cells. Proc Natl Acad Sci USA. 1993 Sep. 1; 90(17):7995-9); and Skerra et al. (Skerra, A. et al., Assembly of a functional immunoglobulin Fv fragment in Escherichia coli. Science. 1988 May 20; 240(4855):1038-41), each of which is incorporated herein by reference in its entirety.

For some uses, including the in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal immunoglobulin and a human immunoglobulin constant region. Methods for producing chimeric antibodies are known in the art. (Morrison, S. L., Transfectomas provide novel chimeric antibodies. Science. 1985 Sep. 20; 229(4719):1202-7; Gillies, S. D. et al., High-level expression of chimeric antibodies using adapted cDNA variable region cassettes. J Immunol Methods. 1989 Dec. 20; 125(1-2):191-202.; and U.S. Pat. Nos. 5,807,715; 4,816,567; and 4,816,397, which are incorporated herein by reference in their entirety). Humanized antibodies are antibody molecules from non-human species that bind to the desired antigen and have one or more complementarity determining regions (CDRs) from the nonhuman species and framework regions from a human immunoglobulin molecule. Often, framework residues in the human framework regions are substituted with corresponding residues from the CDR and framework regions of the donor antibody to alter, preferably improve, antigen binding. These framework substitutions are identified by methods well known in the art, e.g., by modeling of the interactions of the CDR and framework residues to identify framework residues important for antigen binding, and by sequence comparison to identify unusual framework residues at particular positions. (U.S. Pat. Nos. 5,693,762 and 5,585,089; Riechmann, L. et al., Reshaping human antibodies for therapy. Nature. 1988 Mar. 24; 332(6162):323-7, which are incorporated herein by reference in their entireties). Antibodies can be humanized using a variety of techniques known in the art, including, for example, CDR-grafting (EP 239,400; PCT publication WO 91/09967; U.S. Pat. Nos. 5,225,539; 5,530,101; and 5,585,089); veneering or resurfacing (EP 592,106; EP 519,596; Padlan, E. A., A possible procedure for reducing the immunogenicity of antibody variable domains while preserving their ligand-binding properties. Mol Immunol. 1991 April-May; 28(4-5):489-98; Studnicka, G. M. et al., Human-engineered monoclonal antibodies retain full specific binding activity by preserving non-CDR complementarity-modulating residues. Protein Eng. 1994 June; 7(6):805-14; Roguska, M. A. et al., Humanization of murine monoclonal antibodies through variable domain resurfacing. Proc Natl Acad Sci USA. 1994 Feb. 1; 91(3):969-73); and chain shuffling (U.S. Pat. No. 5,565,332); each of which is incorporated herein by reference in their entirety. Humanized antibodies of the present invention may be developed for desired binding specificity, complement-dependent cytotoxicity, and antibody-dependent cellular-mediated cytotoxicity, etc.

Completely human antibodies are particularly desirable for therapeutic treatment of human patients, so as to avoid or alleviate immune reaction to foreign protein. Human antibodies can be made by a variety of methods known in the art, including the antibody display methods described above, using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin polynucleotides. For example, the human heavy and light chain immunoglobulin polynucleotide complexes can be introduced randomly, or by homologous recombination, into mouse embryonic stem cells. Alternatively, the human variable region, constant region, and diversity region may be introduced into mouse embryonic stem cells, in addition to the human heavy and light chain polynucleotides. The mouse heavy and light chain immunoglobulin polynucleotides can be rendered nonfunctional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a glycan, glycoconjugate and/or polypeptide of the invention.

Thus, using such a technique, it is possible to produce useful human IgG, IgA, IgM, IgD and IgE antibodies. For an overview of the technology for producing human antibodies, see Lonberg and Huszar (Lonberg, N. et al., Human antibodies from transgenic mice. Int Rev Immunol. 1995; 13(1):65-93). For a detailed discussion of the technology for producing human antibodies and human monoclonal antibodies and protocols for producing such antibodies, see, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; 5,939,598; 6,075,181; and 6,114,598, each of which are incorporated by reference herein in their entirety. In addition, companies such as Abgenix, Inc. (Fremont, Calif.), Protein Design Labs, Inc. (Mountain View, Calif.) and Genpharm (San Jose, Calif.) can be engaged to provide human antibodies directed against a selected antigen using technology similar to the above described technologies.

Once an antibody molecule of the present invention has been produced by an animal, a cell line, chemically synthesized, or recombinantly expressed, it can be purified (i.e., isolated) by any method known in the art for the purification of an immunoglobulin or polypeptide molecule, for example, by chromatography (e.g., ion exchange, affinity, particularly by affinity for the specific antigen, Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The preparation of antibodies, whether monoclonal or polyclonal, is known in the art. Techniques for the production of antibodies are well known in the art and described, e.g. in Strohl, W. R. Therapeutic Antibody Engineering. Woodhead Publishing, Philadelphia PA. 2012.

### Immunogenic Hosts

In some embodiments, antibodies of the present invention may be developed through the use of non-human animals as hosts for immunization, referred to herein as “immunogenic hosts”. In some embodiments, immunogenic hosts are mammals. In some embodiments, immunogenic hosts are transgenic knockout mice. Antigens comprising target sites and/or epitope targets of for antibody production may be used to contact immunogenic hosts in order to stimulate an immune response and produce antibodies in the immunogenic host that specifically bind the target sites and/or epitope targets present on the antigens introduced.

According to some methods of the present invention, the development of antibodies may comprise immunizing mice that have had the Cmah gene disrupted. Such mutations may result in more human-like physiology in that Neu5Gc, the immunogenic, non-human form of sialic acid, is no longer produced in such mice. Other genes can be knocked out in the background of Cmah^{-/-} myeloma cells. For example, the alpha1,3-galactosyltransferase gene, which encodes an enzyme critical for the formation of an epitope highly-immunogenic to humans (Chung, C. H. et al., Cetuximab-induced anaphylaxis and IgE specific for galactose-alpha-1,3-galactose. N Engl J Med. 2008 Mar. 13; 358(11):1109-17), can be knocked out in the background of Cmah^{-/-} myeloma cells.

According to other methods of the present invention, wild type mice may be used for immunization. Such methods may sometimes be favorable for the production of antibodies that interact with AcSTn or pan-STn epitopes. In some cases, immune responses in wild type mice may be more robust.

Antibodies produced through immunization may be isolated from serum of the immunogenic hosts. Antibody producing cells from the immunogenic hosts may also be used to generate cell lines that produce the desired antibody. In some embodiments, screening for antibodies and/or antibody producing cells from the immunogenic host may be carried out through the use of enzyme-linked immunosorbent assays (ELISAs) and/or glycan arrays.

### Adjuvants

Immunization of immunogenic hosts with antigens described herein may comprise the use of one or more adjuvants. Adjuvants may be used to elicit a higher immune response in such immunogenic hosts. As such, adjuvants used according to the present invention may be selected based on their ability to affect antibody titers.

In some embodiments, water-in-oil emulsions may be useful as adjuvants. Water-in-oil emulsions may act by forming mobile antigen depots, facilitating slow antigen release and enhancing antigen presentation to immune components. Water-in-oil emulsion-based adjuvants include. Freund's adjuvant may be used as complete Freund's adjuvant (CFA,) which comprises mycobacterial particles that have been dried and inactivated, or incomplete Freund's adjuvant (IFA,) lacking such particles, may be used. Other water-in-oil-based adjuvants may include EMULSIGEN® (MVP Technologies, Omaha, NE). EMULSIGEN® comprises micron sized oil droplets that are free from animal-based components. It may be used alone or in combination with other adjuvants, including, but not limited to aluminum hydroxide and CARBIGEN™ (MVP Technologies, Omaha, NE).

In some embodiments, TITERMAX® adjuvant may be used. TITERMAX® is another water-in-oil emulsion comprising squalene as well as sorbitan monooleate 80 (as an emulsifier) and other components. In some cases, TITERMAX® may provide higher immune responses, but with decreased toxicity toward immunogenic hosts.

Immunostimmulatory oligonucleotides may also be used as adjuvants. Such adjuvants may include CpG oligodeoxynucleotide (ODN). CpG ODNs are recognized by Toll-like receptor 9 (TLR9) leading to strong immunostimulatory effects. Type C CpG ODNs induce strong IFN-α production from plasmacytoid dendritic cell (pDC) and B cell stimulation as well as IFN-γ production from T-helper (T_{H}) cells. CpG ODN adjuvant has been shown to significantly enhance pneumococcal polysaccharide (19F and type 6B)-specific IgG2a and IgG3 in mice. CpG ODN also enhanced antibody responses to the protein carrier CRM197, particularly CRM197-specific IgG2a and IgG3 (Chu et al., Infection Immunity 2000, vol 68(3):1450-6). Additionally, immunization of aged mice with pneumococcal capsular polysaccharide serotype 14 (PPS14) combined with a CpG-ODN restored IgG anti-PPS14 responses to young adult levels (Sen et al., Infection Immunity, 2006, 74(3):2177-86). CpG ODNs used according to the present invention may include class A, B or C ODNs. In some embodiments, ODNs may include any of those available commercially, such as ODN-1585, ODN-1668, ODN-1826, ODN-2006, ODN-2007, ODN-2216, ODN-2336, ODN-2395 and/or ODN-M362, each of which may be purchased, for example, from InvivoGen, (San Diego, CA). In some cases, ODN-2395 may be used. ODN-2395 is a class C CpG ODN that specifically stimulated human as well as mouse TLR9. These ODNs comprise phosphorothioate backbones and CpG palindromic motifs.

In some embodiments, immune stimulating complexes (ISCOMs) may be used as adjuvants. ISCOMs are spherical open cage-like structures (typically 40 nm in diameter) that are spontaneously formed when mixing together cholesterol, phospholipids and Quillaia saponins under a specific stoichiometry. ISCOM technology is proven for a huge variety of antigens from large glycoproteins such as gp340 from Epstein-Barr virus (a 340 kDa antigen consisting of 80% carbohydrates) down to carrier-conjugated synthetic peptides and small haptens such as biotin. Some ISCOMs are capable of generating a balanced immune response with both T_{H1} and T_{H2} characteristics. Immune response to ISCOMs is initiated in draining lymph nodes, but is efficiently relocated to the spleen, which makes it particularly suitable for generating monoclonal antibodies as well. In some embodiments, the ISCOM adjuvant AbISCO-100 (Isconova, Uppsala, Sweden) may be used. AbISCO-100 is a saponin-based adjuvant specifically developed for use in immunogenic hosts, such as mice, that may be sensitive to other saponins.

According to embodiments of the present invention, adjuvant components of immunization solutions may be varied in order to achieve desired results. Such results may include modulating the overall level of immune response and/or level of toxicity in immunogenic hosts.

### Antibody Fragment Display Library Screening Techniques

In some embodiments, antibodies of the present invention may be produced and/or optimized using high throughput methods of discovery. Such methods may include any of the display techniques (e.g. display library screening techniques) disclosed in International Patent Application No. WO2014074532, the contents of which are herein incorporated by reference in their entirety. In some embodiments, synthetic antibodies may be designed, selected or optimized by screening target antigens using display technologies (e.g. phage display technologies). Phage display libraries may comprise millions to billions of phage particles, each expressing unique antibody fragments on their viral coats. Such libraries may provide richly diverse resources that may be used to select potentially hundreds of antibody fragments with diverse levels of affinity for one or more antigens of interest (McCafferty, et al., 1990. Nature. 348:552-4; Edwards, B. M. et al., 2003. JMB. 334: 103-18; Schofield, D. et al., 2007. Genome Biol. 8, R254 and Pershad, K. et al., 2010. Protein Engineering Design and Selection. 23:279-88; the contents of each of which are herein incorporated by reference in their entirety). Often, the antibody fragments present in such libraries comprise scFv antibody fragments, comprising a fusion protein of V_{H} and V_{L} antibody domains joined by a flexible linker. In some cases, scFvs may contain the same sequence with the exception of unique sequences encoding variable loops of the complementarity determining regions (CDRs). In some cases, scFvs are expressed as fusion proteins, linked to viral coat proteins (e.g. the N-terminus of the viral pIII coat protein). V_{L} chains may be expressed separately for assembly with V_{H} chains in the periplasm prior to complex incorporation into viral coats.

Precipitated library members may be sequenced from the bound phage to obtain cDNA encoding desired scFvs. Such sequences may be directly incorporated into antibody sequences for recombinant antibody production, or mutated and utilized for further optimization through in vitro affinity maturation.

### Development of Cytotoxic Antibodies

In some embodiments, antibodies of the present invention may be capable of inducing antibody-dependent cell-mediated cytotoxicity (ADCC) and/or antibody-dependent cell phagocytosis (ADCP). ADCC is an immune mechanism whereby cells are lysed as a result of immune cell attack. Such immune cells may include CD56+ cells, CD3−natural killer (NK) cells, monocytes and neutrophils (Strohl, W. R. Therapeutic Antibody Engineering. Woodhead Publishing, Philadelphia PA. 2012. Ch. 8, p 186, the contents of which are herein incorporated by reference in their entirety).

In some cases, antibodies of the present invention may be engineered to comprise a given isotype depending on whether or not ADCC or ADCP is desired upon antibody binding. Such antibodies, for example, may be engineered according to any of the methods disclosed by Alderson, K. L. et al., (J Biomed Biotechnol. 2011. 2011:379123). In the case of mouse antibodies, different isotypes of antibodies are more effective at promoting ADCC. IgG2a, for example, is more effective at inducing ADCC than is IgG2b. Some antibodies of the present invention, comprising mouse IgG2b antibodies may be reengineered to comprise IgG2a antibodies. Such reengineered antibodies may be more effective at inducing ADCC upon binding cell-associated antigens.

In some embodiments, genes encoding variable regions of antibodies developed according to methods of the present invention may be cloned into mammalian expression vectors encoding human Fc regions. Such Fc regions may comprise Fc regions from human IgG1κ. IgG1κ Fc regions may comprise amino acid mutations known to enhance Fc-receptor binding and antibody-dependent cell-mediated cytotoxicity (ADCC).

In some embodiments, antibodies of the invention may be developed for antibody-drug conjugate (ADC) therapeutic applications. ADCs are antibodies in which one or more cargo (e.g. therapeutic compounds or cytotoxic agents) are attached (e.g. directly or via linker). ADCs are useful for delivery of such therapeutic compounds or cytotoxic agents to one or more target cells or tissues (Panowski, S. et al., 2014. mAbs 6:1, 34-45). In some cases, ADCs may be designed to bind to a surface antigen on a targeted cell. Upon binding, the entire antibody-antigen complex may be internalized and directed to a cellular lysosome. ADCs may then be degraded, releasing the bound cargo. Where the cargo is a cytotoxic agent, the target cell will be killed or otherwise disabled. Cytotoxic agents may include, but are not limited to cytoskeletal inhibitors (e.g. tubulin polymerization inhibitors such as maytansines or auristatins,) and DNA damaging agents (e.g. DNA polymerization inhibitors such as calcheamicins and duocarmycins).

In some embodiments, antibodies of the invention may be tested for their ability to promote cell death when developed as ADCs. Cell viability assays may be performed in the presence and absence of secondary antibody-drug conjugates. Antibodies with potent cell growth inhibition may then be used to design direct antibody-drug conjugates (ADCs). The use of such secondary antibody-drug conjugates in cell-based cytotoxic assays may allow for quick pre-screening of many ADC candidates. Based on such assays, an unconjugated antibody candidate is directly added to cells in the presence of a secondary antibody that is conjugated to one or more cytotoxic agents (referred to herein as a 2° ADC). Internalization of the antibody/2° ADC complex into cells that express a high density of the targeted antigen can achieve a dose-dependent drug release within the cells, causing a cytotoxic effect to kill the cells (e.g., tumor cells), while cells expressing a low density of the targeted antigen are not affected (e.g., normal cells).

ADCs of the invention may be designed to target cancer cells. Such ADCs may comprise antibodies directed to one or more tumor-associated carbohydrate antigen (TACA). In some cases, ADCs of the invention comprise anti-STn antibodies.

### Development of Chimeric Antigen Receptors

In some embodiments, methods of the invention may be used to develop a chimeric antigen receptor (CAR). CARs are transmembrane receptors expressed on immune cells that facilitate recognition and killing of target cells (e.g. tumor cells). Chimeric antigen receptors of the invention typically comprise three domains. These include an ectodomain, a transmembrane domain and an intracellular domain. Ectodomains facilitate binding to cellular antigens on target cells, while intracellular domains are typically involved in cell signaling functions to promote the killing of bound target cells. In some embodiments, CARS of the invention may have an extracellular domain with one or more antibody variable domains developed according to the methods described herein. CARs of the invention also include a transmembrane domain and cytoplasmic tail. Further structural features of CARs may include any of those disclosed in International Publication Nos. WO2012/079000 or WO2013/040557, the contents of each of which are herein incorporated by reference in their entirety.

In some embodiments, CARs of the invention may be engineered to target tumors. Such CARs may have specificity for one or more TACAs. In some case, ectodomains of these CARs may comprise one or more antibody variable domains developed according to the methods described herein. In some embodiments, CARs of the invention are expressed in T cells, referred to herein as “CAR-engineered T cells” or “CAR-Ts”. CAR-Ts may be engineered with CAR ectodomains having one or more antibody variable domains developed according to the methods of the present invention.

### Proteins and Variants

Antibodies and other proteins of the invention (e.g. antigens) of the present invention may exist as a whole polypeptide, a plurality of polypeptides or fragments of polypeptides, which independently may be encoded by one or more nucleic acids, a plurality of nucleic acids, fragments of nucleic acids, or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine. The amino acid sequences of antibodies of the invention may comprise naturally occurring amino acids and as such may be considered to be proteins, peptides, polypeptides, or fragments thereof. Alternatively, antibodies may comprise both naturally and non-naturally occurring amino acids.

The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70% homology to a native sequence, and preferably, they will be at least about 80%, more preferably at least about 90% homologous to a native sequence.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions or deletions of amino acid residues that still maintain the properties of the parent polypeptide.

The present invention contemplates several types of antibodies which are amino acid based including variants and derivatives. These include substitutional, insertional, deletion and covalent variants and derivatives. As such, included within the scope of this invention are antibody molecules containing substitutions, insertions and/or additions, deletions and covalently modifications. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In some embodiments, derivatives include native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which proteins of the invention are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337.

“Features” when referring to proteins are defined as distinct amino acid sequence-based components of a molecule. Features of the proteins of the present invention include surface manifestations, local conformational shape, folds, loops, half-loops, domains, half-domains, sites, termini or any combination thereof.

As used herein when referring to proteins the term “surface manifestation” refers to a polypeptide based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions.

As used herein when referring to proteins the term “half-domain” means portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid based embodiments is used synonymous with “amino acid residue” and “amino acid side chain”. A site represents a position within a peptide or polypeptide that may be modified, manipulated, altered, derivatized or varied within the polypeptide based molecules of the present invention.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH₂)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

### Isotopic Variations

Glycans, glycoproteins, antibodies and other products of the present invention may contain one or more atoms that are isotopes. As used herein, the term “isotope” refers to a chemical element that has one or more additional neutron. In one embodiment, compounds of the present invention may be deuterated. As used herein, the term “deuterated” refers to a substance that has had one or more hydrogen atoms replaced by deuterium isotopes. Deuterium isotopes are isotopes of hydrogen. The nucleus of hydrogen contains one proton while deuterium nuclei contain both a proton and a neutron. The glycans, glycoproteins, antibodies and other products of the present invention may be deuterated in order to change a physical property of the compound, such as stability, or to allow the compounds to be used in diagnostic and experimental applications.

### Conjugates and Combinations

It is contemplated by the present invention that the glycans, glycoproteins, antibodies and other products of the present invention may be complexed, conjugated or combined with one or more homologous or heterologous molecules. As used herein, “homologous molecule” means a molecule which is similar in at least one of structure or function relative to a starting molecule while a “heterologous molecule” is one that differs in at least one of structure or function relative to a starting molecule. Structural homologs are therefore molecules which are substantially structurally similar. They can be identical. Functional homologs are molecules which are substantially functionally similar. They can be identical.

Glycans, glycoproteins, antibodies and other products of the present invention may comprise conjugates. Such conjugates of the invention may include a naturally occurring substance or ligand, such as a protein (e.g., human serum albumin (HSA), low-density lipoprotein (LDL), high-density lipoprotein (HDL), or globulin); a carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Conjugates can also include targeting groups, e.g., a cell or tissue targeting agent or group, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine, N-acetyl-glucosamine multivalent mannose, multivalent fiucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Targeting groups may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, multivalent fucose, or aptamers.

The targeting group can be any ligand that is capable of targeting a specific receptor. Examples include, without limitation, folate, GalNAc, galactose, mannose, mannose-6P, aptamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein.

In still other embodiments, glycans, glycoproteins, antibodies and other products of the present invention are covalently conjugated to a cell penetrating polypeptide. The cell-penetrating peptide may also include a signal sequence. The conjugates of the invention can be designed to have increased stability; increased cell transfection; and/or altered biodistribution (e.g., targeted to specific tissues or cell types).

Conjugating moieties may be added to glycans, glycoproteins, antibodies and other products of the present invention such that they allow labeling or flagging targets for clearance. Such tagging/flagging molecules include, but are not limited to ubiquitin, fluorescent molecules, human influenza hemaglutinin (HA), c-myc (a 10 amino acid segment of the human protooncogene myc with sequence EQKLISEEDL), histidine (His), flag (a short peptide of sequence DYKDDDDK), glutathione S-transferase (GST), V5 (a paramyxovirus of simian virus 5 epitope), biotin, avidin, streptavidin, horse radish peroxidase (HRP) and digoxigenin.

In some embodiments, glycan-interacting antibodies may be combined with one another or other molecule in the treatment of a disease or condition.

### Nucleic Acids

The present invention embraces nucleic acid molecules. In some embodiments, nucleic acids encode antibodies of the invention (including, but not limited to antibodies, antibody fragments, intrabodies and chimeric receptor antigens). Such nucleic acid molecules include, without limitation, DNA molecules, RNA molecules, polynucleotides, oligonucleotides, mRNA molecules, vectors, plasmids and other constructs. As used herein, the term “construct” refers to any recombinant nucleic acid molecule including, but not limited to plasmids, cosmids, autonomously replicating polynucleotide molecules or linear or circular single-stranded or double-stranded DNA or RNA polynucleotide molecules. The present invention also embraces cells programmed or generated to express nucleic acid molecules encoding glycan-interacting antibodies. Such cells may be generated through the use of transfection, electroporation, viral delivery and the like. Viruses engineered with constructs of the invention may include, but are not limited to lentiviruses, adenoviruses, adeno-associated viruses and phages. In some cases, nucleic acids of the invention include codon-optimized nucleic acids. Methods of generating codon-optimized nucleic acids are known in the art and may include, but are not limited to those described in U.S. Pat. Nos. 5,786,464 and 6,114,148, the contents of each of which are herein incorporated by reference in their entirety.

### Epitope Characterization

Methods of the present invention may be used to characterize specific epitopes recognized by anti-glycan antibodies. Anti-glycan antibody epitopes may comprise a region on an antigen or between two or more antigens that is specifically recognized and bound by a corresponding antibody. Some epitopes may comprise one or more sugar residues. Such sugar residues may be part of one or more glycan. Such epitopes may comprise 1, 2, 3, 4, 5, 6, 7, 8, 9 or at least 10 sugar residues. In some cases, epitopes may comprise a partial sugar residue. Such epitopes may include one or more chemical groups of a sugar residue. In some cases, epitopes may comprise one or more regions of interaction between entities. In some embodiments, epitopes may comprise a junction between two sugar residues, between a branching chain and a parent chain or between a glycan and a protein. In some cases, epitopes may comprise chemical groups from two or more sugar residues. In some cases, epitopes may comprise a combination of chemical groups from one or more sugar residues and one or more non-sugar structure (e.g. amino acid, protein or post-translational protein modification). In some cases, epitopes may be formed by clustering of two or more glycans (e.g. the clustering of STn on a glycoprotein, glycoprotein complex or cell surface).

In some cases, epitope characterization may involve the use of one or more methods of analysis including, but not limited to binding assays, immunological assays, ELISAs, glycan arrays, Western Blots, surface plasmon resonance (SPR)-based assays, enzyme activity assays, mass spectrometry, X-ray crystallography, and genetic analysis.

In some embodiments, array glycans or glycans used in other epitope characterization assays may be modified to enhance the information gained with regard to one or more epitopes. In some cases, epitope modification may be useful when characterizing the epitope for one or more antibodies. In such cases, antibody binding may be assessed with or without epitope modification to provide information about the identity of an antibody's preferred epitope.

### Chemical Modification

In some cases, epitopes may be characterized using chemical modification. Antibody binding may be compared between modified and unmodified epitopes to determine the effect of such chemical modifications on antibody binding. In one example, periodate treatment may be used to chemically modify epitopes, including those comprising sialic acid. Periodate treatment may be used to destroy the C6 side chain of sialic acids. Assays may be conducted with and without periodate treatment to reveal whether or not anti-glycan antibody binding in untreated samples is sialic acid-specific.

The loss of O-acetylation on STn is relevant to cancer as cancer-associated expression correlates with increased STn recognition by antibodies (Ogata, S. et al., Tumor-associated sialylated antigens are constitutively expressed in normal human colonic mucosa. Cancer Res. 1995 May 1; 55(9):1869-74). In some cases, epitopes comprising 9-O-acetylated sialic acid may be chemically modified to destroy 9-O-acetyl groups. Such chemical modifications may be carried out through mild base treatment (e.g. with 0.1 M NaOH). Epitope characterization may be carried out with and without mild base treatment to reveal whether or not anti-glycan antibody binding in untreated samples depends on 9-O-acetylation of sialic acid.

In some cases, chemical modifications useful in epitope characterization may include oxidation reactions. Epitope oxidation may in some cases be carried out with one or more oxidizing agents. Oxidizing agents, may include, but are not limited to Tollen's reagent and Fehling's reagent. Epitope oxidation may alter binding of anti-glycan antibodies and provide insight into whether or not oxidized chemical groups are important for epitope interaction with such antibodies.

In some cases, chemical modifications useful in epitope characterization may include reduction reactions. Epitope reduction is typically carried out using one or more reducing agent. Reducing agents may include, but are not limited to one or more forms of borohydride. In some cases, reduction may comprise the conversion of CHO chemical groups to CH₂OH chemical groups. Epitope reduction may alter binding of anti-glycan antibodies and provide insight into whether or not reduced chemical groups are important for epitope interaction with such antibodies.

In some cases, chemical modifications useful in epitope characterization may include methylation. Epitope methylation may alter binding of anti-glycan antibodies and provide insight into whether or not methylated chemical groups are important for epitope interaction with such antibodies.

In some cases, chemical modifications useful in epitope characterization may include sulfation. Epitope sulfation may alter binding of anti-glycan antibodies and provide insight into whether or not the chemical groups being sulfated are important for epitope interaction with such antibodies.

In some cases, ether formation may be used as a chemical modification for epitope characterization. Ether formation may include the substitution of a hydrogen atom on one or more hydroxyl groups with a methyl group to form a methyl ether (—OH to —OCH₃). In one example, glycans are treated with an alkylating agent in the presence of a base to convert hydroxyl groups to methyl ether groups. In some cases, such alkylating agents may include, but are not limited to trimethylsulfoxonium iodide. Bases that may be used during such chemical modifications may include, but are not limited to sodium hydride (NaH). Epitope modification by ether formation may alter binding of anti-glycan antibodies and provide insight into whether or not the modified chemical groups are important for epitope interaction with such antibodies.

### Array Treatments

In some cases, glycan arrays of the invention may be subjected to chemical modification. Antibody binding may be compared between modified and unmodified glycans to determine the effect of such chemical modifications on antibody binding. In one example, periodate treatment may be used to chemically modify array glycans, including those comprising sialic acid. Periodate treatment may be used to destroy the C6 side chain of sialic acids. Assays may be conducted with and without periodate treatment to reveal whether or not anti-glycan antibody binding to array glycans is sialic acid-specific.

The loss of O-acetylation on STn is relevant to cancer as cancer-associated expression correlates with increased STn recognition by antibodies (Ogata, S. et al., Tumor-associated sialylated antigens are constitutively expressed in normal human colonic mucosa. Cancer Res. 1995 May 1; 55(9):1869-74). In some cases, array glycans comprising 9-O-acetylated sialic acid may be chemically modified to destroy 9-O-acetyl groups. Such chemical modifications may be carried out through mild base treatment (e.g. with 0.1 M NaOH). Antibody binding to array glycans may be carried out with and without mild base treatment to reveal whether or not anti-glycan antibody binding in untreated samples depends on 9-O-acetylation of sialic acid.

In some cases, chemical modifications useful in conjunction with glycan arrays may include oxidation reactions. Glycan oxidation may in some cases be carried out with one or more oxidizing agents. Oxidizing agents, may include, but are not limited to Tollen's reagent and Fehling's reagent. Array glycan oxidation may alter binding of anti-glycan antibodies and provide insight into whether or not oxidized chemical groups are important for array glycan interactions with such antibodies.

In some cases, chemical modifications useful glycan array analysis may include reduction reactions. Array glycan reduction is typically carried out using one or more reducing agents. Reducing agents may include, but are not limited to one or more forms of borohydride. In some cases, reduction may comprise the conversion of CHO chemical groups to CH₂OH chemical groups. Array glycan reduction may alter binding of anti-glycan antibodies and provide insight into whether or not reduced chemical groups are important for array glycan interaction with such antibodies.

In some cases, chemical modifications useful glycan array analysis may include methylation. Array glycan methylation may alter binding of anti-glycan antibodies and provide insight into whether or not methylated chemical groups are important for array glycan interaction with such antibodies.

In some cases, chemical modifications useful in glycan array analysis may include sulfation. Array glycan sulfation may alter binding of anti-glycan antibodies and provide insight into whether or not the chemical groups being sulfated are important for array glycan interaction with such antibodies.

In some cases, ether formation may be used as a chemical modification in glycan array analysis. Ether formation may include the substitution of a hydrogen atom on one or more hydroxyl groups with a methyl group to form a methyl ether (—OH to —OCH₃). In one example, glycans are treated with an alkylating agent in the presence of a base to convert hydroxyl groups to methyl ether groups. In some cases, such alkylating agents may include, but are not limited to trimethylsulfoxonium iodide. Bases that may be used during such chemical modifications may include, but are not limited to sodium hydride (NaH). Array glycan modification by ether formation may alter binding of anti-glycan antibodies and provide insight into whether or not the modified chemical groups are important for array glycan interaction with such antibodies.

### Three-Dimensional Epitope Assessment

In some cases, the three-dimensional structure of epitopes of the invention may be assessed. Antibodies sometimes recognize conformational, discontinuous epitopes. The three dimensional structure of epitopes may be determined by employing techniques such as X-ray crystallography, NMR spectroscopy, circular dichroism, vibrational spectroscopy and dual polarization interferometry.

In some cases, X-ray crystallography may be used to characterize epitopes at the atomic level. Such analysis may be used to determine the exact amino acid residues that interact between antibodies of the invention and their binding partners. Further variables determined by this analysis may include atomic distances. The results of X-ray crystallographic analysis may be used to further optimize antibodies of the invention by identifying potential regions for amino acid substitution.

Structural epitopes may be determined by nuclear magnetic resonance (NMR) spectroscopy techniques. NMR spectroscopy may be used to obtain information about the structure and dynamics of peptides, such as the quantum mechanical properties of the nucleus of the atom. These properties depend on the local molecular environment and their measurement provides information of the environment of atoms within the protein and such information in turn can be used to determine the overall three dimensional structure of epitopes.

Circular dichroism (CD) relies on the differential absorption of left and right circularly polarized radiation by chromophores. Proteins possess a number of chromophores which can give rise to CD signals, and which correspond to peptide bond absorption. CD spectrum obtained from an epitope (e.g. a peptide) can be analyzed for secondary structural features such as alpha-helix and beta-sheet and can provide information of the environments of the aromatic amino acid side chains for the tertiary structure of the epitope (see Kelly et al., Biochimia Biophysica Acta, 2005, 119-139, the contents of which are herein incorporated by reference in their entirety).

In further cases, vibrational spectroscopy and cryoelectron microscopy may also be used to determine the three dimensional structure of epitopes.

In other cases, conformational epitopes may be assessed based on computer based prediction using physicochemical features within the three dimensional structure of target proteins, such as the surface patch and consensus sequences (Liang et al., BMC Bioinformatics, 2009, 10, 302). Many machine learning methods have been developed to predict three dimensional structure of epitopes such as ElliPro (Ponomarenko et al., BMC Bioinformatics., 2008, 9, 514); SEPPA (Sun et al., Nucl. Acids Res., 2009, 37, 612-616); and Patchdock and SymmDock (Schneidman-Duhovny et al., Nucl. Acids Res 2005, 33, 363-367).

### Diagnostics

In some embodiments, the present invention provides methods of diagnosing one or more disease, disorder, and/or condition described herein. Such methods may include the use of an anti-glycan antibody profile obtained according to the methods described herein. In some cases, methods of diagnosing diseases, disorder, and/or conditions described herein may include the use of a glycan profile obtained according to the methods described herein. In some cases, these anti-glycan antibody profiles or glycan profiles may be obtained using a diagnostic kit of the invention.

Diseases, disorders, and/or conditions diagnosed according to methods of the invention may include, but are not limited to cancer or cancer-related indications; immune-related indications; viral indications; cardiovascular indications; and gastrointestinal indications. Cancer or cancer-related indications that may be diagnosed in a subject according to methods of the invention may include, but are not limited to cancers or cancer-related indications characterized by cells comprising one or more TACA in such subjects or characterized by the presence of one or more anti-TACA antibodies detected in such subjects.

In some embodiments, diagnostic arrays are prepared. As used herein, the term “diagnostic array” refers to an array used in the diagnosis of a disease, disorder, and/or condition. Diagnostic arrays of the invention may include, but are not limited to, glycan arrays and anti-glycan arrays. In some cases, diagnostic arrays of the invention may be used to diagnose cancer in a subject sample by detecting the presence of one or more anti-glycan antibodies. Such anti-glycan antibodies may include anti-STn antibodies.

Diagnostic arrays of the invention may be designed based on glycan array profiles obtained from a subject or tissue. In some cases, diagnostic arrays are prepared based on glycan array profiles obtained from a subject with cancer. In some cases, diagnostic arrays are prepared based on glycan array profiles obtained from a tumor or cancerous tissue. These glycan profiles may indicate the presence of one or more chemical groups associated with glycans associated with a tumor or cancerous tissue. Other glycan profiles may indicate the density of glycans associated with a tumor or cancerous tissue.

Diagnostic arrays may be optimized for detection of antibodies in a subject sample, wherein the antibodies are specific for glycans associated with a tumor or cancerous tissue that is characterized by the presence or absence of specific chemical groups. Such chemical groups may include, but are not limited to, 9-O acetyl chemical groups. In some embodiments, diagnostic arrays may be printed using a pH-optimized printing buffer. As used herein, a “pH-optimized printing buffer” refers to a printing buffer in which the pH has been adjusted to stabilize or destabilize at least one chemical group associated with glycans present in such pH-optimized printing buffer. In some cases, pH-optimized printing buffer may be prepared to have a pH of from about 4.0 to about 6.5, from about 5.0 to about 7.0, from about 6.0 to about 9.0, from about 6.5 to about 7.5, from about 7.4 to about 8.4, from about 8.0 to about 10.0, or from about 8.4 to about 12.0.

Methods of preparing diagnostic arrays may include the steps of: (1) obtaining a glycan profile of a tumor or cancerous tissue; (2) selecting at least one glycan according to the glycan profile; (3) preparing a pH-optimized printing buffer; and (4) preparing an array with selected glycans and the pH-optimized printing buffer.

In some embodiments, diagnostic arrays may be optimized for detection of antibodies in a subject sample, wherein the antibodies are specific for glycans associated with a tumor or cancerous tissue, wherein the tumor or cancerous tissue glycans have a density that is characteristic of that tumor or cancerous tissue. Tumor or cancerous tissue-specific glycans may have varying glycan densities that create distinct epitopes unique to glycans at those densities (e.g., interglycan epitopes or individual glycan epitopes wherein individual glycans adopt a specific conformation depending on the density of surrounding glycans). To detect antibodies specific for such density-dependent glycan epitopes, optimized diagnostic arrays may be printed using a glycan density-optimized printing buffer. As used herein, a “glycan density-optimized printing buffer” refers to a printing buffer in which the concentration of glycans present in the printing buffer has been adjusted to influence the density of glycans ultimately formed on arrays printed with such printing buffer. The concentration of glycans present in density-optimized printing buffer may be from about 1 μM to about 10 μM, from about 5 μM to about 25 μM, from about 20 μM to about 60 μM, from about 50 μM to about 100 μM, from about 75 μM to about 150 μM, from about 100 μM to about 300 μM, from about 200 μM to about 500 μM, or from about 250 μM to about 1 mM. In some cases, STn glycans may be used.

Methods of preparing a diagnostic array may include: (1) obtaining a glycan profile of a tumor or cancerous tissue, wherein the glycan density of the identified glycans is determined; (2) selecting at least one glycan identified by the glycan profile; (3) preparing a glycan density-optimized array, wherein the glycan density-optimized array is prepared by preparing a glycan density-optimized printing buffer prepared by adjusting the glycan concentration of the identified glycan(s) in the printing buffer; and (4) preparing a diagnostic array using the glycan density-optimized printing buffer.

### Pathogen Glycoprofiling

Many pathogenic bacteria produce glycan-binding proteins, including, but not limited to lectins, adhesins as well as some toxins. In some cases, such glycan-binding proteins may be capable of binding host glycans with a high degree of specificity (Topin, J. et al. 2013. PLoS One. 8(8): e71149). Further pathogen-associated glycans are described hereinabove.

In some embodiments, glycoprofiling methods of the present invention may be used to identify one or more pathogens that produce or present one or more glycans. In some cases, one or more binding assay of the present invention may be used. In some cases, arrays of the present invention may be used to identify one or more pathogens and/or identify one or more glycan-binding proteins produced by one or more pathogens. In some cases, pathogens and/or pathogen-derived glycan-binding proteins capable of binding one or more blood group antigen may be identified. Such antigens may include, but are not limited to human A, B and H antigens [corresponding to blood groups A, B or O, respectively (Topin, J. et al. 2013. PLoS One. 8(8): e71149).]

In some cases, glycoprofiling methods of the invention may be used to develop one or more antibodies directed to one or more glycans associated with one or more pathogens. In some cases, such methods may comprise the use of one or more binding assays. Such binding assays may include, but are not limited to glycan arrays, immunological assays, surface plasmon resonance and flow cytometry. In some cases, glycan arrays may be constructed to present a library of pathogen-associated glycans. Such glycan arrays may be contacted with antibody fragment display libraries and/or samples from immunized hosts or cell culture media containing one or more antibodies.

### Cancer Profiling

In some embodiments, glycoprofiling according to the present invention, may be used to identify an individual with cancer. This may involve the identification of one or more glycans in one or more samples obtained from such individuals. In some cases, glycoprofiling may be used to identify a particular type of cancer based on identification of cancer-specific glycans present on cancerous cells, in the area around a cancerous cell or in one or more fluid samples taken from a subject. Such methods may involve the use of one or more binding assays to identify one or more TACA. Such binding assays may include, but are not limited to glycan arrays, immunological assays, surface plasmon resonance and flow cytometry. In some cases, glycan arrays may be constructed to present a library of anti-TACA antibodies to bind one or more TACA present in a sample. In some cases, identifying an individual with cancer may comprise the use of one or more binding assays to identify one or more anti-TACA antibodies expressed by one or more subjects.

In some embodiments, glycoprofiling methods may be used to identify and/or develop one or more antibodies targeting one or more TACA. In some cases, such methods may comprise the use of one or more binding assays. Such binding assays may include, but are not limited to glycan arrays, immunological assays, surface plasmon resonance and flow cytometry. In some cases, glycan arrays may be constructed to present a library of glycans associated with one or more types of cancer or one or more tumor cells.

### Therapeutic Areas

### Cancer

Cancerous cells may present unique glycan epitopes on their cell surfaces (Varki, A. et al., 2009. Essentials of Glycobiology. 2^{nd} edition. Chapter 44). Such epitopes are excellent targets for cancer cell identification and targeting. Methods of the present invention may be used to diagnose, profile and/or treat subjects comprising such cancerous cells and/or circulating antibodies directed to glycan epitopes of cancerous cells. In some cases, such methods may include the use of one or more anti-glycan profiles, glycan profiles, kits and/or antibodies of the invention.

In some cases, cancer cells may present elevated levels of sialic acid in comparison to other and/or surrounding cells. In humans, as well as other species that are not capable of synthesizing Neu5Gc, dietary Neu5Gc may be incorporated at a higher levels and/or rate in cancerous cells. Such cancerous cells may thus present glycan epitopes comprising Neu5Gc on their surface that may be detected using products and/or methods of the present invention. Such glycan epitopes may include, but are not limited to any of those listed in Padler-Karvani et al., 2011. Cancer Res. 71:3352-63, the contents of which are herein incorporated by reference in their entirety. Additionally, such subjects may comprise circulating antibodies directed to glycan epitopes comprising Neu5Gc and/or elevated levels of such antibodies in relation to subjects without cancer.

In some embodiments, methods of the invention may be used to assess or target cancer-related antigens or epitopes. As used herein, the term “cancer-related” is used to describe entities that may be in some way associated with cancer, cancerous cells and/or cancerous tissues. Many cancer-related antigens or epitopes comprising glycans have been identified that are expressed in correlation with tumor cells (Heimburg-Molinaro, J. et al., Cancer vaccines and carbohydrate epitopes. Vaccine. 2011 Nov. 8; 29(48):8802-26). These are referred to herein as “tumor-associated carbohydrate antigens” or “TACAs.” TACAs include, but are not limited to mucin-related antigens [including, but not limited to Tn, Sialyl Tn (STn) and Thomsen-Friedenreich antigen], blood group Lewis related antigens [including, but not limited to Lewis^{Y} (Le^{Y}), Lewis^{X} (Le^{X}), Sialyl Lewis^{X} (SLe^{X}) and Sialyl Lewis^{A}(SLe^{A})], glycosphingolipid-related antigens [including, but not limited to Globo H, stage-specific embryonic antigen-3 (SSEA-3) and glycosphingolipids comprising sialic acid], ganglioside-related antigens [including, but not limited to gangliosides GD2, GD3, GM2, fucosyl GM1 and Neu5GcGM3] and polysialic acid-related antigens. Many of such antigens are described in International Patent Application No. PCT/US2011/021387, the contents of which are herein incorporated by reference in their entirety.

In some embodiments, TACA targets of the present invention include Lewis blood group antigens. Lewis blood group antigens comprise a fucose residue linked to GlcNAc by an al-3 linkage or an al-4 linkage. They may be found on both glycolipids and glycoproteins. Lewis blood group antigens may be found in the body fluid of individuals that are secretors of these antigens. Their appearance on red cells is due to absorption of Lewis antigens from the serum by the red cells.

In some embodiments, TACA targets of the present invention comprise Le^{Y}. Le^{Y} (also known as CD174) is made up of Galβ1,4GlcNAC comprising α1,2- as well as α1,3-linked fiucose residues yielding the Fucα(1,2)Galβ(1,4)Fucα(1,3)GlcNAc epitope. It is synthesized from the H antigen by α1,3 fucosyltransferases which attach the α1,3 fucose to the GlcNAc residue of the parent chain. Le^{Y} may be expressed in a variety of cancers including, but not limited to ovarian, breast, prostate, colon, lung and epithelial. Due to its low expression level in normal tissues and elevated expression level in many cancers, the Le^{Y} antigen is an attractive target for therapeutic antibodies.

In some embodiments, TACA targets of the present invention comprise Le^{X}. Le^{X} comprises the epitope Galβ1-4(Fucα1-3)GlcNAcβ-R. It is also known as CD15 and stage-specific embryonic antigen-1 (SSEA-1). This antigen was first recognized as being immunoreactive with sera taken from a mouse subjected to immunization with F9 teratocarcinoma cells. Le^{X} was also found to correlate with embryonic development at specific stages. It is also expressed in a variety of tissues both in the presence and absence of cancer, but can also be found in breast and ovarian cancers where it is only expressed by cancerous cells.

In some embodiments, TACA targets of the present invention comprise SLe^{A} and/or SLe^{X}. SLe^{A} and SLe^{X} comprise the structures [Neu5Acα2-3Galβ1-3(Fucα1-4)GlcNAcβ-R] and [Neu5Acα2-3Galβ1-4(Fucα1-3)GlcNAcβ-R] respectively. Their expression is upregulated in cancer cells. The presence of these antigens in serum correlates with malignancy and poor prognosis. SLe^{X} is mostly found as a mucin terminal epitope. It is expressed in a number of different cancers including breast, ovarian, melanoma, colon, liver, lung and prostate. In some embodiments of the present invention, SLe^{A} and SLe^{X} targets comprise Neu5Gc (referred to herein as GcSLe^{A} and GcSLe^{X}, respectively).

In some embodiments, TACA targets of the present invention comprise glycolipids and/or epitopes present on glycolipids, including, but not limited to glycosphingolipids. Glycosphingolipids comprise the lipid ceramide linked to a glycan by the ceramide hydroxyl group. On the cell membrane, glycosphingolipids form clusters referred to as “lipid rafts”.

In some embodiments, TACA targets of the present invention comprise Globo H. Globo H is a cancer-related glycosphingolipid first identified in breast cancer cells. The glycan portion of Globo H comprises Fucα(1-2)Galβ(1-3)GalNAcβ(1-3)Galα(1-4)Galβ(1-4)Glcβ(1). Although found in a number of normal epithelial tissues, Globo H has been identified in association with many tumor tissues including, but not limited to, small cell lung, breast, prostate, lung, pancreatic, gastric, ovarian and endometrial tumors.

In some embodiments, cancer-related glycosphingolipid targets of the present invention include gangliosides. Gangliosides are glycosphingolipids comprising sialic acid. According to ganglioside nomenclature, G is used as an abbreviation for ganglioside. This abbreviation is followed by the letters M, D or T referring to the number of sialic acid residues attached (1, 2 or 3 respectively). Finally the numbers 1, 2 or 3 are used to refer to the order of the distance each migrates when analyzed by thin layer chromatography (wherein 3 travels the greatest distance, followed by 2 and then 1). Gangliosides are known to be involved in cancer-related growth and metastasis and are expressed on the cell surface of tumor cells. Gangliosides expressed on tumor cells include, but are not limited to GD2, GD3, GM2 and fucosyl GM1 (also referred to herein as Fuc-GM1). In some embodiments of the present invention, glycan-interacting antibodies are directed toward GD3. GD3 is a regulator of cell growth. In some embodiments, GD3-directed antibodies are used to modulate cell growth and/or angiogenesis. In some embodiments, GD3-directed antibodies are used to modulate cell attachment. GD3 associated with some tumor cells may comprise 9-O-acetylated sialic acid residues (Mukherjee, K. et al., 2008. J Cell Biochem. 105: 724-34 and Mukherjee, K. et al., 2009. Biol Chem. 390: 325-35, the contents of each of which are herein incorporated by reference in their entirety). In some cases, antibodies of the invention are selective for 9-O-acetylated sialic acid residues. Some antibodies may be specific for 9-O-acetylated GD3s. Such antibodies may be used to target tumor cells expressing 9-O-acetylated GD3. In some embodiments of the present invention, glycan interacting antibodies are directed toward GM2. In some embodiments, GM2-directed antibodies are used to modulate cell to cell contact. In some embodiments, ganglioside targets of the present invention comprise Neu5Gc. In some embodiments, such targets may include a GM3 variant comprising Neu5Gc (referred to herein as GcGM3). The glycan component of GcGM3 is Neu5Gcα2-3Galβ1-4Glc. GcGM3 is a known component of tumor cells (Casadesus, A. V. et al., 2013. Glycoconj J. 30(7):687-99, the contents of which are herein incorporated by reference in their entirety).

In some embodiments, tumor-associated carbohydrate antigens of the present invention comprise Neu5Gc.

### STn in Cancer

The immune system has multiple mechanisms for promoting anti-tumor cell immune activity including both innate and adaptive immune activity. As used herein, the term “anti-tumor cell immune activity” refers to any activity of the immune system that kills or prevents growth and/or proliferation of tumor cells. In some cases, anti-tumor immune activity includes recognition and tumor cell killing by natural killer (NK) cells and phagocytosis by macrophages. Adaptive anti-tumor immune responses include tumor antigen uptake and presentation by antigen presenting cells (APCs,) such as dendritic cells (DCs,) leading to modulation of T cell anti-tumor activity and/or expansion of B cells with secretion of tumor-specific antibodies. The binding of tumor-specific antibodies to tumors can lead to antibody-dependent cellular cytotoxicity (ADCC) and complement-dependent cytotoxicity (CDC) mechanisms of tumor cell death.

As used herein, the term “immune-resistant tumor cell” refers to a tumor cell that reduces or evades anti-tumor cell immune activity. Some studies indicate that the expression of STn (a known TACA) on tumor cell surfaces or secreted into the tumor cell microenvironment can promote tumor cell evasion of anti-tumor immune activity. As used herein, the term “tumor cell microenvironment” refers to any area adjacent to or surrounding a tumor cell. Such areas include, but are not limited to areas between tumor cells, between tumor and non-tumor cells, surrounding fluids and surrounding components of the extracellular matrix.

Sialylated mucins comprising STn were demonstrated by Ogata et al to reduce NK cell targeting of tumor cells (Ogata, S. et al., 1992. Canc. Res. 52:4741-6, the contents of which are herein incorporated by reference in their entirety). This study found that the presence of ovine, bovine and porcine submaxillary mucin (OSM, BSM and PSM, respectively) led to nearly one hundred percent inhibition of cytotoxicity (see Table 2 therein). Further studies by Jandus et al, demonstrate that some tumor cells can evade NK destruction due to the expression of sialoglycan ligands that can interact with NK cell siglec receptors, leading to NK inhibition (Jandus, C. et al., 2014, JCI. pii: 65899, the contents of which are herein incorporated by reference in their entirety).

Studies by Toda et al., demonstrate that STn may bind CD22 receptors on B cells, leading to decreased signal transduction and reduced B cell activation (Toda, M. et al., 2008. Biochem Biophys Res Commun. 372(1):45-50, the contents of which are herein incorporated by reference in their entirety). Dendritic cells (DCs) can affect adaptive immune activity by modulating T cell activity. Studies by Carrascal et al found that STn expression by bladder cancer cells induced tolerance in DCs, reducing their ability to induce anti-tumor cell immune activity in T cells (Carrascal, M A et al., 2014. Mol Oncol. pii: S1574-7891(14)00047-7, the contents of which are herein incorporated by reference in their entirety). These studies revealed that DCs coming into contact with STn-positive bladder cancer cells displayed a tolorigenic expression profile with low expression of CD80, CD86, IL-12 and TNF-α. Further, DCs were found to modulate regulatory T cells such that the T cells had low expression of IFNγ and high expression of FoxP3. Other studies by van Vliet and others, indicate that DC surface expression of macrophage galactose-type lectin (MGL) can lead to targeting of those cells to tumor tissues (van Vliet, S J., 2007. Amsterdam: Vrije Universiteit. p 1-232 and van Vliet, S J. et al., 2008. J Immunol. 181(5):3148-55, Nollau, P. et al., 2013. J Histochem Cytochem. 61(3):199-205, the contents of each of which are herein incorporated by reference in their entirety). DCs arriving at tissues due to MGL interactions may influence T helper (Th) cells in one of three ways. DCs can induce T cell tolerance, T cell immune activity or downregulation of effector T cells. MGL has been shown to bind to both AcSTn and GcSTn and the affinity has been analyzed in depth (Mortezai, N. et al., 2013. Glycobiology. 23(7):844-52, the contents of which are herein incorporated by reference in their entirety). Interestingly, MUC1 expression on tumors has been shown to lead to T cell tolerance, protecting tumor cells from immune eradication.

In some embodiments, antibodies of the present invention (including, but not limited to anti-STn antibodies) of the present invention may be used to treat subjects comprising one or more tumor cells expressing one or more TACAs. In some cases, antibodies (including, but not limited to anti-STn antibodies) of the invention may be used to increase anti-tumor cell immune activity toward tumor cells expressing STn. Such antibodies may increase the adaptive immune response and/or the innate immune response toward immune-resistant tumor cells. Some antibodies may be used to increase NK anti-tumor cell activity. Such antibodies may, in some cases, block the interaction between glycan receptors expressed on NK cells and STn glycans on cancer cells or in surrounding tissues.

In some embodiments, antibodies (including, but not limited to anti-STn antibodies) of the invention may be used to increase B cell anti-tumor cell activity. Such antibodies may reduce the interaction between CD22 receptors on B cells and STn glycans on cancer cells or in surrounding tissues. A study by Sjoberg et al. demonstrates that 9-O-acetylation of α2,6-linked sialic acids on glycoproteins also reduced interaction between B cell CD22 receptors and such glycoproteins (Sjoberg, E. R. et al. 1994. JCB. 126(2): 549-562). Another study by Shi et al. reveals that higher levels of 9-O-acetylated sialic acid residues on murine erythroleukemia cells makes these cells more susceptible to complement-mediated lysis (Shi, W-X. et al., 1996. J of Biol Chem. 271(49): 31526-32, the contents of which are herein incorporated by reference in their entirety). In some embodiments, anti-STn antibodies of the invention are capable of selectively binding non-9-O-acetylated STn, reducing overall STn binding, but reducing tumor cell growth and/or proliferation. (e.g., through increased B cell anti-tumor activity and increased complement-mediated tumor cell destruction). In some embodiments, antibodies (including, but not limited to anti-STn antibodies) of the invention may be used to increase DC anti-tumor activity. Such antibodies may be used to reduce DC tolerance to tumor cells. Reduced DC tolerance may comprise increasing DC expression of CD80, CD86, IL-12 and/or TNF-α. In some cases, DC anti-tumor cell activity may comprise promotion of T cell anti-tumor cell activity. Such antibodies may prevent binding between DC MGL and glycans expressed on or around cancer cells.

A study by Ibrahim et al. suggests that high levels of anti-STn antibodies along with endocrine therapy may increase overall survival and time to progression (TTP) in women with metastatic breast cancer (Ibrahim, N. K. et al., 2013. 4(7): 577-584, the contents of which are herein incorporated by reference in their entirety). In this study, anti-STn antibody levels were elevated after vaccination with STn linked to keyhole-limpet Hemocyanin (KLH). In some embodiments, antibodies (including, but not limited to anti-STn antibodies) of the invention may be used in combination with endocrine therapy (e.g. tamoxifen and/or an aromatase inhibitor).

### Immune-Related Targets

In some embodiments, methods of the present invention may be used to diagnose, profile and/or treat one or more immune-related indications. In some cases, such methods may include the use of one or more anti-glycan profiles, glycan profiles, kits and/or antibodies of the invention. In some embodiments, antibodies of the invention may be immunomodulatory antibodies. As used herein, an immunomodulatory antibody is an antibody that enhances or suppresses one or more immune function or pathway.

Many bacterial glycans are known to comprise sialic acid. In some cases, such glycans allow bacteria to evade the innate immune system of hosts, including, but not limited to humans. In one example, bacterial glycans inhibit alternate complement pathway activation through factor H recognition. In another example, bacterial glycans mask underlying residues that may be antigenic. Some bacterial glycans participate in cell signaling events through activation of inhibitory sialic acid binding Ig-like lectins (Siglecs) that dampen the immune response to entities comprising certain sialylated moieties (Chen, X. et al., Advances in the biology and chemistry of sialic acids. ACS Chem Biol. 2010 Feb. 19; 5(2):163-76). In some embodiments, antibodies of the present invention may be used to treat immune complications related to bacterial glycans.

Due to the foreign nature of Neu5Gc as described herein, some Neu5Gc glycans are immunogenic resulting in immune related destruction of cells and other entities where these glycans may be expressed. Such autoimmune destruction may be pathogenic. In some embodiments, antibodies may be used to treat patients suffering from autoimmune disorders related to Neu5Gc glycans.

In some embodiments, immunomodulatory antibodies of the invention may be used to promote or suppress T cell-mediated immunity. Such antibodies may interact with one or more glycans present on T cells, T cell-related proteins and/or on one or more other cell types that interact with T cells. Immunomodulatory antibodies that enhance T cell mediated immunity may be used to stimulate T cell mediated targeting of cancer cells.

In some tumors, infiltration by tumor-associated macrophages (TAMs) may lead to immunosuppression promoting tumor cell viability and growth. This is thought to be due to immunosuppressive cell signaling that occurs through interactions between myeloid C-type lectin receptors (CLRs) present on TAMs and tumor-associated mucins (Allavena, P. et al., Clin Dev Immunol. 2010; 2010:547179). In some embodiments, binding of immunomodulatory antibodies of the invention to one or more tumor-associated mucin or TACA prevents immunosuppressive cell signaling in TAMs.

### Anti-Viral Applications

In some embodiments, methods of the invention may be used to diagnose and/or treat one or more viral infections. In some cases, such methods may include the use of one or more diagnostic kits, profiles and/or antibodies of the invention. In some cases, methods may include the use of antibodies that target one or more viruses. Viral coat proteins and viral envelopes often comprise glycans, referred to herein as viral surface glycans. Such glycans may be targets of antibodies of the present invention. In some embodiments, viral surface glycans comprise sialyl-STn. In a further embodiment, viral surface glycans comprise GcSTn. Viruses that may be targeted by antibodies of the invention include, but are not limited to HIV, influenza, rhinovirus, varicella-zoster, rotavirus, herpes (e.g. types 1 and 2), hepatitis (e.g. types A, B, C, D and E), yellow fever and human papillomavirus.

### Veterinary Applications

It is contemplated that methods, profiles and/or antibodies of the invention will find utility in the area of veterinary care including the care and treatment of non-human vertebrates. As described herein, the term “non-human vertebrate” includes all vertebrates with the exception of Homo sapiens, including wild and domesticated species such as companion animals and livestock. Non-human vertebrates include mammals, such as alpaca, banteng, bison, camel, cat, cattle, deer, dog, donkey, gayal, goat, guinea pig, horse, llama, mule, pig, rabbit, reindeer, sheep water buffalo, and yak. Livestock includes domesticated animals raised in an agricultural setting to produce materials such as food, labor, and derived products such as fiber and chemicals. Generally, livestock includes all mammals, avians and fish having potential agricultural significance. In particular, four-legged slaughter animals include steers, heifers, cows, calves, bulls, cattle, swine and sheep.

### Bioprocessing

In some embodiments, methods and/or antibodies of the invention may be used for producing biological products in host cells. Such methods typically include contacting cells with one or more agent capable of modulating gene expression, or altering levels and/or types of glycans produced wherein such modulation or alteration enhances production of biological products. According to the present invention, bioprocessing methods may be improved by using one or more of the methods and/or antibodies presented herein. Bioprocessing methods may also be improved by supplementing, replacing or adding one or more antibodies provided by the present invention.

### Pharmaceutical Compositions

Pharmaceutical compositions described herein can be characterized by one or more of bioavailability, therapeutic window and/or volume of distribution.

### Bioavailability

Antibodies, when formulated into a composition with a delivery/formulation agent or vehicle as described herein, can exhibit an increase in bioavailability as compared to a composition lacking a delivery agent as described herein. As used herein, the term “bioavailability” refers to the systemic availability of a given amount of antibodies administered to a mammal. Bioavailability can be assessed by measuring the area under the curve (AUC) or the maximum serum or plasma concentration (Cₘₐₓ) of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, herein incorporated by reference.

The Cₘₐₓ value is the maximum concentration of the compound achieved in the serum or plasma of a mammal following administration of the compound to the mammal. The Cₘₐₓ value of a particular compound can be measured using methods known to those of ordinary skill in the art. The phrases “increasing bioavailability” or “improving the pharmacokinetics,” as used herein mean that the systemic availability of an antibody, measured as AUC, Cₘₐₓ, or Cₘᵢₙ in a mammal is greater, when co-administered with a delivery agent as described herein, than when such co-administration does not take place. In some embodiments, the bioavailability of the antibody can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Therapeutic Window

Antibodies, when formulated into a composition with a delivery agent as described herein, can exhibit an increase in the therapeutic window of the administered antibody composition as compared to the therapeutic window of the administered antibody composition lacking a delivery agent as described herein. As used herein “therapeutic window” refers to the range of plasma concentrations, or the range of levels of therapeutically active substance at the site of action, with a high probability of eliciting a therapeutic effect. In some embodiments, the therapeutic window of the antibody when co-administered with a delivery agent as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

Antibodies, when formulated into a composition with a delivery agent as described herein, can exhibit an improved volume of distribution (V_{dist}), e.g., reduced or targeted, relative to a composition lacking a delivery agent as described herein. The volume of distribution (V_{dist}) relates the amount of the drug in the body to the concentration of the drug in the blood or plasma. As used herein, the term “volume of distribution” refers to the fluid volume that would be required to contain the total amount of the drug in the body at the same concentration as in the blood or plasma: V_{dist} equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, V_{dist} can be used to determine a loading dose to achieve a steady state concentration. In some embodiments, the volume of distribution of the antibody when co-administered with a delivery agent as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

In some embodiments, antibodies comprise compositions and/or complexes in combination with one or more pharmaceutically acceptable excipients. Pharmaceutical compositions may optionally comprise one or more additional active substances, e.g. therapeutically and/or prophylactically active substances. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

In some embodiments, compositions are administered to humans, human patients or subjects. For the purposes of the present disclosure, the phrase “active ingredient” generally refers to antibodies to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, or at least 80% (w/w) active ingredient. In one embodiment, active ingredients are antibodies directed toward glycans.

### Formulation

Antibodies of the invention can be formulated using one or more excipients to: (1) increase stability; (2) increase cell permeability; (3) permit the sustained or delayed release (e.g., from a formulation of the antibody); and/or (4) alter the biodistribution (e.g., target the antibody to specific tissues or cell types). In addition to traditional excipients such as any and all solvents, dispersion media, diluents, or other liquid vehicles, dispersion or suspension aids, surface active agents, isotonic agents, thickening or emulsifying agents, preservatives, formulations of the present invention can include, without limitation, liposomes, lipid nanoparticles, polymers, lipoplexes, core-shell nanoparticles, peptides, proteins, cells transfected with the antibodies (e.g., for transplantation into a subject) and combinations thereof.

### Excipients

As used herein, the term “excipient” refers to any substance combined with a compound and/or composition of the invention before use. In some embodiments, excipients are inactive and used primarily as a carrier, diluent or vehicle for a compound and/or composition of the present invention. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, M D, 2006; incorporated herein by reference).

The use of a conventional excipient medium is contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [Span®60], sorbitan tristearate [Span®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER*188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Vehicles

### Liposomes, Lipoplexes and Lipid Nanoparticles

Antibodies of the present invention may be formulated using one or more liposomes, lipoplexes, or lipid nanoparticles. In one embodiment, pharmaceutical compositions comprising antibodies further comprise liposomes. Liposomes are artificially-prepared vesicles which may primarily comprise one or more lipid bilayers and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment such formulations may also be constructed or compositions altered such that they passively or actively are directed to different cell types in vivo.

Formulations can also be selectively targeted through expression of different ligands on their surface as exemplified by, but not limited by, folate, transferrin, N-acetylgalactosamine (GalNAc), and antibody targeted approaches.

Liposomes, lipoplexes, or lipid nanoparticles may be used to improve the efficacy of antibody function as these formulations may be able to increase cell transfection with antibodies. The liposomes, lipoplexes, or lipid nanoparticles may also be used to increase the stability of antibodies.

Liposomes that are specifically formulated for antibody cargo are prepared according to techniques known in the art, such as described by Eppstein et al. (Eppstein, D. A. et al., Biological activity of liposome-encapsulated murine interferon gamma is mediated by a cell membrane receptor. Proc Natl Acad Sci USA. 1985 June; 82(11):3688-92); Hwang et al. (Hwang, K. J. et al., Hepatic uptake and degradation of unilamellar sphingomyelin/cholesterol liposomes: a kinetic study. Proc Natl Acad Sci USA. 1980 July; 77(7):4030-4); U.S. Pat. Nos. 4,485,045 and 4,544,545. Production of liposomes with sustained circulation time is also described in U.S. Pat. No. 5,013,556.

Liposomes comprising antibodies of the present invention may be generated using reverse phase evaporation utilizing lipids such as phosphatidylcholine, cholesterol as well as phosphatidylethanolamine that has been polyethylene glycol-derivatized. Filters with defined pore size are used to extrude liposomes of the desired diameter. In another embodiment, antibodies of the present invention can be conjugated to the external surface of liposomes by disulfide interchange reaction as is described by Martin et al. (Martin, F. J. et al., Irreversible coupling of immunoglobulin fragments to preformed vesicles. An improved method for liposome targeting. J Biol Chem. 1982 Jan. 10; 257(1):286-8).

### Polymers and Nanoparticles

Antibodies of the invention can be formulated using natural and/or synthetic polymers. Non-limiting examples of polymers which may be used for delivery include, but are not limited to DMRI/DOPE, poloxamer, chitosan, cyclodextrin, and poly(lactic-co-glycolic acid) (PLGA) polymers. These may be biodegradable.

The polymer formulation can permit the sustained or delayed release of antibodies (e.g., following intramuscular or subcutaneous injection). The altered release profile for antibodies can result in, for example, release of the antibodies over an extended period of time. The polymer formulation may also be used to increase the stability of antibodies.

Polymer formulations can also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009 6:659-668; Davis, Nature 2010 464:1067-1070; herein incorporated by reference in its entirety).

Antibodies of the invention can also be formulated as nanoparticles using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so delivery of antibodies may be enhanced. For antibodies, systems based on poly(2-(methacryloyloxy)ethyl phosphorylcholine)-block-(2-(diisopropylamino)ethyl methacrylate), (PMPC-PDPA), a pH sensitive diblock copolymer that self-assembles to form nanometer-sized vesicles, also known as polymersomes, at physiological pH may be used. These polymersomes have been shown to successfully deliver relatively high antibody payloads within live cells. (Massignani, et al, Cellular delivery of antibodies: effective targeted subcellular imaging and new therapeutic tool. Nature Proceedings, May, 2010).

In one embodiment, a PEG-charge-conversional polymer (Pitella et al., Biomaterials. 2011 32:3106-3114) may be used to form a nanoparticle to deliver antibodies of the present invention. The PEG-charge-conversional polymer may improve upon the PEG-polyanion block copolymers by being cleaved into a polycation at acidic pH, thus enhancing endosomal escape.

The use of core-shell nanoparticles has additionally focused on a high-throughput approach to synthesize cationic cross-linked nanogel cores and various shells (Siegwart et al., Proc Natl Acad Sci USA. 2011 108:12996-13001). The complexation, delivery, and internalization of the polymeric nanoparticles can be precisely controlled by altering the chemical composition in both the core and shell components of the nanoparticle.

In one embodiment, matrices of poly(ethylene-co-vinyl acetate), are used to deliver antibodies of the invention. Such matrices are described in Nature Biotechnology 10, 1446-1449 (1992).

### Antibody Formulations

Antibodies of the invention may be formulated for intravenous administration or extravascular administration (Daugherty, et al., Formulation and delivery issues for monoclonal antibody therapeutics. Adv Drug Deliv Rev. 2006 Aug. 7; 58(5-6):686-706, US patent publication number 2011/0135570, all of which are incorporated herein in their entirety). Extravascular administration routes may include, but are not limited to subcutaneous administration, intraperitoneal administration, intracerebral administration, intraocular administration, intralesional administration, topical administration and intramuscular administration.

Antibody structures may be modified to improve their effectiveness as therapeutics. Improvements may include, but are not limited to improved thermodynamic stability, reduced Fc receptor binding properties and improved folding efficiency. Modifications may include, but are not limited to amino acid substitutions, glycosylation, palmitoylation and protein conjugation.

Antibodies may be formulated with antioxidants to reduce antibody oxidation. Antibodies may also be formulated with additives to reduce protein aggregation. Such additives may include, but are not limited to albumin, amino acids, sugars, urea, guanidinium chloride, polyalchohols, polymers (such as polyethylene glycol and dextrans), surfactants (including, but not limited to polysorbate 20 and polysorbate 80) or even other antibodies.

Antibodies of the present invention may be formulated to reduce the impact of water on antibody structure and function. Antibody preparations in such formulations may be may be lyophilized. Formulations subject to lyophilization may include carbohydrates or polyol compounds to protect and stabilize antibody structure. Such compounds include, but are not limited to sucrose, trehalose and mannitol.

Antibodies of the present invention may be formulated with polymers. In one embodiment, polymer formulations may contain hydrophobic polymers. Such polymers may be microspheres formulated with polylactide-co-glycolide through a solid-in-oil-in-water encapsulation method. Microspheres comprising ethylene-vinyl acetate copolymer are also contemplated for antibody delivery and may be used to extend the time course of antibody release at the site of delivery. In another embodiment, polymers may be aqueous gels. Such gels may, for example, comprise carboxymethylcellulose. Aqueous gels may also comprise hyaluronic acid hydrogel. Antibodies may be covalently linked to such gels through a hydrazone linkage that allows for sustained delivery in tissues, including but not limited to the tissues of the central nervous system.

### Peptide and Protein Formulations

Antibodies of the invention may be formulated with peptides and/or proteins. In one embodiment, peptides such as, but not limited to, cell penetrating peptides and proteins and peptides that enable intracellular delivery may be used to deliver pharmaceutical formulations. A non-limiting example of a cell penetrating peptide which may be used with the pharmaceutical formulations of the present invention includes a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al., Mol. Ther. 3(3):310-8 (2001); Langel, Cell-Penetrating Peptides: Processes and Applications (CRC Press, Boca Raton FL, 2002); El-Andaloussi et al., Curr. Pharm. Des. 11(28):3597-611 (2003); and Deshayes et al., Cell. Mol. Life Sci. 62(16):1839-49 (2005), all of which are incorporated herein by reference). The compositions can also be formulated to include a cell penetrating agent, e.g., liposomes, which enhance delivery of the compositions to the intracellular space. Antibodies of the invention may be complexed to peptides and/or proteins such as, but not limited to, peptides and/or proteins from Aileron Therapeutics (Cambridge, MA) and Permeon Biologics (Cambridge, MA) in order to enable intracellular delivery (Cronican et al., ACS Chem. Biol. 2010 5:747-752; McNaughton et al., Proc. Natl. Acad. Sci. USA 2009 106:6111-6116; Sawyer, Chem Biol Drug Des. 2009 73:3-6; Verdine and Hilinski, Methods Enzymol. 2012; 503:3-33; all of which are herein incorporated by reference in their entirety).

In one embodiment, the cell-penetrating polypeptide may comprise a first domain and a second domain. The first domain may comprise a supercharged polypeptide. The second domain may comprise a protein-binding partner. As used herein, “protein-binding partner” includes, but are not limited to, antibodies and functional fragments thereof, scaffold proteins, or peptides. The cell-penetrating polypeptide may further comprise an intracellular binding partner for the protein-binding partner. The cell-penetrating polypeptide may be capable of being secreted from a cell where antibodies may be introduced.

In formulations of the present invention, peptides or proteins may be incorporated to increase cell transfection by antibodies or alter the biodistribution of antibodies (e.g., by targeting specific tissues or cell types).

### Cell Formulations

Cell-based formulations of antibody compositions of the invention may be used to ensure cell transfection (e.g., in the cellular carrier) or alter the biodistribution of the compositions (e.g., by targeting the cell carrier to specific tissues or cell types).

### Cell Transfer Methods

A variety of methods are known in the art and are suitable for introduction of nucleic acids or proteins, such as antibodies, into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

The technique of sonoporation, or cellular sonication, is the use of sound (e.g., ultrasonic frequencies) for modifying the permeability of the cell plasma membrane. Sonoporation methods are known to those in the art and are used to deliver nucleic acids in vivo (Yoon and Park, Expert Opin Drug Deliv. 2010 7:321-330; Postema and Gilja, Curr Pharm Biotechnol. 2007 8:355-361; Newman and Bettinger, Gene Ther. 2007 14:465-475; all herein incorporated by reference in their entirety). Sonoporation methods are known in the art and are also taught for example as it relates to bacteria in US Patent Publication 20100196983 and as it relates to other cell types in, for example, US Patent Publication 20100009424, each of which are incorporated herein by reference in their entirety.

Electroporation techniques are also well known in the art and are used to deliver nucleic acids in vivo and clinically (Andre et al., Curr Gene Ther. 2010 10:267-280; Chiarella et al., Curr Gene Ther. 2010 10:281-286; Hojman, Curr Gene Ther. 2010 10:128-138; all herein incorporated by reference in their entirety). In one embodiment, antibodies may be delivered by electroporation.

### Administration and Delivery

The compositions of the present invention may be administered by any of the standard methods or routes known in the art.

Antibodies of the present invention may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier. Non-limiting routes of administration for antibodies of the present invention are described below.

### Parenteral and Injectable Administration

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR©, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Administration

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, compositions containing antibodies of the invention may be formulated for administration topically. The skin may be an ideal target site for delivery as it is readily accessible. Gene expression may be restricted not only to the skin, potentially avoiding nonspecific toxicity, but also to specific layers and cell types within the skin.

The site of cutaneous expression of the delivered compositions will depend on the route of nucleic acid delivery. Three routes are commonly considered to deliver antibodies to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions). Antibodies can be delivered to the skin by several different approaches known in the art.

In one embodiment, the invention provides for a variety of dressings (e.g., wound dressings) or bandages (e.g., adhesive bandages) for conveniently and/or effectively carrying out methods of the present invention. Typically dressing or bandages may comprise sufficient amounts of pharmaceutical compositions and/or antibodies described herein to allow a user to perform multiple treatments of a subject(s).

In one embodiment, the invention provides for compositions comprising antibodies to be delivered in more than one injection.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required.

Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, compositions of the present invention are formulated in depots for extended release. Generally, a specific organ or tissue (a “target tissue”) is targeted for administration.

In some aspects of the invention, antibodies are spatially retained within or proximal to a target tissue. Provided are methods of providing compositions to one or more target tissue of a mammalian subject by contacting the one or more target tissue (comprising one or more target cells) with compositions under conditions such that the compositions, in particular antibody component(s) of the compositions, are substantially retained in the target tissue, meaning that at least 10, 20, 30, 40, 50, 60, 70, 80, 85, 90, 95, 96, 97, 98, 99, 99.9, 99.99 or greater than 99.99% of the composition is retained in the target tissue. Advantageously, retention is determined by measuring the level of antibodies present in the compositions entering the target tissues and/or cells. For example, at least 1, 5, 10, 20, 30, 40, 50, 60, 70, 80, 85, 90, 95, 96, 97, 98, 99, 99.9, 99.99 or greater than 99.99% of antibodies administered to the subject are present intracellularly at a period of time following administration. For example, intramuscular injection to a mammalian subject is performed using an aqueous composition comprising one or more antibody and a transfection reagent, and retention of the composition is determined by measuring the level of antibodies present in the muscle cells.

Certain aspects of the invention are directed to methods of providing compositions to target tissues of mammalian subjects, by contacting the target tissues (containing one or more target cells) with compositions under conditions such that the compositions are substantially retained in the target tissue. Compositions contain an effective amount of antibodies such that the effect of interest is produced in at least one target cell. Compositions generally contain cell penetration agents and a pharmaceutically acceptable carrier, although “naked” antibodies (such as antibodies without cell penetration agents or other agents) are also contemplated.

In some embodiments, compositions include a plurality of different antibodies, where one or more than one of the antibodies targets a glycan of interest. Optionally, compositions also contain cell penetration agents to assist in the intracellular delivery of compositions. A determination is made of the composition dose required to target glycans of interest in a substantial percentage of cells contained within a predetermined volume of the target tissue (generally, without targeting glycans in tissue adjacent to the predetermined volume, or distally to target tissues). Subsequent to this determination, the determined dose may be introduced directly into the tissue of the mammalian subject.

In one embodiment, the invention provides for antibodies to be delivered in more than one injection or by split dose injections.

### Pulmonary Administration

Pharmaceutical compositions may be prepared, packaged, and/or sold in formulations suitable for pulmonary administration via the buccal cavity. Such formulations may comprise dry particles further comprising active ingredients and having a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal and Buccal Administration

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

### Ophthalmic or Otic Administration

A pharmaceutical composition may be prepared, packaged, and/or sold in a formulation suitable for ophthalmic or otic administration. Such formulations may, for example, be in the form of eye or ear drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Subretinal inserts may also be used as a form of administration.

### Payload Administration

Antibodies described herein may be used in a number of different scenarios in which delivery of a substance (the “payload”) to a biological target is desired, for example delivery of detectable substances for detection of the target, or delivery of a therapeutic or diagnostic agent. Detection methods can include, but are not limited to, both imaging in vitro and in vivo imaging methods, e.g., immunohistochemistry, bioluminescence imaging (BLI), Magnetic Resonance Imaging (MRI), positron emission tomography (PET), electron microscopy, X-ray computed tomography, Raman imaging, optical coherence tomography, absorption imaging, thermal imaging, fluorescence reflectance imaging, fluorescence microscopy, fluorescence molecular tomographic imaging, nuclear magnetic resonance imaging, X-ray imaging, ultrasound imaging, photoacoustic imaging, lab assays, or in any situation where tagging/staining/imaging is required.

Antibodies can be designed to include both a linker and a payload in any useful orientation. For example, a linker having two ends is used to attach one end to the payload and the other end to the antibody. The antibodies of the invention can include more than one payload as well as a cleavable linker. In another example, a drug that may be attached to antibodies via a linker and may be fluorescently labeled can be used to track the drug in vivo, e.g. intracellularly.

Other examples include, but are not limited to, the use of antibodies in reversible drug delivery into cells.

Antibodies described herein can be used in intracellular targeting of a payload, e.g., detectable or therapeutic agents, to specific organelles. In addition, antibodies described herein may be used to deliver therapeutic agents to cells or tissues, e.g., in living animals. For example, antibodies described herein may be used to deliver chemotherapeutic agents to kill cancer cells. Antibodies attached to therapeutic agents through linkers can facilitate member permeation allowing the therapeutic agent to travel into a cell to reach an intracellular target.

In some embodiments, the payload may be a therapeutic agent such as a cytotoxin, radioactive ion, chemotherapeutic, or other therapeutic agent. A cytotoxin or cytotoxic agent includes any agent that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020 incorporated herein in its entirety), rachelmycin (CC-1065, see U.S. Pat. Nos. 5,475,092, 5,585,499, and 5,846,545, all of which are incorporated herein by reference), and analogs or homologs thereof. Radioactive ions include, but are not limited to iodine (e.g., iodine 125 or iodine 131), strontium 89, phosphorous, palladium, cesium, iridium, phosphate, cobalt, yttrium 90, samarium 153, and praseodymium. Other therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, rachelmycin (CC-1065), melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). In the case of anti-STn antibodies of the present invention, tumor killing may be boosted by the conjugation of a toxin to such anti-STn antibodies.

In some embodiments, the payload may be a detectable agent, such as various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., ¹⁸F, ⁶⁷Ga, ^{81m}Kr, ⁸²Rb, ¹¹¹In, ¹²³I, ¹³³Xe, ²⁰¹Tl, ¹²⁵I, ³⁵S, ¹⁴C, ³H, or ^{99m}Tc (e.g., as pertechnetate (technetate(VII), TcO₄⁻)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons). Such optically-detectable labels include for example, without limitation, 4-acetamido-4′-isothiocyanatostilbene-2,2′disulfonic acid; acridine and derivatives (e.g., acridine and acridine isothiocyanate); 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-l-naphthyl)maleimide; anthranilamide; BODIPY; Brilliant Yellow; coumarin and derivatives (e.g., coumarin, 7-amino-4-methylcoumarin (AMC, Coumarin 120), and 7-amino-4-trifluoromethylcoumarin (Coumarin 151)); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]-naphthalene-1-sulfonyl chloride (DNS, dansylchloride); 4-dimethylaminophenylazophenyl-4′-isothiocyanate (DABITC); eosin and derivatives (e.g., eosin and eosin isothiocyanate); erythrosin and derivatives (e.g., erythrosin B and erythrosin isothiocyanate); ethidium; fluorescein and derivatives (e.g., 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, X-rhodamine-5-(and -6)-isothiocyanate (QFITC or XRITC), and fluorescamine); 2-[2-[3-[[1,3-dihydro-1,1-dimethyl-3-(3-sulfopropyl)-2H-benz[e]indol-2-ylidene]ethylidene]-2-[4-(ethoxycarbonyl)-1-piperazinyl]-1-cyclopenten-1-yl]ethenyl]-1,1-dimethyl-3-(3-sulforpropyl)-1H-benz[e]indolium hydroxide, inner salt, compound with n,n-diethylethanamine (1:1) (IR144); 5-chloro-2-[2-[3-[(5-chloro-3-ethyl-2(3H)-benzothiazol-ylidene)ethylidene]-2-(diphenylamino)-1-cyclopenten-1-yl]ethenyl]-3-ethyl benzothiazolium perchlorate (IR140); Malachite Green isothiocyanate; 4-methylumbelliferone orthocresolphthalein; nitrotyrosine; pararosaniline; Phenol Red; B-phycoerythrin; o-phthaldialdehyde; pyrene and derivatives (e.g., pyrene, pyrene butyrate, and succinimidyl 1-pyrene); butyrate quantum dots; Reactive Red 4 (CIBACRON™ Brilliant Red 3B-A); rhodamine and derivatives (e.g., 6-carboxy-X-rhodamine (ROX), 6-carboxyrhodamine (R6G), lissamine rhodamine B sulfonyl chloride rhodarnine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′tetramethyl-6-carboxyrhodamine (TAMRA) tetramethyl rhodamine, and tetramethyl rhodamine isothiocyanate (TRITC)); riboflavin; rosolic acid; terbium chelate derivatives; Cyanine-3 (Cy3); Cyanine-5 (Cy5); cyanine-5.5 (Cy5.5), Cyanine-7 (Cy7); IRD 700; IRD 800; Alexa 647; La Jolta Blue; phthalo cyanine; and naphthalo cyanine.

In some embodiments, the detectable agent may be a non-detectable precursor that becomes detectable upon activation (e.g., fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE® (VisEn Medical))). In vitro assays in which the enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis.

### Combinations

Antibodies may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, and/or imaging compositions in combination with agents that may improve their bioavailability, reduce and/or modify their metabolism, inhibit their excretion, and/or modify their distribution within the body.

### Dosage

The present disclosure encompasses delivery of antibodies for any of therapeutic, pharmaceutical, diagnostic or imaging by any appropriate route taking into consideration likely advances in the sciences of drug delivery. Delivery may be naked or formulated.

### Naked Delivery

Antibodies of the present invention may be delivered to cells, tissues, organs or organisms in naked form. As used herein in, the term “naked” refers to antibodies delivered free from agents or modifications which promote transfection or permeability. Naked antibodies may be delivered to cells, tissues, organs and/or organisms using routes of administration known in the art and described herein. Naked delivery may include formulation in a simple buffer such as saline or PBS.

### Formulated Delivery

Antibodies of the present invention may be formulated, using methods described herein. Formulations may comprise antibodies which may be modified and/or unmodified. Formulations may further include, but are not limited to, cell penetration agents, pharmaceutically acceptable carriers, delivery agents, bioerodible or biocompatible polymers, solvents, and sustained-release delivery depots. Formulated antibodies may be delivered to cells using routes of administration known in the art and described herein.

Compositions may also be formulated for direct delivery to organs or tissues in any of several ways in the art including, but not limited to, direct soaking or bathing, via a catheter, by gels, powder, ointments, creams, gels, lotions, and/or drops, by using substrates such as fabric or biodegradable materials coated or impregnated with compositions, and the like.

### Dosing

The present invention provides methods comprising administering one or more antibodies in accordance with the invention to a subject in need thereof. Nucleic acids encoding antibodies, proteins or complexes comprising antibodies, or pharmaceutical, imaging, diagnostic, or prophylactic compositions thereof, may be administered to a subject using any amount and any route of administration effective for preventing, treating, diagnosing, or imaging a disease, disorder, and/or condition. The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 0.0001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

According to the present invention, antibodies may be administered in split-dose regimens. As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in a 24 hr period. It may be administered as a single unit dose. In one embodiment, antibodies of the present invention are administered to a subject in split doses. Antibodies may be formulated in buffer only or in a formulation described herein. Pharmaceutical compositions comprising antibodies as described herein may be formulated into a dosage form described herein, such as a topical, intranasal, intratracheal, or injectable (e.g., intravenous, intraocular, intravitreal, intramuscular, intracardiac, intraperitoneal or subcutaneous). General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Kits

Any of the antibodies, glycans, arrays, assays or other compounds or components described herein may be comprised in a kit. In a non-limiting example, reagents for generating antibodies, including antigen molecules are included in a kit. The kit may further include reagents or instructions for creating or synthesizing antibodies. It may also include one or more buffers. Other kits of the invention may include components for making antibody protein or nucleic acid arrays or libraries and thus, may include, for example, a solid support.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. The kits may also comprise a second container means for containing a sterile, pharmaceutically acceptable buffer and/or other diluent. However, various combinations of components may be comprised in a vial. The kits of the present invention also will typically include a means for containing the antibodies, e.g., proteins, nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried powder. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least 1000 micrograms or at most 10 g of dried dye are provided in kits of the invention. The dye may then be resuspended in any suitable solvent, such as DMSO.

Some kits of the invention include diagnostic kits. Such kits may be designed for the diagnosis of one or more indication described herein. In some cases, diagnostic kits of the invention may be used for research or development purposes (e.g. in the development of antibodies).

A kit may include instructions for employing the kit components as well the use of any other reagent not included in the kit. Instructions may include variations that can be implemented.

### Definitions

Detectable label: As used herein, “detectable label” refers to one or more markers, signals, or moieties which are attached, incorporated or associated with another entity, which markers, signals or moieties are readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the entity with which they are attached, incorporated or associated. For example, when attached, incorporated in or associated with a peptide or protein, they may be within the amino acids, the peptides, or proteins, or located at the N- or C-termini.

Epitope: As used herein, an “epitope” refers to a surface or region on a molecule that interacts with components of the immune system, including, but not limited to antibodies.

Linker: As used herein, a “linker” refers to a moiety that connects two or more domains, moieties or entities or a moiety that links one or more domains, moieties or entities to a surface or substrate.

Pathogen: As used herein, a “pathogen” refers to any entity causing or contributing to one or more diseases, disorders and/or conditions. Exemplary pathogens may include, but are not limited to microorganisms, parasites, bacteria, viruses, fungi, protozoa and prions.

Sample: As used herein, the term “sample” refers to an aliquot or portion taken from a source and/or provided for analysis or processing. Sources may include in vitro sources and in vivo sources. In vitro sources may include, but are not limited to cultured cells, cell culture lysates and cell culture media. In vivo sources may include, but are not limited to human subjects and non-human animal subjects. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is or comprises a medium, such as a nutrient broth or gel, which may contain cellular components, such as proteins or nucleic acid molecule. Samples may comprise one or more proteins, in some cases, isolated from an extract, lysate or other preparation. Protein samples may be homogenous or heterogeneous with regard to protein and/or glycan composition.

Subject: As used herein, the term “subject” refers to any organism to which a composition in accordance with the invention may be administered, e.g., for experimental, diagnostic, prophylactic, and/or therapeutic purposes. Typical subjects include human subjects as well as non-human animal subjects (e.g., mice, rats, rabbits, cats, dogs, pigs, cows, sheep, chicken and monkeys) and/or plants.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1. Immunization Using Alternative Adjuvants, Antigens and Mouse Strains

An immunization study was carried out to develop mice with immune responses to sialylated antigens using enhanced adjuvants. 40 each of Cmah−/− (male and female, ˜6-8 weeks old) and C57BL/6 mice (females, 6-8 weeks old) were acclimated for at least 3 days and given access to standard diet (2920X.10, Global 18% Protein Rodent Diet, Harlan, San Diego, CA) and acidified water (pH 2.7-3.0) ad libitum throughout the study period. Mice from each strain (Cmah−/− and C57BL/6) were divided into 4 groups of 10 mice each (a total of 8 groups).

Mice were immunized according to the study design shown in the Table below using either PSM or OSM at doses of either 10 μg or 100 μg (from 1 mg/ml stock solution) depending on the adjuvant used. Adjuvants included either Freund's adjuvant (complete or incomplete) or enhanced adjuvants comprising AbiSCO-100 (12 μg) and ODN-2395 (100 μg). Mice were vaccinated on days 0, 14, 28, 42 and 56 of the study and blood was collected for antibody analysis prior to each vaccination. Mice receiving vaccinations with Freund's adjuvant received complete Freund's adjuvant (CFA) with their first vaccination and incomplete Freund's adjuvant (IFA) during subsequent vaccinations.

TABLE 2
Study Design

| Group | Strain | Immunogen and Adjuvant |
|---|---|---|
| 1 | Cmah-/- | PSM (100 μg) + CFA or IFA (100 μl) |
| 2 | Cmah-/- | PSM (10 μg) + AbiSCO-100 (12 μg) + ODN-2395 (100 μg) |
| 3 | Cmah-/- | OSM (100 μg) + CFA or IFA (100 μl) |
| 4 | Cmah-/- | OSM (10 μg) + AbiSCO-100 (12 μg) + ODN-2395 (100 μg) |
| 5 | C57BL/6 | PSM (100 μg) + CFA or IFA (100 μl) |
| 6 | C57BL/6 | PSM (10 μg) + AbiSCO-100 (12 μg) + ODN-2395 (100 μg) |
| 7 | C57BL/6 | OSM (100 μg) + CFA or IFA (100 μl) |
| 8 | C57BL/6 | OSM (10 μg) + AbiSCO-100 (12 μg) + ODN-2395 (100 μg) |

Mice were randomized for placement into individual treatment groups based on body weight and sex. Vaccinations were given by subcutaneous injections around armpits and inguinal regions (50 μl per site, 4 sites for a total of 200 μl per mouse). Additionally, body weight and health observations for each mouse were determined twice per week.

During each blood collection, approximately 0.2 ml of whole blood was collected by facial vein bleed and placed into serum separator tubes. Tubes were then kept at room temperature for at least 30 minutes to allow clotting. Serum was then divided into aliquots and stored at −80° C. until analysis. An additional blood collection was also carried out on day 66 of the study. Blood samples were processed to serum and kept on ice for analysis on the same day.

To determine the titer of anti-STn antibodies, mouse sera collected at day 42 was analyzed by EIA. Plates were coated with coating buffer (50 mM Na carbonate/bicarbonate, pH 9.5, Sigma-Aldrich, St. Louis, MO) containing 1 μg BSM/100 μl overnight at 4° C. The next day, plates were incubated with 0.1 M NaOH for 30 min at 37° C. before being washed with phosphate buffered saline (PBS, pH 7.3, Sigma-Aldrich, St. Louis, MO). Half of the wells in each plate were next treated with either PBS (pH 6.5) or periodate solution [2 mM NaIO₄ (MW=213.98 g/mol) in PBS, pH6.5; Sigma-Aldrich, St. Louis, MO] for 20 min in the dark with gentle shaking. Solutions were removed by washing with PBS (pH 7.4) and then incubated overnight at 4° C. in blocking solution (PBS with 0.1% powdered egg white).

Test samples as well as positive [comprising anti-STn antibody (from mouse hybridoma clone 3F1) from SBH Biosciences, Natick, MA] and negative control samples were prepared by generating serial dilutions in blocking buffer. Blocking solution was removed from blocked plates and sample dilutions were added to wells at a volume of 100 μl/well. Plates were then incubated for 2 hours at room temperature. After washing with PBS with 0.05% Tween-20, wells were treated with goat anti-mouse IgG-HRP (Jackson Immunoresearch Laboratories, Inc., West Grove, PA; 100 μl/well at a dilution of 1:5,000 in PBS). After a one hour incubation at room temperature, wells were washed with PBS with 0.05% Tween-20. To visualize bound secondary antibodies, wells were finally treated with 100 μl/well of HRP substrate. Reactions were stopped with 100 μl/well of 1.6 M sulfuric acid and optical density (OD) values for each well were obtained spectrophotometrically at 490 nm. The highest dilution of each sample tested to result in detectable levels of reaction product (adjusted mean optical density of 0.050 or greater) are listed in the Table below.

TABLE 3
Highest sample dilutions with detectable antibody

| | | Highest sample dilution | |
|---|---|---|---|
| | | with detectable antibody | |
| Group | Animal ID | Day0 | Day42 |
| 1 | #3094 | <1:100 | 1:2500 |
| 1 | #3095 | <1:100 | <1:100 |
| 1 | #3071 | <1:100 | 1:12500 |
| 1 | #3081 | <1:100 | 1:12500 |
| 1 | #3295 | <1:100 | 1:500 |
| 1 | #3099 | <1:100 | 1:2500 |
| 1 | #3083 | <1:100 | 1:12500 |
| 1 | #2793 | <1:100 | 1:100 |
| 1 | #2795 | <1:100 | 1:500 |
| 1 | #3087 | <1:100 | 1:12500 |
| 2 | #3091 | <1:100 | 1:12500 |
| 2 | #3092 | <1:100 | <1:100 |
| 2 | #3074 | <1:100 | 1:12500 |
| 2 | #3096 | <1:100 | 1:12500 |
| 2 | #2791 | <1:100 | 1:2500 |
| 2 | #2792 | <1:100 | 1:12500 |
| 2 | #3097 | <1:100 | <1:100 |
| 2 | #3088 | <1:100 | 1:62500 |
| 2 | #3298 | <1:100 | 1:500 |
| 2 | #2798 | <1:100 | 1:2500 |
| 3 | #3790 | <1:100 | 1:500 |
| 3 | #3090 | <1:100 | 1:12500 |
| 3 | #3084 | <1:100 | 1:2500 |
| 3 | #3082 | <1:100 | 1:500 |
| 3 | #3075 | <1:100 | 1:100 |
| 3 | #3297 | <1:100 | 1:500 |
| 3 | #3793 | <1:100 | 1:2500 |
| 3 | #3085 | <1:100 | 1:2500 |
| 3 | #3098 | <1:100 | 1:500 |
| 3 | #3089 | <1:100 | 1:500 |
| 4 | #3093 | <1:100 | 1:12500 |
| 4 | #3076 | <1:100 | 1:12500 |
| 4 | #3072 | <1:100 | 1:2500 |
| 4 | #3073 | <1:100 | 1:2500 |
| 4 | #3299 | <1:100 | 1:12500 |
| 4 | #3296 | <1:100 | 1:12500 |
| 4 | #3791 | <1:100 | 1:2500 |
| 4 | #2794 | <1:100 | 1:12500 |
| 4 | #3792 | <1:100 | 1:2500 |
| 4 | #2796 | <1:100 | 1:2500 |
| 5 | #4416 | <1:100 | 1:2500 |
| 5 | #4435 | <1:100 | 1:62500 |
| 5 | #4420 | <1:100 | 1:2500 |
| 5 | #4402 | <1:100 | 1:2500 |
| 5 | #4415 | <1:100 | <1:100 |
| 5 | #4439 | <1:100 | 1:100 |
| 5 | #4405 | <1:100 | <1:100 |
| 5 | #4433 | <1:100 | 1:100 |
| 5 | #4412 | <1:100 | 1:500 |
| 5 | #4426 | <1:100 | <1:100 |
| 6 | #4427 | <1:100 | 1:62500 |
| 6 | #4434 | <1:100 | 1:2500 |
| 6 | #4423 | <1:100 | 1:12500 |
| 6 | #4418 | <1:100 | 1:12500 |
| 6 | #4436 | <1:100 | 1:62500 |
| 6 | #4438 | <1:100 | 1:12500 |
| 6 | #4432 | <1:100 | <1:100 |
| 6 | #4421 | <1:100 | 1:2500 |
| 6 | #4428 | <1:100 | 1:2500 |
| 6 | #4401 | <1:100 | 1:12500 |
| 7 | #4419 | <1:100 | 1:2500 |
| 7 | #4413 | <1:100 | 1:100 |
| 7 | #4424 | <1:100 | 1:12500 |
| 7 | #4408 | <1:100 | 1:2500 |
| 7 | #4409 | <1:100 | 1:500 |
| 7 | #4417 | <1:100 | 1:2500 |
| 7 | #4437 | <1:100 | 1:100 |
| 7 | #4430 | <1:100 | 1:100 |
| 7 | #4425 | <1:100 | <1:100 |
| 7 | #4429 | <1:100 | <1:100 |
| 8 | #4407 | <1:100 | 1:62500 |
| 8 | #4406 | <1:100 | 1:12500 |
| 8 | #4440 | <1:100 | 1:12500 |
| 8 | #4403 | <1:100 | 1:12500 |
| 8 | #4411 | <1:100 | 1:62500 |
| 8 | #4414 | <1:100 | 1:62500 |
| 8 | #4431 | <1:100 | 1:62500 |
| 8 | #4422 | <1:100 | 1:12500 |
| 8 | #4404 | <1:100 | 1:62500 |
| 8 | #4410 | <1:100 | 1:62500 |

At day 42, the results indicated that group 8 mice, wild type mice immunized with OSM using AbISCO-100 and ODN-2395 adjuvants yielded the most number of animals with high antibody titers. Similar results were obtained when serum harvested at day 66 was tested. Interestingly; however, more deaths occurred in groups immunized using AbISCO-100 and ODN-2395 adjuvants, indicating some toxicity at the doses used (see Table below).

TABLE 4
Comparison of immunizations at day 42 and 66

| | Day 42 | | Day 66 | |
|---|---|---|---|---|
| | # of mice with detectable | | # of mice with detectable | |
| | levels of antibody in | # of | levels of antibody in | # of |
| | samples diluted 1:12,500 or | dead | samples diluted 1:12,500 or | dead |
| Group | greater | mice | greater | mice |
| 1 | 4 | 0 | 3 | 1 |
| 2 | 4 | 0 | 6 | 2 |
| 3 | 1 | 0 | 0 | 0 |
| 4 | 5 | 0 | 8 | 1 |
| 5 | 1 | 0 | 2 | 0 |
| 6 | 5 | 0 | 5 | 1 |
| 7 | 1 | 0 | 1 | 0 |
| 8 | 10 | 0 | 7 | 3 |

On day 78 of the study, mice numbers 3074, 3096, 4402, 4418, 4421, 3296 and 4414 were subjected to an additional immunization of antigen with AbISCO-100. Of these mice, numbers 3296 and 4414 received OSM antigen (10 μg/mouse), while the others received PSM as antigen (10 μg/mouse). On day 92 of the study, these mice were bled and subjected to another immunization comprising antigen only. On day 85 of the study, mouse number 4406 was immunized with OSM antigen (100 μg, no adjuvant) and processed for hybridoma formation on day 88.

### Example 2. Synthesis of Glycan Probes

Polyacrylamide (PAA)-conjugated, human serum albumin (HAS)-conjugated or amine-conjugated glycoconjugates are utilized for glycan probe preparation. Glycoconjugates are obtained commercially (e.g. from GlycoTech, Gaithersburg, MD) or are synthesized chemoenzymatically according to the methods described in Yu, H. et al., 2007. Org Biomol Chem. 5:2458-63, the contents of which are herein incorporated by reference in their entirety. Sialoglycans are synthesized using the “one-pot three-enzyme” approach as described by Yu et al (Yu, H. et al., Nat Protoc. 2006. 1(5): 2485-92, Yu, H. et al., J Am Chem Soc. 2005. 127:17618-9 and Yu, H. et al., 2006. Angew Chem Int Ed Engl. 45:3938-44, the contents of each of which are herein incorporated by reference in their entirety).

### Example 3. Sialoglycan-Microarray Production

Arrays are printed on epoxide-derivatized slides (Arrayit Corp, Sunnyvale, CA) with a NanoPrint Microarrayer equipped with 946MP3 Microarray Printing Pins (Arrayit Corporation). Printing is carried out using printing buffers that contain glycans to be printed on the array.

### Example 4. Determination of Optimal Glycan Probe Concentration and Printing Conditions

Various glycan concentrations (6.25, 12.5, 25.0, 50.0, 100.0, 125.0, 150.0, 200.0 and 250.0 μM) and number of replicates (3-6 replicates) are used in 5 different microarray versions to determine the optimal printing and hybridization conditions. The use of a single glycan concentration per probe with 4 replicates/block allows for more blocks per substrate.

Various array printing buffer conditions are examined where changes in 300 mM sodium phosphate buffer pH (7.4-8.4) is used in several different microarray versions to determine the optimal printing and hybridization conditions.

### Example 5. Glycan Array Analysis

Optimized glycan arrays comprise 71 chemically synthesized and well-defined glycans, most of which comprise Neu5Ac and Neu5Gc glycan pairs. Array slides are obtained commercially (ArrayIt Corp, Sunnyvale, CA) and include the glycans listed in the Table below.

TABLE 5
Array glycans

| Glycan | |
|---|---|
| ID No. | Glycan |
| 1 | Neu5,9Ac2α2,3Galβ1,4GlcNAcβO(CH2)2CH2NH2 |
| 2 | Neu5Gc9Acα2,3Galβ1,4GlcNAcβO(CH2)2CH2NH2 |
| 3 | Neu5,9Ac2α2,6Galβ1,4GlcNAcβO(CH2)2CH2NH2 |
| 4 | Neu5Gc9Acα2,6Galβ1,4GlcNAcβO(CH2)2CH2NH2 |
| 5 | Neu5Acα2,6GalNAcαO(CH2)2CH2NH2 |
| 6 | Neu5Gcα2,6GalNAcαO(CH2)2CH2NH2 |
| 7 | Neu5,9Ac2α2,3Galβ1,3GlcNAcβO(CH2)2CH2NH2 |
| 8 | Neu5Gc9Acα2,3Galβ1,3GlcNAcβO(CH2)2CH2NH2 |
| 9 | Neu5,9Ac2α2,3Galβ1,3GalNAcαO(CH2)2CH2NH2 |
| 10 | Neu5Gc9Acα2,3Galβ1,3GalNAcαO(CH2)2CH2NH2 |
| 11 | Neu5Acα2,3Galβ1,4GlcNAcβO(CH2)2CH2NH2 |
| 12 | Neu5Gcα2,3Galβ1,4GlcNAcβO(CH2)2CH2NH2 |
| 13 | Neu5Acα2,3Galβ1,3GlcNAcβO(CH2)2CH2NH2 |
| 14 | Neu5Gcα2,3Galβ1,3GlcNAcβO(CH2)2CH2NH2 |
| 15 | Neu5Acα2,3Galβ1,3GalNAcαO(CH2)2CH2NH2 |
| 16 | Neu5Gcα2,3Galβ1,3GalNAcαO(CH2)2CH2NH2 |
| 17 | Neu5Acα2,6Galβ1,4GlcNAcβO(CH2)2CH2NH2 |
| 18 | Neu5Gcα2,6Galβ1,4GlcNAcβO(CH2)2CH2NH2 |
| 19 | Neu5Acα2,6Galβ1,4GlcβO(CH2)2CH2NH2 |
| 20 | Neu5Gcα2,6Galβ1,4GlcβO(CH2)2CH2NH2 |
| 21 | Neu5Acα2,3Galβ1,4GlcβO(CH2)2CH2NH2 |
| 22 | Neu5Gcα2,3Galβ1,4GlcβO(CH2)2CH2NH2 |
| 23 | Neu5,9Ac2α2,6GalNAcαO(CH2)2CH2NH2 |
| 24 | Neu5Gc9Acα2,6GalNAcαO(CH2)2CH2NH2 |
| 25 | Neu5Acα2,3GalβO(CH2)2CH2NH2 |
| 26 | Neu5Gcα2,3GalβO(CH2)2CH2NH2 |
| 27 | Neu5Acα2,6GalβO(CH2)2CH2NH2 |
| 28 | Neu5Gcα2,6GalβO(CH2)2CH2NH2 |
| 29 | Neu5,9Ac2α2,3GalβO(CH2)2CH2NH2 |
| 30 | Neu5Gc9Acα2,3GalβO(CH2)2CH2NH2 |
| 31 | Neu5,9Ac2α2,6GalβO(CH2)2CH2NH2 |
| 32 | Neu5Gc9Acα2,6GalβO(CH2)2CH2NH2 |
| 33 | Neu5Acα2,3Galβ1,3GalNAcβO(CH2)2CH2NH2 |
| 34 | Neu5Gcα2,3Galβ1,3GalNAcβO(CH2)2CH2NH2 |
| 35 | Neu5,9Ac2α2,3Galβ1,3GalNAcβO(CH2)2CH2NH2 |
| 36 | Neu5Gc9Acα2,3Galβ1,3GalNAcβO(CH2)2CH2NH2 |
| 37 | Neu5,9Ac2α2,6Galβ1,4GlcβO(CH2)2CH2NH2 |
| 38 | Neu5Gc9Acα2,6Galβ1,4GlcβO(CH2)2CH2NH2 |
| 39 | Neu5,9Ac2α2,3Galβ1,4GlcβO(CH2)2CH2NH2 |
| 40 | Neu5Gc9Acα2,3Galβ1,4GlcβO(CH2)2CH2NH2 |
| 41 | Neu5Acα2,8Neu5Acα2,3Galβ1,4GlcβO(CH2)2CH2NH2 |
| 42 | Neu5Acα2,8Neu5Acα2,8Neu5Acα2,3Galβ1,4GlcβO(CH2)2CH2NH2 |
| 43 | Galβ1,4GlcβO(CH2)2CH2NH2 |
| 45 | Galβ1,4GlcNAcβO(CH2)2CH2NH2 |
| 47 | GalNAcαO(CH2)2CH2NH2 |
| 51 | Galβ1,3GalNAcβO(CH2)2CH2NH2 |
| 52 | Galβ1,3GlcNAcαO(CH2)2CH2NH2 |
| 53 | Galβ1,3GlcNAcβO(CH2)2CH2NH2 |
| 54 | Galβ1,4GlcNAc6SβO(CH2)2CH2NH2 |
| 55 | Neu5Acα2,3Galβ1,4(Fucα1,3)GlcNAcβO(CH2)2CH2NH2 |
| 56 | Neu5Gcα2,3Galβ1,4(Fucα1,3)GlcNAcβO(CH2)2CH2NH2 |
| 57 | Neu5Acα2,3Galβ1,4(Fucα1,3)GlcNAc6SβO(CH2)2CH2NH2 |
| 58 | Neu5Gcα2,3Galβ1,4(Fucα1,3)GlcNAc6SβO(CH2)2CH2NH2 |
| 59 | Galβ1,3GlcNAcp1,3Galβ1,4GlcβO(CH2)2CH2NH2 |
| 60 | Neu5Acα2,3Galβ1,3GlcNAcβ1,3Galβ1,4GlcβO(CH2)2CH2NH2 |
| 61 | Neu5Gcα2,3Galβ1,3GlcNAcβ1,3Galβ1,4GlcβO(CH2)2CH2NH2 |
| 62 | Neu5Acα2,3Galβ1,4GlcNAc6SβO(CH2)2CH2NH2 |
| 63 | Neu5Gcα2,3Galβ1,4GlcNAc6SβO(CH2)2CH2NH2 |
| 64 | Neu5Acα2,8Neu5Acα2,3Galβ1,4GlcβO(CH2)3NHCOCH2(OCH2CH2)6NH2 |
| 65 | Neu5Acα2,8Neu5Acα2,8Neu5Acα2,3Galβ1,4GlcβO(CH2)3NHCOCH2(OCH2CH2)6NH2 |
| 66 | Neu5Acα2,6(Neu5Acα2,3)Galβ1,4GlcβO(CH2)2CH2NH2 |
| 67 | Neu5Acα2,6(Neu5Gcα2,3)Galβ1,4GlcβO(CH2)2CH2NH2 |
| 68 | Neu5Acα2,6(KDNα2,3)Galβ1,4GlcβO(CH2)2CH2NH2 |
| 69 | Neu5Gcα2,8Neu5Acα2,3Galβ1,4GlcβO(CH2)2CH2NH2 |
| 70 | KDNα2,8Neu5Acα2,3Galβ1,4GlcβO(CH2)2CH2NH2 |
| 71 | Neu5Acα2,8Kdnα2,6Galβ1,4GlcβO(CH2)2CH2NH2 |
| 72 | Neu5Acα2,8Neu5Gcα2,3Galβ1,4GlcβO(CH2)2CH2NH2 |
| 73 | Neu5Acα2,8Neu5Gcα2,6Galβ1,4GlcβO(CH2)2CH2NH2 |
| 74 | KDNα2,8Neu5Gcα2,3Galβ1,4GlcβO(CH2)2CH2NH2 |
| 75 | Neu5Gcα2,8Neu5Gcα2,3Galβ1,4GlcβO(CH2)2CH2NH2 |
| 76 | Neu5Acα2,8Neu5Acα2,6Galβ1,4GlcβO(CH2)2CH2NH2 |

300 ml of epoxy blocking buffer is prepared by combining 15 ml of 2 M Tris buffer (pH 8) with 0.9 ml of 16.6 M ethanolamine and 284.1 ml of distilled water. The solution is adjusted to pH 9.0 and then filtered using a 0.2 μM nitrocellulose membrane. The epoxy buffer solution as well as 1 L of distilled water are pre-warmed to 50° C. Glass slides are arranged in a slide holder and quickly submerged in a staining tub with the warmed epoxy blocking buffer. Slides are incubated in the epoxy blocking buffer for 1 hour at 50° C. with periodic shaking to deactivate epoxy binding sites. Next, slides are rinsed and then blocked with PBS with 1% OVA at 25° C. for one hour. Serum samples (diluted 1:1000) or purified antibodies/lectins (0.5-40ug/mL) are diluted in PBS with 1% OVA and added to the glycan array for one hour at 25° C. After extensive washing, binding of primary agents is detected by incubating glycan microarray slides with Cy3-conjugated secondary antibody (Jackson Immunoresearch, West Grove, PA) for one hour. Slides are then washed extensively, dried and scanned with a Genepix 4000B scanner (Laser at 100%; gain at 350; 10 m pixels). Raw data from scanned images are extracted using the Genepix software and analysis of raw data is carried out. Sera/antibodies/lectins are considered to be highly specific for AcSTn and GcSTn if they demonstrate binding to both molecules, but not to Tn or any other glycans on the array. Linear regression was used to determine preferential antibody binding with and without adjustments for experiment-to-experiment variation. On smaller sample sets, a two-sided Wilcoxon rank sum test was used to determine preferential binding.

### Example 6. Flow Cytometry-Based Analysis of Antibody Binding

Flow cytometry-based analysis is carried out to elucidate the curve-dose response for binding of antibodies to cell surface antigens. For these analyses, three cell lines are employed.

MDA-MB-231 cells are human breast cancer cells. They are grown in Earle's Minimum Essential Medium supplemented with 10% fetal calf serum (FCS), 100 μg/ml penicillin, 100 UI/ml streptomycin and 45 μg/ml gentamycin. MCF-7 cells are also human breast cancer cells and are grown under the same conditions as MDA-MB-231 cells. Stably transfected versions of MDA-MB-231 and MCF-7 cells (clone TAH3.P10 for MDA-MB-231 cells and clone A12.1 for MCF-7 cells) that over express GalNAc α2,6-sialyltransferase (ST6GalNAc 1,) are also cultured under the same conditions with the exception of an added 1 mg/ml of G418 to support cells expressing the transgene. ST6GalNAc 1 is an enzyme capable of sialylating GalNAc. As a result of over expression, transfected cells express high levels of Neu5Ac-STn (see Julien, S. et al., Glycoconjugate journal. 2001. 18, 883-93; the contents of which are herein incorporated by reference in their entirety). E3 cells are murine breast cancer cells. They are cultured in Dulbecco's E4 medium with 10% FCS. Stably transfected versions of E3 cells expressing high levels of Neu5Gc-STn (E3-STn) are cultured with 600 μg/ml of G418 and 200 μg/ml hygromycin. During growth and maintenance of experimental cells, trypsin is not used for cell passaging.

For analysis, cells are harvested using 10 mM EDTA and washed with PBS comprising 1% BSA before pelleting by light centrifugation. Cell numbers and viability are determined by trypan blue dye exclusion analysis and cell concentrations are adjusted to 5×10⁶ cells/ml in PBS with 1% BSA. 50 μl of cells are added to each well of an assay plate. Cells are combined with 50 μl solutions of antibody being analyzed or control antibodies and incubated for 1 hour at 4° C. Cells are washed and pelleted twice with PBS with 1% BSA before being treated with 100 μl of PBS with 1% BSA comprising a 1:1,500 dilution of anti-mouse IgG (Southern Biotech, Birmingham, Alabama,) conjugated to allophycocyanin (APC). Cells are incubated for 30 min at 4° C. before washing and resuspending in 200 μl of propidium iodide (PI) diluted 1:1000 in PBS with 1% BSA. Treated cells are then subjects to flow cytometry analysis and 10,000 events are acquired for each sample.

### Example 7. Flow Cytometry Analysis of Antibody Internalization

Flow cytometry analysis is carried out in order to quantify the extent of antibody internalization according to the procedure of Example 6, with several notable distinctions.

For analysis, stably transfected variants of MDA-MB-231 cells (clone TAH3.P10) that express high levels of cell surface-bound Neu5Ac-STn are harvested using 10 mM EDTA and washed with PBS comprising 1% BSA before pelleting by light centrifugation. Cell numbers and viability are determined by trypan blue dye exclusion analysis and cell concentrations are adjusted to 5×10⁶ cells/ml in PBS with 1% BSA. 50 μl of cells are added to each well of an assay plate. Cells are combined with 50 μl solutions of antibody or fluorescently-labeled antibody and incubated for 1 hour at 4° C. Following this incubation period, cells are washed with PBS to remove unbound antibody and aliquots are removed for incubation for various times (15, 30, 60 minutes) at 37° C. to allow bound antibody to internalize at a physiologically relevant temperature. After each incubation, cell surface-bound antibody is removed by treating cells with acidic medium (150 mM NaCl, pH=2.5) Cells treated with unlabeled antibody are washed with PBS and fixed with paraformaldehyde fixation buffer (PFA) containing 3% paraformaldehyde and 2% sucrose in PBS for 15 minutes at room temperature. These cells are rinsed again in PBS and treated with blocking buffer made up of PBS with 1% bovine serum albumin (BSA). Cells are incubated for 30 min at room temperature, rinsed in PBS and treated with secondary antibody (allophycocyanin-labeled goat-anti-mouse IgG) for 2 hours at room temperature. All cells are then washed with PBS and subjected to flow cytometry analysis wherein 10,000 events are recorded for each sample. Residual fluorescent signal in acid-treated samples is further quenched via treatment with trypan blue dye.

### Example 8. Evaluate Antibody Internalization Through Cell Viability Assay

Cell viability assays are performed to screen anti-STn antibodies of the present invention in the presence and absence of secondary antibody-drug conjugates (2° ADCs). The purpose of the screen is to identify the ability of each anti-STn antibody to inhibit cell growth. Antibodies with potent cell growth inhibition are used to design direct antibody-drug conjugates (ADCs). Using such secondary antibody-drug conjugates (2° ADCs) in cell-based cytotoxic assays can quickly pre-screen many ADC candidates against tumor cells. Based on the assay, a naked antibody candidate is directly added to cells in the presence of a 2° ADC. Internalization of the mAb/2° ADC complex into cells that express a high density of the targeted antigen can achieve a dose-dependent drug release within the cells, causing a cytotoxic effect to kill the cells (e.g., tumor cells), while cells expressing a low density of the targeted antigen are not affected (e.g., normal cells).

To perform cell viability assays, cell lines described in the present application (MDA-MB-231 parental, MDA-MB-231-STn+, and OV-90) are prepared and cultured for the assays. The cell culture is optimized for cell density by plating different densities of cells (e.g., 2,000, 4,000 and 7,500 per well) on a 96-well plate and observing the cell growth for 96 hours. The plating condition in which cells reach around 90% confluence at the end of the 96 hours is identified and the optimal cell number is then used in the final viability assay.

Antibodies are tested in one or more cell lines in the presence and absence of a 2° ADC such as Fab αMFc-CL-MMAF. Duplicate or triplicate cell plates for each cell line are used for testing each antibody candidate.

For cell viability assays, data points are collected for each antibody candidate with duplicates for each data point. Each antibody candidate is diluted in serial concentrations from 0.3 pM to 20 nM. A constant amount of Fab αMFc-CL-MMAF (40 nM) is used in the viability assay.

Alternatively, data points are collected for each antibody candidate with triplicates for each data point. Each antibody candidate is diluted in serial concentrations from 1 μM to 20 nM. A constant amount of Fab αMFc-CL-MMAF (40 nM) is used in the viability assay.

Cell viabilities are measured by Cell-Titer Glo luminescence based assays.

### Example 9. Phage Library Construction and Selection

RNA is prepared from spleens harvested from mice with a strong immune response to immunization. Mouse variable (V) regions are PCR amplified and assembled into scFv expression constructs. ScFv sequences are cloned into phagemid display vectors allowing for scFv display on the surface of M13 phage particles. The resulting library is transformed into E. coli (TG1). Bulk transformations of E. coli are grown and phage are prepared by phage rescue. In the first round of selection, phage from the culture medium are purified by PEG precipitation.

Candidate scFvs are selected using both negative and positive selection methods. For negative selection, the library is incubated with “destroyed” STn-negative mucin (e.g. chemically treated PSM). For positive selection, the library is incubated with GcSTn mucin (e.g. PSM and/or de-O-acetylated BSM), AcSTn mucin (e.g. OSM and/or de-O-acetylated BSM) or BSM (and/or de-O-acetylated BSM) and a synthetic glycan (Neu5Gc and/or Neu5Ac) in the presence of a Neu5Ac or Neu5Gc (depending on the desired target).

After 3-4 rounds of selection with reducing antigen concentrations, 1000 clones are analyzed by ELISA for binding to STn (e.g. Neu5Ac and/or Neu5Gc) using synthetic and natural glycan targets. Lead phage/scFv candidates are tested in a secondary flow cytometry-based cellular assay for binding to GcSTn and/or AcSTn using Jurkat cells with or without “induction” of GcSTn or AcSTn. Up to 20 selected scFv candidates of interest are subjected to further analysis.

Lead scFv candidates are selected for conversion to IgG. Variable regions from each scFv are cloned into mammalian expression vectors between an upstream CMV promoter and a downstream immunoglobulin constant region. Heavy chain vector includes murine IgG1 and κ constant regions. Vectors are transiently transfected into HEK293/EBNA cells. Antibody samples are purified and characterized by binding to positive and negative glycan epitopes. Samples of up to 0.5 mg of each whole IgG are further analyzed.

### Example 10. Antibody-Dependent Cell-Mediated Cytotoxicity Optimization

Genes encoding the variable regions of a selected IgG are cloned into mammalian expression vectors encoding human Fc regions (huIgGlκ) containing amino acid mutations known to enhance Fc-receptor binding and antibody-dependent cell-mediated cytotoxicity (ADCC). Vectors are transiently transfected into HEK293/EBNA cells. After 48 hours, IgG expression is quantified and samples of antibody are purified on protein A columns. Antibodies are then tested in ADCC assays. Neu5Gc and Neu5Ac-expressing Jurkat cell lines are used as the target cells and human peripheral blood mononuclear cells (PBMC) are used as a source of effector cells. Target cells are titrated using maximum cell lysis to determine the optimum cell density for use in multiwall plate format assay. ADCC-mutated antibody together with the non-mutated IgG are pre-incubated with target cells, effector cells are then added at varying target:effector cell ratios, and cultures are incubated at 37° C. Percentage viability is determined using Calcein-AM dye (BD Biosciences, San Jose, CA) release. Samples of up to 0.5 mg of ADCC-mutated IgG are subjected to further analysis.

### Example 11. Production of Lead Antibody from Semi-Stable HEK Cell Line

Variable regions from IgG are cloned into mammalian expression vectors between an upstream CMV promoter and a downstream immunoglobulin constant region. Heavy chain vector includes murine IgG1 and κ constant regions. Vectors are transiently transfected into HEK293/EBNA cells and antibody titers are assessed at 72 hours. Transiently transfected HEK293/EBNA cells are selected with hygromycin to establish a semi-stable expression system. Semi-stable cells are expanded to 10 liters. Antibodies are purified from the culture supernatant by Protein A, dialyzed into PBS and the resulting preparation is analyzed for (1) aggregates by analytical size exclusion chromatography (SEC), (2) endotoxin levels by Limulus amebocyte lysate (LAL) testing (expressed as EU/mg), and (3) binding to antigen in the primary assay.

### Example 12. Additional Assays for Screening scFv Candidates for Target Affinity

ScFv candidates are subjected to additional screening methods for STn (pan-STn, AcSTn and/or GcSTn) affinity using a variety of proposed targets.

### Synthetic Glycan Target Screening

As used herein, the term “target screening” refers to the use of a target substance to identify binding partners for that substance. Synthetic glycan target screening is carried out using desired STn target antigens bound to poly(acrylic acid) (PAA) with a biotin tag. Undesired STn target antigens as well as Tn bound to PAA with a biotin tag are used as negative controls. Cells associated with candidate scFvs are isolated through precipitation with avidin-associated entities.

### Natural Glycan Target Screening on Live Cells

Target screening using live cells is carried out using Jurkat cells fed with sialic acid (Neu5Gc and/or Neu5Ac, depending on the desired antibody target) or Jurkat cells fed with an alternative form of sialic acid (Neu5Gc and/or Neu5Ac, depending on the desired antibody target) as a negative control. Target screening using live cells is also carried out using MCF-7 or MDA-MB-231 cells fed with sialic acid (Neu5Gc and/or Neu5Ac, depending on the desired antibody target or whether being used for negative control screening) and stable transfection. Flow cytometry is used in either case to isolate cells associated with scFv candidates.

### Natural Glycan Target Screening on Tissue (Ex Vivo)

Target screening using ex vivo tissue is carried out using biopsy tissue samples. Binding of scFv candidates with ex vivo tissue is analyzed using standard immunohistochemical methods. Single tissue sections as well as tissue microarray sections are used. Samples are treated with or without sialidase and/or periodate in control experiments.

### Example 13. Antibody Humanization

Fully humanized heavy and light chains are designed. Protein models of the variable regions are generated using existing antibody structures as templates. Segments of starting heavy and light chain variable region amino acid sequences are compared with human sequences for possible inclusion in the fully humanized sequences. Series of humanized heavy and light chain variable regions are designed entirely from segments of human variable region sequences with the objective that T cell epitopes be avoided. Variant human sequence segments with significant incidence of potential T cell epitopes as determined by in silico technologies are discarded.

Humanized heavy and light chain variable region genes are constructed from overlapping oligonucleotides assembled into full length genes using the ligase chain reaction (LCR). LCR products are amplified and suitable restriction sites are added for cloning into expression vectors. PCR products are cloned into intermediate vectors and confirmed by sequencing.

For construction of expression plasmids encoding fully humanized antibodies with human constant regions, DNA sequences for each variable region are inserted into mammalian expression vectors between an upstream cytomegalovirus immediate/early promoter/enhancer (CMV IE) plus the immunoglobulin signal sequence and a downstream immunoglobulin constant region gene. DNA samples are prepared for transfection into mammalian cells.

For generation of cell lines and selection of lead fully humanized antibodies, heavy and light chain plasmid DNA pairs are transfected into mammalian cells (NS0). Cell lines producing humanized antibodies are expanded and antibody samples are purified. Antibodies are tested in primary and secondary binding assays to determine leading antibody candidates. The 3 leading candidates are used for further analysis.

### Example 14. Immunogenicity Testing

Lead antibodies are subjected to EpiScreen (Antitope, Paradise Valley, AZ) whole antibody human T cell assays using a minimum of 20 blood samples from healthy volunteer donors. Immunogenicity of lead antibodies is compared with control chimeric antibodies with starting antibody variable regions and matched human constant regions. Data are benchmarked against EpiScreen whole protein data for clinical-stage biologics.

### Example 15. Cell Line Development

Cell lines are developed with the ability to yield high levels of antibody with no non-human glycosylation due to knock down of the CMAH gene. Cell lines are glycoengineered to increase ADCC. These cell lines have the ability to perform in small and large scale production.

### Example 16. Glycan Array

A glycan array is constructed by attaching at least four glycans to a substrate by a linker.

### Example 17. Sialoglycan Array

A glycan array is constructed by attaching at least four glycans to a substrate. Glycans are selected such that the final array is made up of glycans having at least one sialic acid residue. The glycans are further selected such that the final array is made up of 50% glycans with Neu5Ac and 50% glycans with Neu5Gc.

### Example 18. Paired Sialoglycan Array

A glycan array is constructed by attaching at least four glycans to a substrate. Glycans are selected such that the final array is made up of glycans having at least one sialic acid residue. The glycans are further selected such that the final array is made up of glycan pairs that differ only by the presence of Neu5Ac on one glycan of each pair and Neu5Gc on the other glycan of each pair.

### Example 19. Large Paired Sialoglycan Array

A glycan array is constructed by attaching at least 40 glycan pairs, each pair differing by the substitution of a Neu5Gc residue for a Neu5Ac residue.

### Example 20. ELISA Analysis

96-well plates are coated with one or more glycans and incubated overnight at 4° C. Wells are then blocked with PBS with 1% albumin. Samples to be analyzed are serially diluted in PBS with 1% albumin. Samples, as well as negative and positive control samples, are added to individual wells and specific binding of entities in the samples to the bound glycans is determined using horseradish peroxidase (HRP)-conjugated antibodies capable of binding the entities. Bound HRP-conjugated antibodies are detected by incubating the wells with an HRP substrate. The reaction is stopped by addition of sulfuric acid. Optical densities (ODs) are measured at 490 nm. The titer of entities in the samples is obtained by comparison of OD values with a cutoff value calculated as two standard deviations above the mean of the OD values of the negative control sample. Sample tests are considered positive if the mean optical density value is greater than the cutoff value.

### Example 21. Anti-STn Animal Serum Titer Determination and Mouse Selection

Anti-STn serum titer is determined using a murine anti-STn bovine submaxillary mucin (BSM) ELISA together with serum profiles observed by glycan microarray. 96-well plates are coated with 1 μg/well of BSM and incubated overnight at 4° C. O-acetylation of BSM antigen is removed by treating wells with 0.1 M sodium hydroxide. Specific binding to STn is determined by treatment of wells with sodium periodate. Periodate treatment destroys the C6 side chain of sialic acid; therefore antibodies raised against STn should not bind to periodate-treated wells. Wells are blocked with PBS 1% ovalbumin (OVA). Serum samples to be assayed are serially diluted in PBS 1% OVA. A commercially available mouse anti-STn monoclonal antibody, 3F1 (SBH Sciences, Natick, MA) is used as a positive control. This antibody is also serially diluted in PBS with 1% OVA. A pool of serum from naïve wild type mice is used for the preparation of negative control samples. Detection of bound anti-STn antibodies is determined using an HRP-conjugated polyclonal goat anti-mouse IgG antibody (Jackson Immunoresearch, West Grove, PA). HRP-conjugated antibodies are added and incubated for one hour at room temperature. Wells are rinsed, followed by treatment with a substrate for HRP for 30 minutes. The reaction is stopped by addition of sulfuric acid (1.6 M). Optical densities are measured at 490 nm using a Spectramax microplate reader (Molecular Devices, Sunnyvale, CA). The serum titer is obtained by comparison of OD values with a cutoff value calculated as two standard deviations above the mean of optical density values of the negative control. Sample tests are considered positive if the mean optical density value is greater than the cutoff value.

### Example 22. Anti-Glycan Antibody Profile

A subject sample is obtained and an anti-sialoglycan antibody profile is generated for the sample. The antibody profile consists of results from sialoglycan array analysis. The sample is diluted and incubated with a sialoglycan array. The sialoglycan array comprises chemically synthesized and well-defined glycan pairs attached to an array slide. Each pair includes a glycan comprising Neu5Ac and a glycan comprising Neu5Gc.

300 ml of epoxy blocking buffer is prepared by combining 15 ml of 2 M Tris buffer (pH 8) with 0.9 ml of 16.6 M ethanolamine and 284.1 ml of distilled water. The solution is filtered using a 0.2 μM nitrocellulose membrane. The epoxy buffer solution as well as 1 L of distilled water are pre-warmed to 50° C. Glass slides are arranged in a slide holder and quickly submerged in a staining tub with the warmed epoxy blocking buffer. Slides are incubated in the epoxy blocking buffer for 1 hour at 50° C. with periodic shaking to deactivate epoxy binding sites. Next, slides are rinsed and blocked with PBS with 1% OVA at 25° C. for one hour. Samples are diluted in PBS with 1% OVA and added to the glycan array for one hour at 25° C. After extensive washing, binding of sample antibodies are detected by incubating sialoglycan microarray slides with Cy3-conjugated anti-mouse IgG (Jackson Immunoresearch, West Grove, PA) for one hour. Slides are then washed extensively, dried and scanned with a Genepix 4000B scanner (Laser at 100%; gain at 350; m pixels). Raw data from scanned images are extracted using the Genepix software and analysis of raw data is carried out.

Results indicate the presence of antibodies in the sample that are capable of binding to glycan probes in the array.

### Example 23. Expanded Anti-Glycan Antibody Profile

The anti-glycan antibody profile obtained in the previous example is expanded through the use of a binding assay to produce an anti-glycan antibody profile with additional information. To generate the additional information, samples are subjected to ELISA analysis. The anti-glycan antibody profile is updated based on ELISA analysis results.

### Example 24. Tumor Glycan Profile

An anti-TACA antibody array is prepared by linking a panel of anti-TACA antibodies to an array substrate. Tumor tissue being subjected to glycan profiling is solubilized and resulting samples are incubated with the anti-TACA antibody array. Binding of antigens present in the tumor samples to spots on the anti-TACA antibody array are detected using surface plasmon resonance techniques. A tumor glycan profile is generated from the results.

### Example 25. Altering pH in Array Printing Buffer

The pH of standard printing buffer with 100 μM glycans was lowered from 8.4 to a more neutral pH of 7.4 to keep 9-O acetyl groups intact on sialic acids. Glycan arrays were printed with standard or the neutral pH printing buffer and anti-STn antibody, 3F1 (SBH Biosciences, Natick, MA) was used to test printed arrays (see the following Table).

TABLE 6
Array results

| Glycan | | Fluorescence | Fluorescence |
|---|---|---|---|
| ID | | intensity (pH | intensity (pH |
| No. | Glycan Structure | 7.4 buffer) | 7.4 buffer) |
| 5 | Neu5Acα6GalNAcαO(CH2)2CH2NH2 | 303 | 4673 |
| 6 | Neu5Gcα6GalNAcαO(CH2)2CH2NH2 | 212 | 1831 |
| 23 | Neu5,9Ac2α6GalNAcαO(CH2)2CH2NH2 | 192 | 2668 |
| 24 | Neu5Gc9Acα6GalNAcαO(CH2)2CH2NH2 | 123 | 1353 |

Arrays printed with the more neutral pH buffer altered the binding profile of 3F1 observed with standard printing buffer. Arrays printed with the more neutral printing buffer yielded about a ten-fold loss in fluorescence intensity signal for glycans Neu5Ac-STn (glycan ID number 5), Neu5Gc-STn (glycan ID number 6), Neu5,9Ac2-STn (glycan ID number 23), and Neu5Gc9Ac-STn (glycan ID number 24) when probed with 3F1.

### Example 26. Altering Glycan Concentration in Printing Buffer

Printing buffers were prepared with varying concentrations of glycans to generate glycan arrays with altered glycan density. These printing buffers included 50 μM, 100 μM (which is the standard concentration used), and 200 μM glycan concentrations. Arrays were printed with each printing buffer and anti-STn antibody, 3F1 (SBH Biosciences, Natick, MA) was used to test printed arrays (see the following Table).

TABLE 7
Array results

| Glycan | Fluorescence | Fluorescence | Fluorescence |
|---|---|---|---|
| ID | intensity (50 μM | intensity (100 μM | intensity (200 μM |
| No. | glycans) | glycans) | glycans) |
| 5 | 317 | 4312 | 197 |
| 6 | 60 | 1542 | 83 |
| 23 | 148 | 3449 | 58 |
| 24 | 74 | 2351 | 78 |

Changes in printing buffer glycan concentration altered the 3F1 antibody binding profile. 3F1 binding to arrays printed with lower (50 μM) or higher (200 μM) glycan concentrations yielded fluorescence intensity signals that were 20-30 fold less than with arrays printed with standard (100 μM) glycan concentrations.

1. A glycan array comprising:
a. a substrate, and
b. at least four glycans, each attached to said substrate by a linker, wherein the percentage of attached glycans comprising N-acetylneuraminic acid (Neu5Ac) is from 25% to 75%.

2. The glycan array of claim 1, wherein said at least four glycans are independently selected from the group consisting of:
Araα1,2Araα-R;
Araα1,2Glcβ-R;
Araα1,3Glcβ-R;
Araα1,4Glcβ-R;
Araα1,5Araα-R;
Araα1,6Glcβ-R;
Fucα1,2[Galβ1,4]GlcNAcα-R;
Fucα1,2[Galβ1,4]GlcNAcβ-R;
Fucα1,2[Galβ1,4]GlcNAcβ-R;
Fucα1,2[Galβ1,4]Glcβ-R;
Fucα1,2Galβ1,3GlcNAcβ1,3Galβ-R;
Fucα1,2Galβ1,3GlcNAcβ-R;
Fucα1,2Galβ1,4[Fucα1,3]GlcNAcβ-R;
Fucα1,2Galβ1,4GlcNAcβ1,3Galβ-R;
Fucα1,2Galβ1,4GlcNAcβ-R;
Fucα1,2Galβ-R;
Fucα1,3[Fucα1,2Galβ1,4]GlcNAcβ-R;
Fucα1,3[Galβ1,4]GlcNAcβ1,3Galβ-R;
Fucα1,3[Galβ1,4]GlcNAcβ1,6Galβ-R;
Fucα1,3[Galβ1,4]GlcNAcβ-R;
Fucα1,3[GlcNAcβ1,3Galβ1,4]GlcNAcβ-R;
Fucα1,3GlcNAcβ1,3Galβ1,4Glcβ-R;
Fucα1,3GlcNAcβ1,3Galβ-R;
Fucα1,3GlcNAcβ1,6[GlcNAcβ1,3]Galβ-R;
Fucα1,3GlcNAcβ1,6Galβ-R;
Fucα1,3GlcNAcβ1,6Galβ1,4Glcβ-R;
Fucα1,3GlcNAcβ-R;
Fucα1,3Glcβ-R;
Fucα1,4[Galα1,3]GlcNAcβ1,3Galβ-R;
Fucα1,4[Galβ1,3]GlcNAcβ1,3Galβ-R;
Fucα1,4[Galβ1,3]GlcNAcβ-R;
Fucα1,4GlcNAcβ1,3Galβ1,4Glcβ-R;
Fucα1,4GlcNAcβ1,3Galβ-R;
Fucα1,4GlcNAcβ-R;
Fucα1,6[GlcNAcβ1,4]Manα-R;
Fucα1,6[Manβ1,4GlcNAcβ1,4]GlcNAcβ-R;
Fucα1,6GlcNAcβ-R;
Fucβ1,4GlcNAcβ1,3Galβ-R;
GalNAcα1,3[Fucα1,2]Galβ1,4-R;
GalNAcα1,3[Fucα1,2]Galβ-R;
GalNAcα-R;
GalNAcβ1,3Galβ1,4Galβ1,4Glcβ-R;
GalNAcβ1,4[Neu5Acα2,3]Galβ1,4GlcNAcβ-R;
GalNAcβ1,4Galβ1,4Glcβ-R;
Galα1,2Galα-R;
Galα1,3[Fucα1,2]Galβ1,4-R;
Galα1,3Galα-R;
Galα1,3Galβ1,4GlcNAcβ-R;
Galα1,6Galα-R;
Galβ1,2Galβ-R;
Galβ1,3GalNAcβ-R;
Galβ1,3Galβ1,4Xylβ-R;
Galβ1,3Galβ-R;
Galβ1,3GlcNAcα-R;
Galβ1,3GlcNAcβ1,3Galβ1,4Glcβ-R;
Galβ1,3GlcNAcβ1,3Galβ-R;
Galβ1,3GlcNAcβ1,6Galβ1,4Glcβ-R;
Galβ1,3GlcNAcβ-R;
Galβ1,4[Fucα1,3]GlcNAcβ-R;
Galβ1,4GlcNAc1,4[GlcNAcβ1,2]Manα-R;
Galβ1,4GlcNAc6SB-R;
Galβ1,4GlcNAcβ1,3Galβ1,4GlcNAcβ-R;
Galβ1,4GlcNAcβ1,3Galβ1,4Glcβ-R;
Galβ1,4GlcNAcβ1,3Galβ-R;
Galβ1,4GlcNAcβ1,4[GlcNAcβ1,2]Manα-R;
Galβ1,4GlcNAcβ1,6Galβ-R;
Galβ1,4GlcNAcβ1,6Galβ1,4Glcβ-R;
Galβ1,4GlcNAcβ-R;
Galβ1,4Glcβ-R;
Galβ1,4Xylβ-R;
Galβ1,6Galβ-R;
Galβ1,6Galβ1,4Gal1,4Glcβ-R;
Galβ1,6Galβ1,4Galβ1,4Glcβ-R;
GlcAβ1,3Galβ1,3Gal1,4XylB-R;
GlcAβ1,3Galβ1,3Galβ1,4Xylβ-R;
GlcNAcβ1,2Manα1,3[Manα1,6]Manβ-R;
GlcNAcβ1,3[Galβ1,6]GlcNAcβ-R;
GlcNAcβ1,3[GlcNAcβ1,6]GalNAcβ-R;
GlcNAcβ1,3[GlcNAcβ1,6]Galβ-R;
GlcNAcβ1,30[GlcNAcβ1,6]Galβ-R;
GlcNAcβ1,3GalNAcα-R;
GlcNAcβ1,3GalNAcβ-R;
GlcNAcβ1,3Galα-R;
GlcNAcβ1,3Galβ1,3GalNAcβ-R;
GlcNAcβ1,3Galβ1,4GlcNAcβ1,3Galβ-R;
GlcNAcβ1,3Galβ1,4GlcNAcβ-R;
GlcNAcβ1,3Galβ-R;
GlcNAcβ1,4[Fucα2,6]GlcNAcβ-R;
GlcNAcβ1,4[Galβ1,4GlcNAcβ1,2]Manα-R;
GlcNAcβ1,4[GlcNAcβ1,2]Manα-R;
GlcNAcβ1,4GlcNAcα-R;
GlcNAcβ1,4GlcNAcβ-R;
GlcNAcβ1,6[Galβ1,3]GalNAcβ-R;
GlcNAcβ1,6[Galβ1,3]GlcNAcβ-R;
GlcNAcβ1,6[Galβ1,3GlcNAcβ1,3]Galβ-R;
GlcNAcβ1,6[GlcNAcβ1,3]Galβ1,4Glcβ-R;
GlcNAcβ1,6GalNAcβ1,3Galα-R;
GlcNAcβ1,6Galα-R;
GlcNAcβ1,6Galβ-R;
GlcNAcβ1,6Galβ1,3GlcNAcβ-R;
GlcNAcβ1,6Galβ1,4GlcNAcβ-R;
Glcα1,2Glcα-R;
Glcα1,3Glcα-R;
Glcα1,4Glcα-R;
Glcα1,6Glcα-R;
Glcβ1,2Glcβ-R;
Glcβ1,3Glcβ-R;
Glcβ1,6Gicβ-R;
Glcβ1,6Glcβ-R;
KDNα2,8Neu5Acα2,3Galβ1,4Glcβ-R;
KDNα2,8Neu5Gcα2,3Galβ1,4Glcβ-R;
Manα1,2Manα1,2Manα-R;
Manα1,2Manα-R;
Manα1,3[Manα1,6]Manβ1,4GlcNAcβ-R;
Manα1,3Manα1,2Manα1,2Manα-R;
Manα1,3Manα1,4GlcNAcβ1,4GlcNAcβ-R;
Manα1,3Manα-R;
Manα1,4GlcNAcβ1,4[Fucα1,6]GlcNAcβ-R;
Manα1,4GlcNAcβ1,4GlcNAcβ-R;
Manα1,6Manα-R;
Manα1,6Manα1,4GlcNAcβ1,4GlcNAcβ-R;
Manβ1,4GlcNAcβ1,4[Fucα1,6]GlcNAcβ-R;
Manβ1,4GlcNAcβ1,4[Fucα2,6]GlcNAcβ-R;
Manβ1,4GlcNAcβ1,4GicNAcβ-R;
Manβ1,4GlcNAcβ1,4GlcNAcβ-R;
Manβ1,4GlcNAcβ-R;
Neu5,9Ac2α2,3Galβ1,3GalNAcα-R;
Neu5,9Ac2α2,3Galβ1,3GalNAcβ-R;
Neu5,9Ac2α2,3Galβ1,3GlcNAcβ-R;
Neu5,9Ac2α2,3Galβ1,4GlcNAcβ-R;
Neu5,9Ac2α2,3Galβ1,4Glcβ-R;
Neu5,9Ac2α2,3Galβ-R;
Neu5,9Ac2α2,6GalNAcα-R;
Neu5,9Ac2α2,6Galβ1,4GlcNAcβ-R;
Neu5,9Ac2α2,6Galβ1,4Glcβ-R;
Neu5,9Ac2α2,6Galβ-R;
Neu5Acα2,3Galβ1,3 [Neu5Acα2,6]GalNAcα-R;
Neu5Acα2,3Galβ1,3GalNAcα-R;
Neu5Acα2,3Galβ1,3GalNAcβ-R;
Neu5Acα2,3Galβ1,3GlcNAcα-R;
Neu5Acα2,3Galβ1,3GlcNAcβ1,3Galβ1,4Glcβ-R;
Neu5Acα2,3Galβ1,3GlcNAcβ-R;
Neu5Acα2,3Galβ1,4(Fucα1,3)GlcNAc6SB-R;
Neu5Acα2,3Galβ1,4(Fucα1,3)GlcNAcβ-R;
Neu5Acα2,3Galβ1,4[Fucα1,3]GlcNAcβ-R;
Neu5Acα2,3Galβ1,4GlcNAc6SB-R;
Neu5Acα2,3Galβ1,4GlcNAcα-R;
Neu5Acα2,3Galβ1,4GlcNAcβ-R;
Neu5Acα2,3Galβ1,4Glcβ-R;
Neu5Acα2,3Galβ-R;
Neu5Acα2,6(KDNα2,3)Galβ1,4Glcβ-R;
Neu5Acα2,6(Neu5Acα2,3)Galβ1,4Glcβ-R;
Neu5Acα2,6(Neu5Gcα2,3)Galβ1,4Glcβ-R;
Neu5Acα2,6GalNAcα-R;
Neu5Acα2,6GalNAcα-R;
Neu5Acα2,6Galβ1,3GalNAcα-R;
Neu5Acα2,6Galβ1,4GlcNAcα-R;
Neu5Acα2,6Galβ1,4GlcNAcβ-R;
Neu5Acα2,6Galβ1,4GlcNAcβ-R;
Neu5Acα2,6Galβ1,4Glcβ-R;
Neu5Acα2,6Galβ-R;
Neu5Acα2,8KDNα2,6Galβ1,4Glcβ-R;
Neu5Acα2,8Neu5Acα2,3Galβ1,4Glcβ-R;
Neu5Acα2,8Neu5Acα2,3Galβ-R;
Neu5Acα2,8Neu5Acα2,6Galβ1,4Glcβ-R;
Neu5Acα2,8Neu5Acα2,8Neu5Acα2,3Galβ1,4Glcβ-R;
Neu5Acα2,8Neu5Gcα2,3Galβ1,4Glcβ-R;
Neu5Acα2,8Neu5Gcα2,6Galβ1,4Glcβ-R;
Neu5Gc9Acα2,3Galβ1,3GalNAcα-R;
Neu5Gc9Acα2,3Galβ1,3GalNAcβ-R;
Neu5Gc9Acα2,3Galβ1,3GlcNAcβ-R;
Neu5Gc9Acα2,3Galβ1,4GlcNAcβ-R;
Neu5Gc9Acα2,3Galβ1,4Glcβ-R;
Neu5Gc9Acα2,3Galβ-R;
Neu5Gc9Acα2,6GalNAcα-R;
Neu5Gc9Acα2,6Galβ1,4GlcNAcβ-R;
Neu5Gc9Acα2,6Galβ1,4Glcβ-R;
Neu5Gc9Acα2,6Galβ-R;
Neu5GcOMea2,8Neu5Acα2,3Galβ1,4Glcβ-R;
Neu5Gcα2,3Galβ1,3GalNAcα-R;
Neu5Gcα2,3Galβ1,3GalNAcβ-R;
Neu5Gcα2,3Galβ1,3GlcNAcβ1,3Galβ1,4Glcβ-R;
Neu5Gcα2,3Galβ1,3GlcNAcβ-R;
Neu5Gcα2,3Galβ1,4(Fucα1,3)GlcNAc6Sβ-R;
Neu5Gcα2,3Galβ1,4(Fucα1,3)GlcNAcβ-R;
Neu5Gcα2,3Galβ1,4GlcNAc6Sβ-R;
Neu5Gcα2,3Galβ1,4GlcNAcβ-R;
Neu5Gcα2,3Galβ1,4Glcβ-R;
Neu5Gcα2,3Galβ-R;
Neu5Gcα2,6GalNAcα-R;
Neu5Gcα2,6Galβ1,4GlcNAcβ-R;
Neu5Gcα2,6Galβ1,4Glcβ-R;
Neu5Gcα2,6Galβ-R;
Neu5Gcα2,8Neu5Acα2,3Galβ1,4Glcβ-R;
Neu5Gcα2,8Neu5Gcα2,3Galβ1,4Glcβ-R;
NeuAcα2,3Galβ1,3[NeuAcα2,6]GalNAcα-R;
Xylα1,2Manα-R;
Xylα1,3Glcβ-R; and
Xylα1,3Xylα1,3Glcβ-R;
wherein R is a linker.

3. The glycan array of claim 2, wherein said percentage of attached glycans comprising N-glycolylneuraminic acid (Neu5Gc) is from about 30% to about 50%.

4. The glycan array of claim 3, comprising at least one pair of attached glycans differing only by the substitution of a Neu5Gc residue for a Neu5Ac residue.

5. The glycan array of claim 4, comprising at least 40 pairs of attached glycans, wherein the members of each pair differ by the substitution of a Neu5Gc residue for a Neu5Ac residue.

6. The glycan array of claim 2, wherein said linker is selected from the group consisting of —O(CH₂)₂CH₂NH₂ and —O(CH₂)₃NHCOCH₂(OCH₂CH₂)₆NH₂.

7. A method of obtaining an anti-glycan antibody profile comprising:
a. obtaining a sample, wherein said sample comprises one or more antibodies,
b. contacting the glycan array of claim 1 with said sample,
c. obtaining glycan array binding results, and
d. preparing an anti-glycan antibody profile based on said glycan array binding results.

8. The method of claim 7, further comprising:
a. selecting at least one binding assay,
b. contacting said sample with said at least one binding assay,
c. obtaining results from said at least one binding assay, and
d. updating said anti-glycan antibody profile based on said results from said at least one binding assay.

9. The method of claim 8, wherein said at least one binding assay is selected from the group consisting of an alternative glycan array, an enzyme-linked immunosorbent assay (ELISA), a flow cytometry-based assay and a surface plasmon resonance (SPR)-based assay.

10. (canceled)

11. (canceled)

12. (canceled)

13. (canceled)

14. The method of claim 7, wherein said sample is obtained from an in vivo source and said in vivo source is selected from the group consisting of a human subject and a non-human animal subject.

15. (canceled)

16. The method of claim 14, wherein said sample is obtained from a human subject and wherein said sample is selected from the group consisting of blood, plasma, serum, cells, tissues, organs, mucus, cerebrospinal fluid, saliva and urine.

17. A method of diagnosing a disease, disorder and/or condition comprising the use of an anti-glycan antibody profile obtained according to claim 7.

18. The method of claim 17, wherein said disease, disorder and/or condition is selected from the group consisting of a cancer or cancer-related indication; an immune-related indication; a viral indication; a cardiovascular indication; and a gastrointestinal indication.

19. The method of claim 18, wherein said disease, disorder and/or condition comprises a cancer or cancer-related indication and wherein said anti-glycan antibody profile comprises an anti-tumor associated carbohydrate antigen (TACA) antibody profile.

20. A diagnostic kit comprising the glycan array of claim 1 and instructions for use thereof.

21. A method of preparing a diagnostic array comprising:
a. obtaining a glycan profile of a cancerous tissue;
b. selecting at least one glycan based on said glycan profile;
c. preparing a pH-optimized printing buffer, wherein the pH of said pH-optimized printing buffer stabilizes at least one chemical group on said at least one glycan; and
d. preparing a diagnostic array with said at least one glycan and said pH-optimized printing buffer.

22. The method of claim 21, wherein said at least one chemical group comprises a 9-O acetyl group.

23. A method of preparing a diagnostic array comprising:
a. obtaining a glycan profile of a cancerous tissue, wherein the glycan density of the cancerous tissue glycans is determined;
b. selecting at least one cancerous tissue glycan based on said glycan profile;
c. preparing a glycan density-optimized printing buffer; and
d. preparing a diagnostic array with said glycan density-optimized printing buffer.

24. The method of claim 23, wherein said cancerous tissue glycan comprises STn.

25. A diagnostic array prepared according to the method of claim 21.

26. A method of diagnosing cancer in a subject comprising:
a. obtaining a subject sample;
b. applying said subject sample to the diagnostic array of claim 25; and
c. detecting at least one anti-glycan antibody using said diagnostic array, thereby diagnosing cancer.

27. The method of claim 26, wherein said at least one anti-glycan antibody comprises an anti-STn antibody.
